SEQUENCE LISTING (82 sequences)

SEQ ID NO: 1 - protein, 10 aa, synthetic construct
  source 1..10: note = Antibody mAb-C VH CDR1 (Exemplary, AbM)
GFTFSWYYIH

SEQ ID NO: 2 - protein, 17 aa, synthetic construct
  source 1..17: note = Antibody mAb-C VH CDR2 (Exemplary, Kabat)
YIDSSSGYTEYADSVKG

SEQ ID NO: 3 - protein, 8 aa, synthetic construct
  source 1..8: note = Antibody mAb-C VH CDR3 (Exemplary, Kabat, AbM)
GGYLGMDY

SEQ ID NO: 4 - protein, 11 aa, synthetic construct
  source 1..11: note = Antibody mAb-C, mAb-P VL CDR1 (Exemplary, Kabat, AbM)
RASQSVSSAVA

SEQ ID NO: 5 - protein, 7 aa, synthetic construct
  source 1..7: note = Antibody mAb-C VL CDR2 (Exemplary, Kabat, AbM)
SASSLYS

SEQ ID NO: 6 - protein, 9 aa, synthetic construct
  source 1..9: note = Antibody mAb-C VL CDR3 (Exemplary, IMGT, Kabat, AbM)
QQSRSRPRT

SEQ ID NO: 7 - protein, 8 aa, synthetic construct
  source 1..8: note = Antibody mAb-C VH CDR1 (IMGT)
GFTFSWYY

SEQ ID NO: 8 - protein, 8 aa, synthetic construct
  source 1..8: note = Antibody mAb-C VH CDR2 (IMGT)
IDSSSGYT

SEQ ID NO: 9 - protein, 10 aa, synthetic construct
  source 1..10: note = Antibody mAb-C VH CDR3 (IMGT)
ARGGYLGMDY

SEQ ID NO: 10 - protein, 6 aa, synthetic construct
  source 1..6: note = Antibody mAb-C, mAb-P VL CDR1 (IMGT)
QSVSSA

SEQ ID NO: 11 - skipped

SEQ ID NO: 12 - protein, 5 aa, synthetic construct
  source 1..5: note = Antibody mAb-C VH CDR1 (Kabat)
WYYIH

SEQ ID NO: 13 - protein, 7 aa, synthetic construct
  source 1..7: note = Antibody mAb-C VH CDR1 (Chothia)
GFTFSWY

SEQ ID NO: 14 - protein, 4 aa, synthetic construct
  source 1..4: note = Antibody mAb-C VH CDR2 (Chothia)
SSSG

SEQ ID NO: 15 - protein, 6 aa, synthetic construct
  source 1..6: note = Antibody mAb-C VH CDR3 (Chothia)
GYLGMD

SEQ ID NO: 16 - protein, 7 aa, synthetic construct
  source 1..7: note = Antibody mAb-C, mAb-P VL CDR1 (Chothia)
SQSVSSA

SEQ ID NO: 17 - protein, 6 aa, synthetic construct
  source 1..6: note = Antibody mAb-C VL CDR3 (Chothia)
SRSRPR

SEQ ID NO: 18 - protein, 6 aa, synthetic construct
  source 1..6: note = Antibody mAb-C VH CDR1 (Contact)
SWYYIH

SEQ ID NO: 19 - protein, 13 aa, synthetic construct
  source 1..13: note = Antibody mAb-C VH CDR2 (Contact)
WVAYIDSSSGYTE

SEQ ID NO: 20 - protein, 9 aa, synthetic construct
  source 1..9: note = Antibody mAb-C VH CDR3 (Contact)
ARGGYLGMD

SEQ ID NO: 21 - protein, 7 aa, synthetic construct
  source 1..7: note = Antibody mAb-C, mAb-P VL CDR1 (Contact)
SSAVAWY

SEQ ID NO: 22 - protein, 10 aa, synthetic construct
  source 1..10: note = Antibody mAb-C VL CDR2 (Contact)
LLIYSASSLY

SEQ ID NO: 23 - protein, 8 aa, synthetic construct
  source 1..8: note = Antibody mAb-C VL CDR3 (Contact)
QQSRSRPR

SEQ ID NO: 24 - protein, 10 aa, synthetic construct
  source 1..10: note = Antibody mAb-C VH CDR2 (AbM)
YIDSSSGYTE

SEQ ID NO: 25 - protein, 117 aa, synthetic construct
  source 1..117: note = Antibody mAb-C VH Sequence
EVQLVESGGGLVQPGGSLRLSCAASGFTFSWYYIHWVRQAPGKGLEWVAYIDSSSGYTEYADSVKGRFTISADTSKNTAYLQMNSLRAEDTAVYYCARGGYLGMDYWGQGTLVTVSS

SEQ ID NO: 26 - protein, 107 aa, synthetic construct
  source 1..107: note = Antibody mAb-C VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQSRSRPRTFGQGTKVEIK

SEQ ID NO: 27 - protein, 10 aa, synthetic construct
  source 1..10: note = Antibody mAb-P VH CDR1 (Exemplary, AbM)
GFTFRSYYIH

SEQ ID NO: 28 - protein, 17 aa, synthetic construct
  source 1..17: note = Antibody mAb-P VH CDR2 (Exemplary, Kabat)
TISSYGVFTYYADSVKG

SEQ ID NO: 29 - protein, 10 aa, synthetic construct
  source 1..10: note = Antibody mAb-P VH CDR3 (Exemplary, Kabat, AbM)
GTTLTPVLDY

SEQ ID NO: 30 - protein, 7 aa, synthetic construct
  source 1..7: note = Antibody mAb-P VL CDR2 (Exemplary, Kabat, AbM)
SASSLSS

SEQ ID NO: 31 - protein, 9 aa, synthetic construct
  source 1..9: note = Antibody mAb-P VL CDR3 (Exemplary, IMGT, Kabat, AbM)
QQFGAEPIT

SEQ ID NO: 32 - protein, 8 aa, synthetic construct
  source 1..8: note = Antibody mAb-P VH CDR1 (IMGT)
GFTFRSYY

SEQ ID NO: 33 - protein, 8 aa, synthetic construct
  source 1..8: note = Antibody mAb-P VH CDR2 (IMGT)
ISSYGVFT

SEQ ID NO: 34 - protein, 12 aa, synthetic construct
  source 1..12: note = Antibody mAb-P VH CDR3 (IMGT)
ARGTTLTPVLDY

SEQ ID NO: 35 - protein, 5 aa, synthetic construct
  source 1..5: note = Antibody mAb-P VH CDR1 (Kabat)
SYYIH

SEQ ID NO: 36 - protein, 7 aa, synthetic construct
  source 1..7: note = Antibody mAb-P VH CDR1 (Chothia)
GFTFRSY

SEQ ID NO: 37 - protein, 4 aa, synthetic construct
  source 1..4: note = Antibody mAb-P VH CDR2 (Chothia)
SYGV

SEQ ID NO: 38 - protein, 8 aa, synthetic construct
  source 1..8: note = Antibody mAb-P VH CDR3 (Chothia)
TTLTPVLD

SEQ ID NO: 39 - protein, 6 aa, synthetic construct
  source 1..6: note = Antibody mAb-P VL CDR3 (Chothia)
FGAEPI

SEQ ID NO: 40 - protein, 6 aa, synthetic construct
  source 1..6: note = Antibody mAb-P VH CDR1 (Contact)
RSYYIH

SEQ ID NO: 41 - protein, 13 aa, synthetic construct
  source 1..13: note = Antibody mAb-P VH CDR2 (Contact)
WVATISSYGVFTY

SEQ ID NO: 42 - protein, 11 aa, synthetic construct
  source 1..11: note = Antibody mAb-P VH CDR3 (Contact)
ARGTTLTPVLD

SEQ ID NO: 43 - protein, 10 aa, synthetic construct
  source 1..10: note = Antibody mAb-P VL CDR2 (Contact)
LLIYSASSLS

SEQ ID NO: 44 - protein, 8 aa, synthetic construct
  source 1..8: note = Antibody mAb-P VL CDR3 (Contact)
QQFGAEPI

SEQ ID NO: 45 - protein, 10 aa, synthetic construct
  source 1..10: note = Antibody mAb-P VH CDR2 (AbM)
TISSYGVFTY

SEQ ID NO: 46 - protein, 119 aa, synthetic construct
  source 1..119: note = Antibody mAb-P VH Sequence
EVQLVESGGGLVQPGGSLRLSCAASGFTFRSYYIHWVRQAPGKGLEWVATISSYGVFTYYADSVKGRFTISADTSKNTAYLQMNSLRAEDTAVYYCARGTTLTPVLDYWGQGTLVTVSS

SEQ ID NO: 47 - protein, 107 aa, synthetic construct
  source 1..107: note = Antibody mAb-P VL Sequence
DIQMTQSPSSLSASVGDRVTITCRASQSVSSAVAWYQQKPGKAPKLLIYSASSLSSGVPSRFSGSRSGTDFTLTISSLQPEDFATYYCQQFGAEPITFGQGTKVEIK

SEQ ID NO: 48 - protein, 441 aa, synthetic construct
  source 1..441: note = mAb-C First polypeptide chain
DIQMTQSPSSLSASVGDRVTITCRASQSVSSAVAWYQQKPGKAPKLLIYSASSLYSGVPSRFSGSRSGTDFTLTISSLQPEDFATYYCQQSRSRPRTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 224 aa, synthetic construct
  source 1..224: note = mAb-C Second polypeptide chain
EVQLVESGGGLVQPGGSLRLSCAASGFTFSWYYIHWVRQAPGKGLEWVAYIDSSSGYTEYADSVKGRFTISADTSKNTAYLQMNSLRAEDTAVYYCARGGYLGMDYWGQGTLVTVSSAQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 441 aa, synthetic construct
  source 1..441: note = mAb-P Third polypeptide chain
DIQMTQSPSSLSASVGDRVTITCRASQSVSSAVAWYQQKPGKAPKLLIYSASSLSSGVPSRFSGSRSGTDFTLTISSLQPEDFATYYCQQFGAEPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 222 aa, synthetic construct
  source 1..222: note = mAb-P Fourth polypeptide chain
EVQLVESGGGLVQPGGSLRLSCAASGFTFRSYYIHWVRQAPGKGLEWVATISSYGVFTYYADSVKGRFTISADTSKNTAYLQMNSLRAEDTAVYYCARGTTLTPVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 52 - protein, 441 aa, synthetic construct
  source 1..441: note = mAb-C First polypeptide chain (silent Fc)
DIQMTQSPSSLSASVGDRVTITCRASQSVSSAVAWYQQKPGKAPKLLIYSASSLYSGVPSRFSGSRSGTDFTLTISSLQPEDFATYYCQQSRSRPRTFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 441 aa, synthetic construct
  source 1..441: note = mAb-P Third polypeptide chain (silent Fc)
DIQMTQSPSSLSASVGDRVTITCRASQSVSSAVAWYQQKPGKAPKLLIYSASSLSSGVPSRFSGSRSGTDFTLTISSLQPEDFATYYCQQFGAEPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 447 aa, synthetic construct
  source 1..447: note = mAb-C mAb Heavy Chain
EVQLVESGGGLVQPGGSLRLSCAASGFTFSWYYIHWVRQAPGKGLEWVAYIDSSSGYTEYADSVKGRFTISADTSKNTAYLQMNSLRAEDTAVYYCARGGYLGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, synthetic construct
  source 1..214: note = mAb-C mAb Light Chain
DIQMTQSPSSLSASVGDRVTITCRASQSVSSAVAWYQQKPGKAPKLLIYSASSLYSGVPSRFSGSRSGTDFTLTISSLQPEDFATYYCQQSRSRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 449 aa, synthetic construct
  source 1..449: note = mAb-P Heavy Chain
EVQLVESGGGLVQPGGSLRLSCAASGFTFRSYYIHWVRQAPGKGLEWVATISSYGVFTYYADSVKGRFTISADTSKNTAYLQMNSLRAEDTAVYYCARGTTLTPVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, synthetic construct
  source 1..214: note = mAb-P Light Chain
DIQMTQSPSSLSASVGDRVTITCRASQSVSSAVAWYQQKPGKAPKLLIYSASSLSSGVPSRFSGSRSGTDFTLTISSLQPEDFATYYCQQFGAEPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 323 aa, Homo sapiens
  source 1..323: note = Full-length amino acid sequence of human CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 59 - protein, 504 aa, Homo sapiens
  source 1..504: note = Full-length amino acid sequence of human SIRPalpha
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 60 - protein, 290 aa, Homo sapiens
  source 1..290: note = Full-length amino acid sequence of human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 61 - protein, 288 aa, Homo sapiens
  source 1..288: note = Full-length amino acid sequence of human PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 62 - protein, 222 aa, synthetic construct
  source 1..222: note = Exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 222 aa, synthetic construct
  source 1..222: note = Exemplary variant Fc region (silent Fc) sequence
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 110 aa, synthetic construct
  source 1..110: note = Exemplary variant CH2 domain (e.g., silent CH2) sequence
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAK

SEQ ID NO: 65 - protein, 330 aa, synthetic construct [sequence omitted]

SEQ ID NO: 66 - protein, 330 aa, synthetic construct
  source 1..330: note = Exemplary IgG heavy chain comprises any VH domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 107 aa, synthetic construct
  source 1..107: note = Exemplary light chain (e.g., for pairing with an IgG heavy chain) comprises any VL domain
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 226 aa, synthetic construct
  source 1..226: note = Phage display heavy chain
  VARIANT 31..35: note = X can be any amino acid
  VARIANT 50..66: note = X can be any amino acid
  VARIANT 99..112: note = X can be any amino acid
EVQLVESGGGLVQPGGSLRLSCAASGFTFSXXXXXWVRQAPGKGLEWVAXXXXXXXXXXXXXXXXXRFTISADTSKNTAYLQMNSLRAEDTAVYYCARXXXXXXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - protein, 214 aa, synthetic construct
  source 1..214: note = Phage display light chain
  VARIANT 89..97: note = X can be any amino acid
DIQMTQSPSSLSASVGDRVTITCRASQSVSSAVAWYQQKPGKAPKLLIYSASSLYSGVPSRFSGSRSGTDFTLTISSLQPEDFATYYCXXXXXXXXXFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 107 aa, synthetic construct
  source 1..107: note = Exemplary CH3 sequence
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 107 aa, synthetic construct
  source 1..107: note = Exemplary CH3 sequence
GQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 107 aa, synthetic construct
  source 1..107: note = Exemplary CH3 sequence
AQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 107 aa, synthetic construct
  source 1..107: note = Exemplary CH3 sequence
GQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 107 aa, synthetic construct
  source 1..107: note = Exemplary CH3 sequence
AQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 107 aa, synthetic construct
  source 1..107: note = Exemplary CH3 sequence
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 107 aa, synthetic construct
  source 1..107: note = Exemplary CH3 sequence
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 110 aa, synthetic construct; identical to SEQ ID NO: 64
  source 1..110: note = Exemplary CH2 sequence

SEQ ID NO: 78 - protein, 110 aa, synthetic construct
  source 1..110: note = Exemplary CH2 sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 79 - protein, 107 aa, synthetic construct [sequence omitted]

SEQ ID NO: 80 - protein, 103 aa, synthetic construct
  source 1..103: note = Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 81 - protein, 103 aa, synthetic construct
  source 1..103: note = Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 82 - protein, 103 aa, synthetic construct
  source 1..103: note = Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDRKVEPKSC

(19) United States
(12) Patent Application Publication
(10) Pub. No.: US 2025/0197502 A1
(43) Pub. Date: Jun. 19, 2025

(54) MULTISPECIFIC BINDING AGENTS AND USES THEREOF

(71) Applicant: EXELIXIS, INC., Alameda, CA (US)

(72) Inventors: Bryan GLASER, Madison, WI (US); Bonnie HAMMER, Madison, WI (US); Seema KANTAK, Pacifica, CA (US); Bee-Cheng SIM, Mountain View, CA (US); Justin A. WETTER, Madison, WI (US)

(73) Assignee: EXELIXIS, INC., Alameda, CA (US)

(21) Appl. No.: 18/836,677

(22) PCT Filed: Feb. 8, 2023
(86) PCT No.: PCT/US2023/062185

§ 371 Date: Aug. 7, 2024

Related U.S. Application Data
(60) Provisional application No. 63/425,960, filed on Nov. 16, 2022.
(60) Provisional application No. 63/308,485, filed on Feb. 9, 2022.

(51) Int. Cl. C07K 16/28; A61K 39/00; A61K 47/68; A61P 35/00

(52) U.S. Cl. CPC C07K 16/2827; A61K 47/6849; A61K 47/6879; A61P 35/00; C07K 16/2803; A61K 2039/505

(57) ABSTRACT

The present disclosure provides multispecific binding agents (e.g., antibodies, such as bispecific antibodies) that have a first binding domain that binds to CD47, including human CD47, and one or more additional binding domains that bind to one or more targets that are not CD47, such as PD-L1 and uses thereof.

### CROSS-REFERENCE TO RELATED APPLICATIONS

This application claims the benefit of U.S. Provisional Patent Application No. 63/425,960, filed Nov. 16, 2022 and U.S. Provisional Patent Application No. 63/308,485, filed Feb. 9, 2022, the disclosure of each of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14529-129-228_SEQ_LISTING.xml”, was created on Feb. 8, 2023 and is 96,794 bytes in size.

### FIELD

The present disclosure relates generally to multispecific binding agents, such as bispecific antibodies, that have a first binding domain that binds to CD47, including human CD47, and one or more additional binding domains that bind to one or more targets that are not CD47, such as PD-L1, and methods of their use.

### BACKGROUND

Tumor cells have been shown to utilize both innate and adaptive checkpoints to evade anti-tumor immune responses. CD47 and PD-L1 are two targets widely expressed on the cell surface of tumor cells, which may coordinately suppress innate and adaptive sensing, respectively, to evade immune control.

CD47 is a cell surface glycoprotein that functions as a regulator of phagocytosis mediated by cells of the innate immune system. CD47 interacts with multiple ligands, such as integrins, signal regulatory protein alpha (SIRPα), signal regulatory protein gamma (SIRPγ) and thrombospondins. CD47 inhibits phagocytosis by interacting with SIRPα on the surface of macrophages and dendritic cells, triggering a “don't eat me” signal.

Expressing CD47 enables tumor cells to evade phagocytosis and escape from innate immune surveillance. Thus, CD47 has been a target for possible therapeutics. However, CD47 is broadly expressed on normal cells, such as hematopoietic cells, red blood cells (RBCs) and platelets. The broad expression of CD47 by healthy cells presents safety and efficacy challenges because targeting CD47 with a neutralizing antibody could affect healthy cells, possibly leading to toxic effects. Additionally, broad expression of CD47 could also lead to a rapid elimination of CD47 binding agents, leading to poor pharmacokinetics and decreased efficacy.

Additionally, activation or loss of CD47 can result in enhanced proliferation in a cell type dependent mannor. For example, astrocytoma cells have been shown to have increased proliferation following activation of CD47 and TSP-1, whereas the normal astroglial cells have not. It has also been proposed that CD47 may facilitate proliferation of cancer cells through a PI3K/Akt pathway.

Many of the anti-CD47 antibodies that have been reported are known to cause agglutination of RBCs upon inhibiting CD47 binding to SIRPα, which significantly lowers the therapeutic effect of such antibodies.

Programmed death ligand 1 (PD-L1) is a cell surface glycoprotein ligand that specifically binds to programmed death receptor 1 (PD-1), a key immune checkpoint receptor. PD-1 is upregulated on activated T cells, B cells, and monocytes and mediates immunosuppression. While PD-L2, the other PD-1 ligand, is expressed primarily on activated antigen-presenting cells (APCs), PD-L1 is broadly expressed, including in cells of hematopoietic lineage, such as activated T cells, B cells, monocytes, dendritic cells and macrophages, and peripheral tissues such as heart, skeletal, muscle, placenta, lung, kidney and liver tissues.

The binding of PD-L1 to PD-1 is a negative checkpoint that can activate the downstream signaling of PD-1 receptor in T cells, thus inhibiting the proliferation, cytokine generation and release, and cytotoxicity of T cells. This inhibition of T cell activation and secretion of effector cytokines can prevent autoimmunity and chronic infection.

However, many tumor cells use this mechanism to protect themselves from immune attack, resulting in tumor immune evasion. Many cancers overexpress PD-L1, and its overexpression is often associated with poor prognosis. In cancer, the PD-1/PD-L1 interaction stimulates the downstream signals to suppress T cell activation, resulting in tumor cell survival.

The blockade of PD-1 interaction with its ligands has been proposed as an immunotherapeutic method of enhancing T cell immune responses against tumor cells. Current strategies of PD-1/PD-L1 based immunotherapy have shown efficacy in treating some advanced carcinoma, but have limited effects on many solid tumors and on certain PD-L1 functions. Accordingly, there remains an urgent need in the art for agents that can inhibit or prevent PD-1/PD-L1 interaction.

Accordingly, there remains a need in the art for agents that overcome these concerns and which target CD47 and other targets, such as PD-L1, to treat, prevent, or alleviate immune cell dysfunctional diseases, disorders, or conditions, including those involving tumor cells expressing CD47 and/or PD-L1. The multispecific binding agents, compositions and methods provide herein satisfy this need and provide related advantages.

### SUMMARY

The present disclosure provides multispecific binding agents (e.g., antibodies, such as bispecific antibodies) that have a first binding domain that binds to CD47, including human CD47, and one or more additional binding domains that bind to one or more targets that are not CD47 (e.g., PD-L1). Such agents include multispecific antibodies (e.g., antibodies, such as bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1). Such agents, in some embodiments, include multispecific antibodies (e.g., bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1), wherein the first binding domain comprises: a heavy chain variable (VH) region comprising a VH CDR1, a VH CDR2, and a VH CDR3 amino acid sequence set forth in Table 1; and a light chain variable (VL) region comprising a VL CDR1, a VL CDR2, and a VL CDR3 amino acid sequence set forth in Table 1, or wherein the multispecific antibodies compete for the binding of human CD47 with an antibody having a heavy chain variable region and a light chain variable region described herein (e.g., Table 1).

The present disclosure also provides nucleic acids encoding a multispecific binding agent provided herein (e.g., an antibody or fragment thereof), vectors comprising one or more of such nucleic acids, and cells expressing the same.

The present disclosure also provides compositions comprising a multispecific binding agent described herein. Such compositions, in some embodiments, include multispecific antibodies (e.g., antibodies, such as bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1). Such compositions, in some embodiments, include multispecific antibodies (e.g., antibodies, such as bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1), wherein the first binding domain comprises: a heavy chain variable (VH) region comprising a VH CDR1, a VH CDR2, and a VH CDR3 amino acid sequence set forth in Table 1; and a light chain variable (VL) region comprising a VL CDR1, a VL CDR2, and a VL CDR3 amino acid sequence set forth in Table 1, or wherein the multispecific antibodies compete for the binding of human CD47 with an antibody having a heavy chain variable region and a light chain variable region described herein (e.g., Table 1).

The present disclosure also provides methods of treating, preventing, or alleviating an immune cell dysfunctional disease, disorder or condition (e.g., a phagocytic cell dysfunctional disease, disorder, or condition or a T cell dysfunctional disease, disorder, or condition), including one or more symptoms of the immune cell dysfunctional disease, disorder, or condition with a multispecific binding agent or a composition comprising the multispecific binding agent, including a bispecific antibody or composition comprising the bispecific antibody, as described herein. Such compositions include multispecific antibodies (e.g., antibodies, such as bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1). Such compositions, in some embodiments, include multispecific antibodies (e.g., antibodies, such as bispecific antibodies) that bind to CD47 and one or more additional targets that are not CD47 (e.g., PD-L1), for example, multispecific antibodies that have a first binding domain that binds to CD47, including human CD47, and one or more additional binding domains that bind to one or more targets that are not CD47 (e.g., PD-L1) and compete for the binding of human CD47 with an antibody having a heavy chain variable region and a light chain variable region described herein (e.g., Table 1).

### BRIEF DESCRIPTION OF THE DRAWINGS

FIGS. 1A-1E illustrate exemplary results from cell binding assays, further described in Example 2. FIG. 1A shows bsAb1's binding to PD-L1-expressing MDA-MB-231 cells (referred to herein as PDL1-MDA-MB-231 cells). FIG. 1B shows bsAb1's binding to NCI-H292. FIG. 1C shows bsAb1's binding to HT-1080. FIG. 1D shows bsAb1's lack of binding to RBCs. FIG. 1E shows bsAb1's binding to NCI-H292 with or without RBCs.

FIGS. 2A and 2B illustrate two exemplary results from CD47/SIRPα inhibiting assays, further described in Example 3.

FIGS. 3A and 3B illustrate two exemplary results from PD-L1/PD-1 inhibiting assays, further described in Example 3.

FIGS. 4A-4D illustrate exemplary results from phagocytosis assays, further described in Example 4.

FIGS. 5A-5D illustrate exemplary results from red blood cell agglutination assays, further described in Example 5.

FIG. 6 illustrates exemplary results from SEC chromatography, further described in Example 6.

FIG. 7 illustrates exemplary results from HIC chromatography, further described in Example 6.

FIG. 8 illustrates exemplary results from SMAC chromatography, further described in Example 6.

FIGS. 9A-9B illustrate exemplary results from in vivo efficacy study, further described in Example 7.

FIGS. 10A-10C illustrate exemplary results tested in non-human primates (NHP), further described in Example 8.

FIG. 11 illustrates an exemplary Biacore sensorgram of bsAb1 binding to human PD-L1, further described in Example 1.

### DETAILED DESCRIPTION

The present disclosure provides multispecific binding agents (e.g., antibodies, such as bispecific antibodies) that bind to CD47 and one or more additional targets that are not CD47 (e.g., PD-L1). Such multispecific binding agents include antibodies (e.g., antibodies, such as bispecific antibodies) that bind to CD47, including antibodies that bind to human CD47, and one or more additional targets that are not CD47 (e.g., PD-L1). Such multispecific binding agents are useful in compositions and in methods of treating, preventing, or alleviating an immune cell dysfunctional disease, disorder or condition (e.g., a phagocytic cell dysfunctional disease, disorder, or condition or a T cell dysfunctional disease, disorder, or condition), including one or more symptoms of the disease, disorder, or condition. Phagocytic cell dysfunctional diseases, disorders, and conditions include tumor immunity and associated cancers, including, but not limited to, any cancer wherein the tumor cells express or overexpress CD47. T cell dysfunctional diseases, disorders, and conditions include tumor immunity and associated cancers, including, but not limited to, any cancer wherein the tumor cells express or overexpress PD-L1. Such CD47 and/or PD-L1 expressing tumor cells may help tumor cells escape immune surveillance and clearance (e.g., tumor immunity). In addition, multispecific binding agents described herein, such as multispecific antibodies (e.g., antibodies, such as bispecific antibodies), that bind to CD47 and one or more additional targets that are not CD47 (e.g., PD-L1), are useful to inhibit SIRPα signaling and/or PD-1 signaling, enhance phagocytic cell function and/or immune surveillance, and enhance removal of tumor cells. Multispecific binding agents (e.g., antibodies, such as bispecific antibodies) described herein, such as multispecific binding antibodies (e.g., bispecific antibodies), are useful in compositions and in methods for enhancing phagocytic cell function and T cell function, including the upregulation of cell-mediated immune responses.

Provided herein among others are binding agents comprising (i) a first means for inhibiting the interaction between PD-L1 and PD-1, and (ii) a second means for inhibiting the interaction between CD47 and SIRPα. In some embodiments, the first means has a high affinity to PD-L1 (such as a human PD-L1 or a cyno PD-L1) and the second means has a detuned and/or moderate affinity to CD47 (such as a human CD47 or a cyno CD47). In some embodiments, the second means enables tight binding to tumor cells while minimizing binding to red blood cells (RBCs). In some embodiments, the first means has a KD for its interaction with PD-L1 of lower than 1 nM, or lower than 0.1 nM, such as about 0.05 nM; and the second means has a KD for its interaction with CD47 of (i) higher than 0.1 nM, or higher than 1 nM, or higher than 5 nM, or higher than 10 nM, or higher than 15 nM, or higher than 20 nM, and (ii) lower than 1 μM, or lower than 500 nM, or lower than 100 nM, or lower than 50 nM, or lower than 30 nM. In some embodiments, the second means has a KD for its interaction with CD47 of about 0.1 nM to about 1 μM, including each number and range therebetween, such as about 1 nM to about 100 nM, or about 10 nM to about 50 nM, or about 20 to about 30 nM. In some embodiments, the binding agents are bispecific antibodies comprising a binding domain that binds to PD-L1 and a binding domain that binds to CD47. Nucleic acid encoding the present binding agents, pharmaceutical compositions comprising the present binding agents or nucleic acids, and uses of the binding agents and pharmaceutical compositions are also provided herein.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “CD47,” “Cluster of Differentiation 47,” or “CD47 polypeptide” and similar terms refers to a polypeptide (“polypeptide” and “protein” are used interchangeably herein) or any native CD47 from any vertebrate source, including mammals such as primates (e.g., humans, cynomolgus monkey (cyno)), dogs, and rodents (e.g., mice and rats), unless otherwise indicated. CD47, also known in the art as integrin associated protein (IAP), has an extracellular N-terminal IgV domain, five transmembrane domains, and a short C-terminal intracellular tail. The term CD47 encompasses “full-length,” unprocessed CD47, as well as any form of CD47 or any fragment thereof that results from processing in the cell, including the four known alternatively spliced isoforms of CD47 that differ in the length of the intracellular tail. The term CD47 also encompasses naturally occurring variants of CD47, such as SNP variants, splice variants and allelic variants. CD47 is known in the art to interact with SIRPα and this interaction leads to cell signaling that includes, among other things, inhibition of phagocytosis by macrophages. The full-length amino acid sequence of human CD47 is provided below (exemplary extracellular domain=underline text):

(SEQ ID NO: 58)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFVASNQKTIQPPRKAVEEPLNAFKE
SKGMMNDE

Other related CD47 polypeptides that are also encompassed by the term CD47 include fragments, derivatives (e.g., substitution, deletion, truncations, and insertion variants), fusion polypeptides, and interspecies homologs that retain CD47 activity and/or are sufficient to generate an anti-CD47 immune response. As those skilled in the art will appreciate, a multispecific binding agent (e.g., an antibody, such as a bispecific antibody) described herein can bind to a CD47 polypeptide, a CD47 polypeptide fragment, a CD47 antigen, and/or a CD47 epitope. An epitope may be part of a larger CD47 antigen, which may be part of a larger CD47 polypeptide fragment, which, in turn, may be part of a larger CD47 polypeptide. CD47 may exist in a native or denatured form. CD47 polypeptides described herein may be isolated from a variety of sources, such as from human tissue types or from another source, or prepared by recombinant or synthetic methods. A CD47 polypeptide may comprise a polypeptide having the same amino acid sequence as a corresponding CD47 polypeptide derived from nature. Orthologs to the CD47 polypeptide are also well known in the art.

The term “SIRPα,” “Signal-regulatory protein alpha,” or “Signal-regulatory protein α” and similar terms refers to a polypeptide (“polypeptide” and “protein” are used interchangeably herein) or any native SIRPα from any vertebrate source, including mammals such as primates (e.g., humans, cynomolgus monkey (cyno)), dogs, and rodents (e.g., mice and rats), unless otherwise indicated. SIRPα has an extracellular region, which includes three immunoglobulin superfamily domains-single V-set and two C1-set IgSF domains, a transmembrane domain and a cytoplasmic region containing an immunoreceptor tyrosine-based inhibition motif (ITIM). The term SIRPα also encompasses naturally occurring variants of SIRPα, such as SNP variants, splice variants and allelic variants. SIRPα is known in the art to interact with CD47, leading to phosphorization of the ITIM, which mediates its association with the phosphatase SH2-domain-containing protein tyrosine phosphatase 2 (SHP2). The full-length amino acid sequence of human SIRPα is provided below:

(SEQ ID NO: 59)
MEPAGPAPGRLGPLLCLLLAASCAWSGVAGEEELQVIQPDKSVLV
AAGETATLRCTATSLIPVGPIQWFRGAGPGRELIYNQKEGHFPRV
TTVSDLTKRNNMDFSIRIGNITPADAGTYYCVKFRKGSPDDVEFK
SGAGTELSVRAKPSAPVVSGPAARATPQHTVSFTCESHGFSPRDI
TLKWFKNGNELSDFQTNVDPVGESVSYSIHSTAKVVLTREDVHSQ
VICEVAHVTLQGDPLRGTANLSETIRVPPTLEVTQQPVRAENQVN
VTCQVRKFYPQRLQLTWLENGNVSRTETASTVTENKDGTYNWMSW
LLVNVSAHRDDVKLTCQVEHDGQPAVSKSHDLKVSAHPKEQGSNT
AAENTGSNERNIYIVVGVVCTLLVALLMAALYLVRIRQKKAQGST
SSTRLHEPEKNAREITQDTNDITYADLNLPKGKKPAPQAAEPNNH
TEYASIQTSPQPASEDTLTYADLDMVHLNRTPKQPAPKPEPSFSE
YASVQVPRK.

The term “Programmed Cell Death Ligand-1 (PD-L1),” “Programmed Death Ligand-1,” “PD-1 ligand 1” or similar terms refers to a polypeptide (“polypeptide” and “protein” are used interchangeably herein) or any native PD-L1 from any vertebrate source, including mammals such as primates (e.g., humans, cynomolgus monkey (cyno)), dogs, and rodents (e.g., mice and rats), unless otherwise indicated. PD-L1, also known as cluster of differentiation 274 (CD274) or B7 homolog 1 (B7-H1) is a protein that in humans is encoded by the CD274 gene. PD-L1 is one of two naturally-occurring cell surface glycoprotein ligands for PD-1 (the other is PD-L2). Like PD-1, PD-L1 belongs to the immunoglobulin superfamily and consists of two extracellular Ig domains, an N-terminal V domain, and a C-terminal constant domain. PD-L1 is known in the art to downregulate T cell activation and cytokine secretion upon binding to PD-1. The term PD-L1 encompasses “full-length” PD-L1, as well as any form of PD-L1 or any fragment thereof that results from processing in the cell. The term PD-L1 also encompasses naturally occurring variants of PD-L1, such as SNP variants, splice variants and allelic variants. The full-length amino acid sequence of human PD-L1 is provided below (exemplary extracellular domain=underline text):

(SEQ ID NO: 60)
MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTTNEIFYCTFRRLDPEENHTAELV
IPELPLAHPPNERTHLVILGAILLCLGVALTFIFRLRKGRMMDVK
KCGIQDTNSKKQSDTHLEET.

Other related PD-L1 polypeptides that are also encompassed by the term PD-L1 include fragments, derivatives (e.g., substitution, deletion, truncations, and insertion variants), fusion polypeptides, and interspecies homologs that retain PD-L1 activity and/or are sufficient to generate an anti-PD-L1 immune response. As those skilled in the art will appreciate, a PD-L1 binding agent (e.g., an antibody) described herein can bind to a PD-L1 polypeptide, a PD-L1 polypeptide fragment, a PD-L1 antigen, and/or a PD-L1 epitope. An epitope may be part of a larger PD-L1 antigen, which may be part of a larger PD-L1 polypeptide fragment, which, in turn, may be part of a larger PD-L1 polypeptide. PD-L1 may exist in a native or denatured form. PD-L1 polypeptides described herein may be isolated from a variety of sources, such as from human tissue types or from another source, or prepared by recombinant or synthetic methods. A PD-L1 polypeptide may comprise a polypeptide having the same amino acid sequence as a corresponding PD-L1 polypeptide derived from nature. Orthologs to the PD-L1 polypeptide are also well known in the art.

The term “Programmed Cell Death-1 (PD-1),” “Programmed Death-1,” “PD-1 receptor” or similar terms refers to a polypeptide (“polypeptide” and “protein” are used interchangeably herein) or any native PD-1 from any vertebrate source, including mammals such as primates (e.g., humans, cynomolgus monkey (cyno)), dogs, and rodents (e.g., mice and rats), unless otherwise indicated. PD-1, also known as CD279 (cluster of differentiation 279), is an immunoinhibitory receptor belonging to the CD28 family. PD-1 is expressed predominantly on previously activated T cells in vivo, and binds to two ligands, PD-L1 and PD-L2. PD-1 belongs to the immunoglobulin superfamily and consists of two extracellular Ig domains, an N-terminal V domain, and a C-terminal constant domain. PD-1 contains two cytoplasmic tyrosine-based signaling motifs, an immunoreceptor tyrosine-based inhibition motif (ITIM) and an immunoreceptor tyrosine-based switch motif (ITSM). The term PD-1 encompasses “full-length” PD-1, as well as any form of PD-1 or any fragment thereof that results from processing in the cell. The term PD-1 also encompasses naturally occurring variants of PD-1, such as SNP variants, splice variants and allelic variants. Following T cell stimulation, PD-1 is known in the art to recruit the tyrosine phosphatase SHP-2 to the ITSM motif within its cytoplasmic tail, leading to, among other things, the dephosphorylation of effector molecules such as CD3 Zeta, PKC theta and ZAP70 that are involved in the CD3 T cell signaling cascade (Carter et al. (2002) Eur J Immunol 32:634-43). The full-length amino acid sequence of human PD-1 is provided below:

(SEQ ID NO: 61)
MQIPQAPWPVVWAVLQLGWRPGWFLDSPDRPWNPPTFSPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL.

As used herein, the term “binding agent” or a grammatical equivalent thereof refers to a molecule (e.g., an antibody, such as a bispecific antibody) with one or more antigen binding sites that binds an antigen. In some embodiments, a multispecific binding agent as described herein is an antibody, antibody fragment, or other peptide-based molecule that binds to CD47 and/or PD-L1, such as human CD47 and/or PD-L1.

The term “antibody,” “immunoglobulin,” or “Ig” is used interchangeably herein, and is used in the broadest sense and specifically covers, for example, polyclonal antibodies, monoclonal antibodies (including agonist, antagonist, neutralizing antibodies, full-length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full-length heavy and/or light chains. The present disclosure also includes antibody fragments (and/or polypeptides that comprise antibody fragments) that retain CD47 and/or PD-L1 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) chain variable domains. For example, the present disclosure also includes tetrameric antibodies comprising two heavy chain and two light chain molecules, an antibody light chain monomer, and an antibody heavy chain monomer. Thus, for example, the V region domain may be dimeric and contain VH-VH, VH-VL, or VL-VL dimers that bind CD47 or PD-L1. If desired, the VH and VL chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23 (9): 1126-1136, 2005). The binding agent, in some embodiments, contains a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monospecific” when used in reference to a binding agent (e.g., an antibody) as used herein denotes a binding agent that has one or more binding sites each of which binds to the same epitope of the same antigen.

The term “multispecific” when used in reference to a binding agent (e.g., an antibody) means that the binding agent has binding specificities for at least two different antigens or at least two different epitopes on the same antigen (e.g., a bispecific antibody directed to CD47 with a first binding site for a first epitope of a CD47, and a second binding site for a second epitope of CD47).

The term “bispecific” when used in reference to a binding agent (e.g., an antibody) means that the binding agent is able to specifically bind to two distinct antigenic determinants (e.g., epitopes), for example, two binding sites each formed by a pair of an antibody heavy chain variable domain (VH) and an antibody light chain variable domain (VL) binding to different antigens or to different epitopes on the same antigen. Such a bispecific binding agent may have a 1+1 format. Other bispecific binding agent (e.g., an antibody) formats may be 2+1 or 1+2 formats (comprising two binding sites for a first antigen or epitope and one binding site for a second antigen or epitope) or 2+2 formats (comprising two binding sites for a first antigen or epitope and two binding sites for a second antigen or epitope). When a bispecific binding agent (e.g., an antibody) comprises two antigen binding sites, each may bind to a different antigenic determinant. Such a bispecific binding agent (e.g., an antibody) may bind to two different epitopes on the same antigen (e.g., epitopes on CD47).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component. Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains.

As used herein, an “antigen” is a moiety or molecule that contains an epitope to which a binding agent (e.g., an antibody, such as a bispecific antibody) can bind. As such, an antigen can be bound by an antibody. In some embodiments, the antigen, to which a binding agent (e.g., an antibody, such as a bispecific antibody) described herein binds, is CD47 (e.g., human CD47) and/or PD-L1 (e.g., human PD-L1), or a fragment of each thereof.

As used herein, an “epitope” (which is used interchangeably with “antigenic determinant”) is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human CD47 and/or PD-L1. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance, that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, BIACORE™, KinExA 3000 instrument (Sapidyne Instruments, Boise, ID), or other assays known in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other non-CD47 and/or non-PD-L1 proteins. In some embodiments “specifically binds” means, for instance, that a polypeptide or molecule binds a protein or target with a KD of about 0.1 mM or less, but more usually less than about 1 μM. In some embodiments, “specifically binds” means that a polypeptide or molecule binds a target with a KD of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by assays known in the art, for example by a binding assay. The K_{D} values reported herein were determined by biolayer interferometry (BLI) using, for example, the OctetQK384 system (ForteBio, Menlo Park, CA). Alternatively, the K_{D} may be also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by Biacore, using, for example, a BIACORE™-2000 or a BIACORE™-3000 BIAcore, Inc., Piscataway, NJ). An “on-rate” or “rate of association” or “association rate” or “Kon,” as well as an “off-rate” or “rate of dissociation” or “dissociation rate” or “Koff,” can also be determined with the same SPR or BLI techniques described above using, for example, the OctetQK384 system (ForteBio, Menlo Park, CA) or a BIACORE™-2000 or a BIACORE™-3000 (BIAcore, Inc., Piscataway, NJ), respectively.

The term “compete” when used in the context of multispecific binding agents (e.g., antibodies, such as bispecific antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen binding protein, such as a reference antibody) to a common antigen (e.g., CD47 or PD-L1). Numerous types of competitive binding assays can be used to determine if a test binding agent competes with a reference molecule for binding to CD47 (e.g., human CD47) or PD-L1 (e.g., human PD-L1). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 and Cheung, et al., (1990) Virology 176:546-552) solid phase direct labeled assay, solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., CD47, such as human CD47) bound to a solid surface or cells bearing either an unlabelled test antigen binding protein (e.g., test CD47 antibody) or a labeled reference antigen binding protein (e.g., reference CD47 antibody). Competitive inhibition may be measured by determining the amount of label bound to the solid surface or cells in the presence of the test antigen binding protein. Usually, the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instances, binding is inhibited by at least 80%, 85%, 90%, 95%, 96% or 97%, 98%, 99% or more.

As used herein, the term “constant region” or “constant domain” is a well-known antibody term of art and refers to an antibody portion, e.g., for example, a carboxyl terminal portion of a light and/or heavy chain which is not directly involved in binding of an antibody to antigen but which can exhibit various effector functions, such as interaction with the Fc receptor. The term includes the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and varied with the antibody isotype. Examples of antibody effector functions include: C1q binding and complement dependent cytotoxicity; Fc receptor binding; antibody-dependent cell-mediated cytotoxicity (ADCC); phagocytosis (such as antibody-dependent cellular phagocytosis (ADCP)); down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. An exemplary Fc region sequence is provided below (CH2 domain=bold text; CH3 domain=underline text):

(SEQ ID NO: 62)
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK.

A “functional Fc region” possesses an “effector function” of a native sequence Fc region. Exemplary “effector functions” include Clq binding; complement dependent cytotoxicity (CDC); Fc receptor binding; antibody-dependent cell-mediated cytotoxicity (ADCC); phagocytosis (such as ADCP); down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification (e.g., substituting, addition, or deletion), preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region herein can possess at least about 80% homology with a native sequence Fc region and/or with an Fc region of a parent polypeptide, or at least about 90% homology therewith, for example, at least about 95% homology therewith. The variant Fc region herein described herein may have a loss of effector function (e.g., silent Fc). An exemplary variant Fc region (“silent Fc”) sequence is provided below (CH2 domain=bold text with amino acid changes underlined; CH3 domain=underline text):

(SEQ ID NO: 63)
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALKAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK.

An exemplary variant CH2 domain (e.g., silent CH2) sequence useful for multispecific (e.g., bispecific) binding agents described herein is provided below (amino acid changes underlined):

(SEQ ID NO: 64)
APEAAGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALKAPIEKTISKAK.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) of lambda (λ) based on the amino acid sequence of the constant domains. Light chain amino acid sequences are well known in the art.

The terms “antigen binding fragment,” “antigen binding domain,” “antigen binding region,” and similar terms refer to that portion of an antibody, which comprises the amino acid residues that interact with an antigen and confer on the binding fragment, domain, or region its specificity and affinity for the antigen (e.g., the CDRs). “Antigen binding fragment” as used herein includes “antibody fragment,” which comprises a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen binding domains that bind to a CD47 antigen and one or more antigen binding domains that bind to one or more targets other than CD47 (e.g., PD-L1).

Antibodies can be of any type (e.g., IgG, IgE, IgM, IgD, IgA or IgY), any class, (e.g., IgG1, IgG2, IgG3, IgG4, IgA1 or lgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In some embodiments, an antibody is a 4-chain antibody unit comprising two heavy (H) chain/light (L) chain pairs, wherein the amino acid sequences of the H chains are identical and the amino acid sequences of the L chains are identical. In some embodiments, the H and L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprises a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to CD47, such as human CD47, meaning that the antibody or fragment thereof binds CD47 with greater affinity than it binds to an unrelated control protein and/or binds human CD47 with greater affinity than it binds to an unrelated control protein. For example, the antibody or fragment thereof may specifically recognize and bind CD47 or a portion thereof. “Specific binding” means that the antibody or fragment thereof binds to CD47 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind CD47 substantially exclusively (e.g., is able to distinguish CD47 from other known polypeptides, for example, by virtue of measurable differences in binding affinity). In some embodiments, a multispecific binding agent (e.g., an antibody, such as a bispecific antibody) may react with CD47 sequences other than human CD47 sequences (e.g., cynomolgous CD47 sequences).

Additionally or alternatively, an antibody or fragment thereof may preferentially bind to PD-L1, such as human PD-L1, meaning that the antibody or fragment thereof binds PD-L1 with greater affinity than it binds to an unrelated control protein and/or binds human PD-L1 with greater affinity than it binds to an unrelated control protein. For example, the antibody or fragment thereof may specifically recognize and bind PD-L1 or a portion thereof. “Specific binding” means that the antibody or fragment thereof binds to PD-L1 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind PD-L1 substantially exclusively (e.g., is able to distinguish PD-L1 from other known polypeptides, for example, by virtue of measurable differences in binding affinity). In some embodiments, a multispecific binding agent (e.g., an antibody, such as a bispecific antibody) may react with PD-L1 sequences other than human PD-L1 sequences (e.g., cynomolgous PD-L1 sequences).

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and is used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four FRs (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991). The constant regions are not involved directly in binding an antibody to an antigen, but exhibit various effector functions, such as participation of the antibody in antibody dependent cellular cytotoxicity (ADCC), ADCP and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions: three in the VH (H1, H2, H3), and three in the VL (L1, L2, L3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B: if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT) Information System® (Lefranc et al., Dev. Comp. Immunol. 27 (1): 55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309:657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. An exemplary system, shown herein, combines Kabat and Chothia.

| | Exemplary | IMGT | Kabat | AbM | Chothia | Contact |
|---|---|---|---|---|---|---|
| VH CDR1 | 26-35 | 27-38 | 31-35 | 26-35 | 26-32 | 30-35 |
| VH CDR2 | 50-65 | 56-65 | 50-65 | 50-58 | 52a/53-55 | 47-58 |
| VH CDR3 | 95-102 | 105-117 | 95-102 | 95-102 | 96-101 | 93-101 |
| VL CDR1 | 24-34 | 27-38 | 24-34 | 24-34 | 26-32 | 30-36 |
| VL CDR2 | 50-56 | 56-65 | 50-56 | 50-56 | 50-52 | 46-55 |
| VL CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “vector” refers to a substance that is used to carry or include a nucleic acid sequence, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well-known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a multispecific binding agent as described herein), and it is further understood that expression levels can be optimized to obtain sufficient expression using methods well known in the art.

The term “treating” or any grammatical variation thereof refers to reducing and/or ameliorating the severity and/or duration of a given disease, disorder or condition, and/or a symptom related thereto, such as (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of a multispecific binding agent described herein).

An “immune cell dysfunctional disease,” “immune cell dysfunctional disorder” and “immune cell dysfunctional condition” are used interchangeably and refer to any disease, disorder or condition that is completely or partially caused by or is the result of improper signaling to an immune cell and/or alternatively any disease, disorder, or condition in which it is desirable to inhibit the in vivo effects of the interaction of an immune cell receptor (e.g., SIRPα or PD-1) with its ligand (e.g., CD47 or PD-L1). An immune cell dysfunctional disease includes a phagocytic cell dysfunctional disease and a T cell dysfunctional disease.

A “phagocytic cell dysfunctional disease,” “phagocytic cell dysfunctional disorder” and “phagocytic cell dysfunctional condition” are used interchangeably and refer to any disease, disorder or condition that is completely or partially caused by or is the result of CD47 (e.g., abnormal expression of CD47) or the interaction of CD47 with SIRPα and/or alternatively any disease, disorder, or condition in which it is desirable to inhibit the in vivo effects of the interaction of CD47 with SIRPα. A phagocytic cell dysfunctional disease includes a disease, disorder or condition that is characterized by or associated with decreased phagocytic activity of immune cells (e.g., neutrophils, macrophages, dendritic cells, B lymphocytes). In some embodiments, a phagocytic cell dysfunctional disease is a disease, disorder or condition that is specifically associated with inappropriately increased signaling through SIRPα. In some embodiments, a phagocytic cell dysfunctional disease is one in which phagocytic cells (e.g., macrophages) have decreased ability to ingest or engulf other cells (e.g., a tumor cell) or particles. In some embodiments, the decreased ability to ingest or engulf other cells or particles results in ineffective control of a pathogen or tumor, including but not limited to tumors expressing CD47. Examples of a phagocytic cell dysfunctional disease characterized by phagocytic cell dysfunction include unresolved acute infection, chronic infection and tumor immunity (e.g., any cancers, including but not limited to cancers that express or overexpress CD47).

A “T cell dysfunctional disease,” “T cell dysfunctional disorder” and “T cell dysfunctional condition” are used interchangeably and refer to any disease, disorder or condition of T cells characterized by decreased responsiveness to antigenic stimulation. A T cell dysfunctional disease includes a disease, disorder or condition that is completely or partially caused by or is the result of PD-L1 (e.g., abnormal expression of PD-L1) or the interaction of PD-L1 with PD-1 and/or alternatively any disease, disorder, or condition in which it is desirable to inhibit the in vivo effects of the interaction of PD-L1 with PD-1. In some embodiments, a T cell dysfunctional disease is a disease, disorder or condition that is specifically associated with inappropriately increased signaling through PD-1. In some embodiments, a T cell dysfunctional disease is one in which T cells are anergic or have decreased ability to secrete cytokines, proliferate, or execute cytolytic activity. In some embodiments, the decreased responsiveness results in ineffective control of a pathogen or tumor, including but not limited to tumors expressing PD-L1. Examples of a T cell dysfunctional disease characterized by T cell dysfunction include unresolved acute infection, chronic infection and tumor immunity (e.g., from any cancers, including but not limited to cancers that express or overexpress PD-L1).

“Tumor immunity” refers to the process in which tumors evade immune recognition and clearance. Thus, as a therapeutic concept, tumor immunity is “treated” when such evasion is attenuated and the tumors are recognized and attacked by the immune system. Examples of tumor recognition include tumor binding, tumor shrinkage and tumor clearance.

“Enhancing T cell function” means to induce, cause or stimulate a T cell to have a sustained or increased biological function, or renew or reactivate exhausted or inactive T cells. Examples of enhancing T cell function include: increased secretion of cytokines (e.g., TNFα, IFNγ) from CD8⁺ T cells, increased proliferation, and increased antigen responsiveness (e.g., tumor cell removal) relative to such levels before the intervention. In some embodiments, the level of enhancement is as least 50%, alternatively 60%, 70%, 80%, 90%, 100%, 120%, 150%, or 200%. The manner of measuring this enhancement is known to one of ordinary skill in the art.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an antibody described herein). A “therapeutically effective amount” of a substance/molecule/agent of the present disclosure (e.g., a binding agent, such as a monoclonal antibody, a monospecific antibody, or a bispecific antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s). The full therapeutic or prophylactic effect does not necessarily occur by administration of one dose, and may occur only after administration of a series of doses. Thus, a therapeutically or prophylactically effective amount may be administered in one or more administrations.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsions, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of a multispecific binding agent (e.g., an antibody, such as a bispecific antibody), for example, in isolated or purified form, together with a suitable amount of carrier so as to provide the form for proper administration to the subject (e.g., patient). The formulation should suit the mode of administration.

The terms “about” and “approximately” mean within 20%, within 15%, within 10%, within 9%, within 8%, within 7%, within 6%, within 5%, within 4%, within 3%, within 2%, within 1%, or less variation of a given value or range.

As used herein, comparative terms as used herein, such as reduce, decrease, increase, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 fold, or about 3 fold, or about 4 fold, or about 5 fold, or about 10 fold, or about 20 fold, or about 30 fold, or about 40 fold, or about 100 fold or higher than the reference. In some embodiments, such variation can refer to about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 95%, or about 96%, or about 97%, or about 98%, or about 99% of the reference.

As used in the present disclosure and claims, the singular forms “a”, “an” and “the” include plural forms unless the context clearly dictates otherwise.

In some embodiments, the terms “first,” “second,” “third,” “fourth” and similar in a component name are used to distinguish and identify more than one component sharing certain identity in their names. For example, “first antibody” and “second antibody” are used to distinguish two antibodies.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “optional” or “optionally” means that the subsequently described circumstance may or may not occur, so that the description includes instances wherein the circumstance occurs, and the instances wherein the circumstance does not occur.

### Binding Agents—Binding Domains

In some embodiments, the present disclosure provides multispecific binding agents that can be used herein as therapeutic agents. Such agents include multispecific antibodies (e.g., antibodies, such as bispecific antibodies) comprising a first binding domain that binds to CD47, including human CD47, and a second binding domain that binds one or more additional targets that are not CD47 (e.g., PD-L1). Exemplary antibodies include humanized, human, bispecific, and heteroconjugate antibodies, as well as variants thereof having increased or decreased affinity or other properties.

In some embodiments, described herein are multispecific binding agents (e.g., antibodies, such as bispecific antibodies) comprising a first binding domain that binds to CD47, including a CD47 polypeptide, a CD47 polypeptide fragment, a CD47 peptide or a CD47 epitope. In some embodiments, the multispecific binding agents are human or humanized antibodies (e.g., comprising human constant regions) comprising a first binding domain that binds CD47, including a CD47 polypeptide, a CD47 polypeptide fragment, a CD47 peptide or a CD47 epitope. In some embodiments, a multispecific binding agent (e.g., an antibody, such as a bispecific antibody) can bind to CD47 expressed on the surface of a mammalian (e.g., human) cell, including a CD47-expressing tumor cell. In some embodiments, a multispecific binding agent (e.g., an antibody, such as a bispecific antibody) binds a CD47 extracellular epitope exposed on a cell such as a tumor cell (e.g., a CD47 epitope). In some embodiments, described herein is a multispecific binding agent (e.g., an antibody, such as a bispecific antibody) that binds to CD47, such as human CD47 or portions thereof. In some embodiments, CD47 is a human CD47. In some embodiments, provided herein is a multispecific binding agent that binds to CD47 (e.g., an antibody that binds to human CD47). An exemplary amino acid sequence of human CD47 is described herein.

Multispecific binding agents (e.g., antibodies, such as bispecific antibodies), including multispecific binding agents that bind to CD47, including human CD47, and one or more targets that are not CD47 (e.g., PD-L1, including human PD-L1), described herein may be monospecific, bispecific, trispecific or of greater multispecificity. Such agents may include antibodies. Multispecific antibodies, such as bispecific antibodies, are monoclonal antibodies that have binding specificities for at least two different targets (e.g., antigens) or two different epitopes on the same target (e.g., a bispecific antibody directed to CD47 with a first binding domain for a first epitope of a CD47, and a second binding domain for a second epitope of CD47). In some embodiments, the first binding domain of multispecific (e.g., bispecific) antibodies described herein can be constructed based on the sequences of the antibodies described herein, e.g., the CDR sequences listed in Table 1. In some embodiments, the second binding domain of multispecific (e.g., bispecific) antibodies described herein can be constructed based on the sequences of the antibodies described herein, e.g., the CDR sequences listed in Table 2. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for CD47 and the other is for any other target (e.g., antigen). In some embodiments, a multispecific (e.g., bispecific) antibody can comprise more than one target binding domain, in which different domains are specific for different targets (e.g., a first binding domain that binds CD47 and a second binding domain that binds another target (e.g., antigen), such as an immune checkpoint regulator (e.g., a negative checkpoint regulator). In some embodiments, a multispecific (e.g., bispecific) antibody can bind more than one (e.g., two or more) epitopes on the same target (e.g., antigen). In some embodiments, one of the binding specificities is for CD47 and the other is for any other target (e.g., antigen). In some embodiments, one of the binding specificities is CD47 and the other is for one or more of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092.

In some embodiments, described herein are multispecific binding agents (e.g., antibodies, such as bispecific antibodies) comprising a second binding domain that binds to PD-L1, including a PD-L1 polypeptide, a PD-L1 polypeptide fragment, a PD-L1 peptide or a PD-L1 epitope. In some embodiments, the multispecific binding agents are humanized antibodies (e.g., comprising human constant regions) comprising a second binding domain that binds PD-L1, including a PD-L1 polypeptide, a PD-L1 polypeptide fragment, a PD-L1 peptide or a PD-L1 epitope. In some embodiments, a multispecific binding agent (e.g., an antibody, such as a bispecific antibody) can bind to PD-L1 expressed on the surface of a mammalian (e.g., human) cell, including a PD-L1 expressing antigen presenting cells and tumor cells. In some embodiments, a multispecific binding agent (e.g., an antibody, such as a bispecific antibody) binds a PD-L1 extracellular epitope exposed on a cell such as a tumor cell (e.g., a PD-L1 epitope). In some embodiments, described herein is a multispecific binding agent (e.g., an antibody, such as a bispecific antibody) comprising a second binding domain that binds to PD-L1, such as human PD-L1 or portions thereof. In some embodiments, PD-L1 is a human PD-L1. In some embodiments, provided herein is a multispecific binding agent that binds to human PD-L1 (e.g., an antibody that binds to human PD-L1). An exemplary amino acid sequence of human PD-L1 is described herein.

In some embodiments, the multispecific binding agent (e.g., a bispecific antibody) provided herein binds to CD47 (e.g., human CD47) with a dissociation constant (K_{D}) of ≤1 μM, ≤500 nM, ≤100 nM, ≤50 nM, ≤30 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). Additionally or alternatively, the multispecific binding agent (e.g., a bispecific antibody) provided herein binds to CD47 (e.g., human CD47) with a dissociation constant (K_{D}) of ≥0.1 nM, ≥1 nM, ≥5 nM, ≥10 nM, ≥15 nM, or ≥ 20 nM. In further embodiments, the multispecific binding agent (e.g., a bispecific antibody) provided herein binds to CD47 (e.g., human CD47) with a dissociation constant (K_{D}) of about 0.1 nM to about 1 μM, including each number and range therebetween, such as about 1 nM to about 100 nM, or about 10 nM to about 50 nM, or about 20 nM to about 30 nM. Additionally or alternatively, the multispecific binding agent (e.g., a bispecific antibody) provided herein binds to PD-L1 (e.g., human PD-L1) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by OCTET®, using, for example, an OCTET®Red96 system, or by BIACORE®, using, for example, a BIACORE®TM-2000 or a BIACORE®TM-3000. An “on-rate” or “rate of association” or “association rate” or “kon” may also be determined with the same biolayer interferometry (BLI) or surface plasmon resonance (SPR) techniques described above using, for example, the OCTET®Red96, the BIACORE®TM-2000, the BIACORE®TM-3000 system, the BIACORE®TM-8K, or the BIACORE®TM-8K+ system.

In some embodiments, the multispecific binding agents (e.g., antibodies, such as bispecific antibodies) described herein compete for the binding to CD47, such as human CD47, with a binding agent (e.g., an antibody, such as a bispecific antibody) that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 set forth in Table 1. Accordingly, in some embodiments, a multispecific binding agent (e.g., an antibody, such as a bispecific antibody) described herein competes for the binding to CD47, such as human CD47, with a binding agent (e.g., an antibody, such as a bispecific antibody) that comprises one, two, and/or three VH CDRs and/or one, two, and/or three VL CDRs from the antibody designated mAb-C, as shown in Table 1. In some embodiments, a multispecific binding agent (e.g., an antibody, such as a bispecific antibody) described herein competes for the binding to CD47, such as human CD47, with a binding agent (e.g., an antibody, such as a bispecific antibody) that comprises one, two, and/or three VH CDRs and one, two, and/or three VL CDRs from the antibody designated mAb-C, as shown in Table 1. In some embodiments, a multispecific binding agent (e.g., an antibody, such as a bispecific antibody) described herein competes for the binding to CD47, such as human CD47, with a binding agent (e.g., an antibody, such as a bispecific antibody) that comprises a VH region and VL region from the antibody designated mAb-C, as shown in Table 1. In some embodiments, a multispecific binding agent (e.g., an antibody, such as a bispecific antibody) described herein competes for the binding to CD47, such as human CD47, with a binding agent (e.g., an antibody, such as a bispecific antibody) that comprises a VH region comprising the amino acid sequence of SEQ ID NO:25 and a VL region comprising the amino acid sequence of SEQ ID NO:26.

In some embodiments, the multispecific binding agents (e.g., antibodies, such as bispecific antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 set forth in Tables 1-2. Accordingly, in some embodiments, the multispecific binding agent (e.g., an antibody, such as a bispecific antibody) described herein comprises a first binding domain that comprises one, two, and/or three heavy chain CDRs and/or one, two, and/or three light chain CDRs from the antibody designated mAb-C, as shown in Table 1. In some embodiments, the multispecific binding agent (e.g., an antibody, such as a bispecific antibody) described herein comprises a second binding domain that comprises one, two, and/or three heavy chain CDRs and/or one, two, and/or three light chain CDRs from the antibody designated mAb-P, as shown in Table 2. In some embodiments, the multispecific binding agent (e.g., an antibody, such as a bispecific antibody) described herein comprises a first binding domain that comprises one, two, and/or three heavy chain CDRs and/or one, two, and/or three light chain CDRs from the antibody designated mAb-C, as shown in Table 1, and a second binding domain that comprises one, two, and/or three heavy chain CDRs and/or one, two, and/or three light chain CDRs from the antibody designated mAb-P, as shown in Table 2. In some embodiments, the multispecific binding agent (e.g., an antibody, such as a bispecific antibody) described herein comprises a first binding domain that comprises one, two, and/or three heavy chain CDRs and one, two, and/or three light chain CDRs from the antibody designated mAb-C, as shown in Table 1, and a second binding domain that comprises one, two, and/or three heavy chain CDRs and one, two, and/or three light chain CDRs from the antibody designated mAb-P, as shown in Table 2.

In some embodiments, a multispecific binding agent (e.g., an antibody, such as a bispecific antibody) comprises a first binding domain that binds to CD47 and comprises a VH region, which comprises VH CDR1, VH CDR2, and/or VH CDR3, and a VL region, which comprises VL CDR1, VL CDR2, and/or VL CDR3, of any one of the binding agents described in Table 1, and a second binding domain that binds to PD-L1 and comprises a VH region, which comprises VH CDR1, VH CDR2, and/or VH CDR3, and a VL region, which comprises VL CDR1, VL CDR2, and/or VL CDR3, of any one of the binding agents described in Table 2.

Accordingly, in some embodiments, a multispecific binding agent (e.g., an antibody, such as a bispecific antibody) described herein comprises a first binding domain that comprises one, two, and/or three heavy chain CDRs and/or one, two, and/or three light chain CDRs from Table 1. In some embodiments, the multispecific binding agent (e.g., an antibody, such as a bispecific antibody) described herein comprises a second binding domain that comprises one, two, and/or three heavy chain CDRs and/or one, two, and/or three light chain CDRs from Table 2. In some embodiments, a multispecific binding agent (e.g., an antibody, such as a bispecific antibody) described herein is bispecific and comprises a first binding domain that comprises one, two, and/or three heavy chain CDRs and/or one, two, and/or three light chain CDRs from Table 1 and a second binding domain that comprises one, two, and/or three heavy chain CDRs and/or one, two, and/or three light chain CDRs from a binding agent that binds to a second target antigen that is not CD47. In some embodiments, a multispecific binding agent (e.g., an antibody, such as a bispecific antibody) described herein is bispecific and comprises a first binding domain that comprises one, two, and/or three heavy chain CDRs and one, two, and/or three light chain CDRs from Table 1, and a second binding domain that comprises one, two, and/or three heavy chain CDRs and one, two, and/or three light chain CDRs from Table 2.

The antibody designated mAb-C comprises a VH sequence that is SEQ ID NO: 25 and a VL sequence that is SEQ ID NO:26.

The antibody designated mAb-P comprises a VH sequence that is SEQ ID NO: 46 and a VL sequence that is SEQ ID NO:47.

In some embodiments, a multispecific binding agent (e.g., an antibody) that binds to CD47 comprises (i) a VH domain wherein the VH domain comprises a VH sequence that is SEQ ID NO:25 and (ii) a VL domain wherein the VL domain comprises a VL sequence that is SEQ ID NO:26. In some embodiments, such VH and VL domains were used to construct bispecific binding agents (e.g., antibodies) each with a first binding domain that bind to CD47, including wherein the first binding domain comprises a VH sequence that is SEQ ID NO:26 and a VL sequence that is SEQ ID NO:26.

In some embodiments, a multispecific binding agent (e.g., an antibody) that binds to PD-L1 comprises (i) a VH domain wherein the VH domain comprises a VH sequence that is SEQ ID NO:46 and (ii) a VL domain wherein the VL domain comprises a VL sequence that is SEQ ID NO:47. In some embodiments, such VH and VL domains were used to construct bispecific binding agents (e.g., antibodies) each with a first binding domain that bind to PD-L1, including wherein the first binding domain comprises a VH sequence that is SEQ ID NO:46 and a VL sequence that is SEQ ID NO:47.

In some embodiments, a multispecific binding agent (e.g., a bispecific antibody) that binds to CD47 and PD-L1 comprises (a) a first binding domain comprising (i) a VH domain wherein the VH domain comprises a VH sequence that is SEQ ID NO:25 and (ii) a VL domain wherein the VL domain comprises a VL sequence that is SEQ ID NO:26, and (b) a second binding domain comprising (i) a VH domain wherein the VH domain comprises a VH sequence that is SEQ ID NO:46 and (ii) a VL domain wherein the VL domain comprises a VL sequence that is SEQ ID NO: 47.

In some embodiments, such VH and VL domains were used to construct bispecific antibodies comprising a first polypeptide chain that comprises a VL domain (e.g., a VL sequence that is SEQ ID NO:26), a second polypeptide chain that comprises a VH domain (e.g., a VH sequence that is SEQ ID NO:25), wherein the VL and VH domains form a first binding domain that binds to CD47 and further comprising a third polypeptide chain that comprises a VL domain (e.g., a VL sequence that is SEQ ID NO:47), a fourth polypeptide chain that comprises a VH domain (e.g., a VH sequence that is SEQ ID NO:46), wherein the VL and VH domains form a second binding domain that binds to PD-L1.

In an exemplary embodiment of a multispecific binding agent (e.g., a bispecific antibody) comprising four polypeptide chains, a first polypeptide chain having an amino acid sequence of SEQ ID NO:48, a second polypeptide chain having an amino acid sequence of SEQ ID NO:49, a third polypeptide chain having an amino acid sequence of SEQ ID NO:50, and a fourth polypeptide chain having an amino acid sequence of SEQ ID NO:51. In another exemplary embodiment of a multispecific binding agent (e.g., a bispecific antibody) comprising four polypeptide chains, a first polypeptide chain having an amino acid sequence of SEQ ID NO:52, a second polypeptide chain having an amino acid sequence of SEQ ID NO:49, a third polypeptide chain having an amino acid sequence of SEQ ID NO:53, and a fourth polypeptide chain having an amino acid sequence of SEQ ID NO:51.

TABLE 1
Antibody mAb-C

| | | Exemplary | IMGT | Kabat | Chothia | Contact | AbM |
|---|---|---|---|---|---|---|---|
| VH | VH | GFTFSWYYIH | GFTFSWYY | WYYIH | GFTFSWY | SWYYIH | GFTFSWYYIH |
| CDR | CDR1 | (SEQ ID NO: 1) | (SEQ ID NO: 7) | (SEQ ID NO: 12) | (SEQ ID NO: 13) | (SEQ ID NO: 18) | (SEQ ID NO: 1) |
| Seq. | VH | YIDSSSGYTEYADS | IDSSSGYT | YIDSSSGYTEYADS | SSSG | WVAYIDSSSGYTE | YIDSSSGYTE |
| | CDR2 | VKG | (SEQ ID NO: 8) | VKG | (SEQ ID NO: 14) | (SEQ ID NO: 19) | (SEQ ID NO: 24) |
| | | (SEQ ID NO: 2) | | (SEQ ID NO: 2) | | | |
| | VH | GGYLGMDY | ARGGYLGMDY | GGYLGMDY | GYLGMD | ARGGYLGMD | GGYLGMDY |
| | CDR3 | (SEQ ID NO: 3) | (SEQ ID NO: 9) | (SEQ ID NO: 3) | (SEQ ID NO: 15) | (SEQ ID NO: 20) | (SEQ ID NO: 3) |
| VL | VL | RASQSVSSAVA | QSVSSA | RASQSVSSAVA | SQSVSSA | SSAVAWY | RASQSVSSAVA |
| CDR | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| Seq. | VL | SASSLYS | SAS | SASSLYS | SAS | LLIYSASSLY | SASSLYS |
| | CDR2 | (SEQ ID NO: 5) | (SEQ ID NO: 11) | (SEQ ID NO: 5) | (SEQ ID NO: 11) | (SEQ ID NO: 22) | (SEQ ID NO: 5) |
| | VL | QQSRSRPRT | QQSRSRPRT | QQSRSRPRT | SRSRPR | QQSRSRPR | QQSRSRPRT |
| | CDR3 | (SEQ ID NO: 6) | (SEQ ID NO: 6) | (SEQ ID NO: 6) | (SEQ ID NO: 17) | (SEQ ID NO: 23) | (SEQ ID NO: 6) |

VH Sequence:
EVQLVESGGGLVQPGGSLRLSCAASGFTFSWYYIHWVRQAPGKGLEWVAYIDSSSGYTEYADSVKGRFTISADTSKNTAYLQMNSLRAEDT
AVYYCARGGYLGMDYWGQGTLVTVSS
(SEQ ID NO: 25)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQS
RSRPRTFGQGTKVEIK
(SEQ ID NO: 26)

TABLE 2
Antibody mAb-P

| | | Exemplary | IMGT | Kabat | Chothia | Contact | AbM |
|---|---|---|---|---|---|---|---|
| VH | VH | GFTFRSYYIH | GFTFRSYY | SYYIH | GFTFRSY | RSYYIH | GFTFRSYYIH |
| CDR | CDR1 | (SEQ ID NO: 27) | (SEQ ID NO: 32) | (SEQ ID NO: 35) | (SEQ ID NO: 36) | (SEQ ID NO: 40) | (SEQ ID NO: 27) |
| Seq. | VH | TISSYGVFTYYADS | ISSYGVFT | TISSYGVFTYYADS | SYGV | WVATISSYGVFTY | TISSYGVFTY |
| | CDR2 | VKG | (SEQ ID NO: 33) | VKG | (SEQ ID NO: 37) | (SEQ ID NO: 41) | (SEQ ID NO: 45) |
| | | (SEQ ID NO: 28) | | (SEQ ID NO: 28) | | | |
| | VH | GTTLTPVLDY | ARGTTLTPVLDY | GTTLTPVLDY | TTLTPVLD | ARGTTLTPVLD | GTTLTPVLDY |
| | CDR3 | (SEQ ID NO: 29) | (SEQ ID NO: 34) | (SEQ ID NO: 29) | (SEQ ID NO: 38) | (SEQ ID NO: 42) | (SEQ ID NO: 29) |
| VL | VL | RASQSVSSAVA | QSVSSA | RASQSVSSAVA | SQSVSSA | SSAVAWY | RASQSVSSAVA |
| CDR | CDR1 | (SEQ ID NO: 4) | (SEQ ID NO: 10) | (SEQ ID NO: 4) | (SEQ ID NO: 16) | (SEQ ID NO: 21) | (SEQ ID NO: 4) |
| Seq. | VL | SASSLSS | SAS | SASSLSS | SAS | LLIYSASSLS | SASSLSS |
| | CDR2 | (SEQ ID NO: 30) | (SEQ ID NO: 11) | (SEQ ID NO: 30) | (SEQ ID NO: 11) | (SEQ ID NO: 43) | (SEQ ID NO: 30) |
| | VL | QQFGAEPIT | QQFGAEPIT | QQFGAEPIT | FGAEPI | QQFGAEPI | QQFGAEPIT |
| | CDR3 | (SEQ ID NO: 31) | (SEQ ID NO: 31) | (SEQ ID NO: 31) | (SEQ ID NO: 39) | (SEQ ID NO: 44) | (SEQ ID NO: 31) |

VH Sequence:
EVQLVESGGGLVQPGGSLRLSCAASGFTFRSYYIHWVRQAPGKGLEWVATISSYGVFTYYADSVKGRFTISADTSKNTAYLQMNSLRAEDTA
VYYCARGTTLTPVLDYWGQGTLVTVSS (SEQ ID NO: 46)
VL Sequence:
DIQMTQSPSSLSASVGDRVTITCRASQSVSSAVAWYQQKPGKAPKLLIYSASSLSSGVPSRFSGSRSGTDFTLTISSLQPEDFATYYCQQFG
AEPITFGQGTKVEIK (SEQ ID NO: 47)

In some embodiments, multispecific binding agents (e.g., antibodies, such as bispecific antibodies) described herein comprise a first binding domain that binds to CD47, including human CD47, and a second binding domain that binds to one or more targets that are not CD47 (e.g., PD-L1, including human PD-L1), wherein the first binding domain and/or the second binding domain comprise a VH region or VH domain. In other embodiments, multispecific binding agents (e.g., antibodies, such as bispecific antibodies) described herein comprise a first binding domain that binds to CD47, including human CD47, and a second binding domain that binds to one or more targets that are not CD47 (e.g., PD-L1, including human PD-L1), wherein the first binding domain and/or the second binding domain comprise a VL region or VL domain. In some embodiments, multispecific binding agents (e.g., antibodies, such as bispecific antibodies) described herein comprise a first binding domain that binds to CD47, including human CD47, and a second binding domain that binds to one or more targets that are not CD47 (e.g., PD-L1, including human PD-L1), wherein the first binding domain and/or the second binding domain have a combination of (i) a VH domain or VH region; and/or (ii) a VL domain or VL region.

In some embodiments, multispecific binding agents (e.g., antibodies, such as bispecific antibodies) described herein comprise a first binding domain that binds to CD47, including human CD47, and a second binding domain that binds one or more targets that are not CD47 (e.g., PD-L1, including human PD-L1), wherein the first binding domain comprises one or more CDRs, including six CDRs, for example, VH CDR1, VH CDR2, VH CDR3, VL CDR1, VL CDR2, and/or VL CDR3 identified in Table 1.

In some embodiments, multispecific binding agents (e.g., antibodies, such as bispecific antibodies), including a multispecific binding agent that binds to CD47, including human CD47, and one or more targets that are not CD47 (e.g., PD-L1, including human PD-L1), described herein comprise a first binding domain comprising one or more CDRs, including three VH CDRs, for example, VH CDR1, VH CDR2, VH CDR3, listed in Table 1. In other embodiments, multispecific binding agents (e.g., antibodies, such as bispecific antibodies), including multispecific binding agents that bind to CD47, including human CD47, and one or more targets that are not CD47 (e.g., PD-L1, including human PD-L1), described herein comprise a first binding domain comprising one or more CDRs, including three CDRs, for example, VL CDR1, VL CDR2, and/or VL CDR3, listed in Table 1. In yet other embodiments, multispecific binding agents (e.g., antibodies, such as bispecific antibodies), including multispecific binding agents that bind to CD47, including human CD47, and one or more targets that are not CD47 (e.g., PD-L1, including human PD-L1), described herein comprise a first binding domain comprising one or more CDRs, including three VH CDRs, for example, VH CDR1, VH CDR2, VH CDR3, listed in Table 1 and one or more CDRs, including three VL CDRs, for example, VL CDR1, VL CDR2, and/or VL CDR3, listed in Table 1.

In some embodiments, multispecific binding agents (e.g., antibodies, such as bispecific antibodies), including a multispecific binding agent that binds to CD47, including human CD47, and PD-L1, including human PD-L1, described herein comprise a second binding domain comprising one or more CDRs, including three VH CDRs, for example, VH CDR1, VH CDR2, VH CDR3, listed in Table 2. In other embodiments, multispecific binding agents (e.g., antibodies, such as bispecific antibodies), including multispecific binding agents that bind to CD47, including human CD47, and PD-L1, including human PD-L1, described herein comprise a second binding domain comprising one or more CDRs, including three CDRs, for example, VL CDR1, VL CDR2, and/or VL CDR3, listed in Table 2. In yet other embodiments, multispecific binding agents (e.g., antibodies, such as bispecific antibodies), including multispecific binding agents that bind to CD47, including human CD47, and PD-L1, including human PD-L1, described herein comprise a second binding domain comprising one or more CDRs, including three VH CDRs, for example, VH CDR1, VH CDR2, VH CDR3, listed in Table 2 and one or more CDRs, including three VL CDRs, for example, VL CDR1, VL CDR2, and/or VL CDR3, listed in Table 2.

In some embodiments, a multispecific binding agent (e.g., an antibody, such as a bispecific antibody) described herein comprises a first binding domain that binds to CD47 and comprises one or more complementarity determining regions (CDRs) comprising an amino acid sequence selected from a group consisting of SEQ ID NOS: 1-24. In some embodiments, a multispecific binding agent (e.g., an antibody, such as a bispecific antibody) described herein comprises a first binding domain that binds to CD47 and comprises two or more complementarity determining regions (CDRs) comprising an amino acid sequence selected from a group consisting of SEQ ID NOS: 1-24. In some embodiments, a multispecific binding agent (e.g., an antibody, such as a bispecific antibody) described herein comprises a first binding domain that binds to CD47 and comprises three or more complementarity determining regions (CDRs) comprising an amino acid sequence selected from a group consisting of SEQ ID NOS: 1-24. In some embodiments, the multispecific binding agent (e.g., an antibody, such as a bispecific antibody) described herein comprises a second binding domain that binds to PD-L1 and comprises one or more complementarity determining regions (CDRs) comprising an amino acid sequence selected from a group consisting of SEQ ID NOS: 27-45. In some embodiments, a multispecific binding agent (e.g., an antibody, such as a bispecific antibody) described herein comprises a second binding domain that binds to PD-L1 and comprises two or more complementarity determining regions (CDRs) comprising an amino acid sequence selected from a group consisting of SEQ ID NOS: 27-45. In some embodiments, a multispecific binding agent (e.g., an antibody, such as a bispecific antibody) described herein comprises a second binding domain that binds to PD-L1 and comprises three or more complementarity determining regions (CDRs) comprising an amino acid sequence selected from a group consisting of SEQ ID NOS: 27-45.

In some embodiments, a multispecific binding agent (e.g., an antibody, such as a bispecific antibody) described herein comprises a first binding domain that binds to CD47 and comprises a VH with one or more (e.g., one, two or three) VH CDRs listed in Table 1. In other embodiments, a multispecific binding agent (e.g., an antibody, such as a bispecific antibody) described herein comprises a first binding domain that binds to CD47 and comprises a VL with one or more (e.g., one, two or three) VL CDRs listed in Table 1. In yet other embodiments, the multispecific binding agent (e.g., an antibody, such as a bispecific antibody) described herein comprises a first binding domain that binds to CD47 and comprises one or more (e.g., one, two or three) VH CDRs listed in Table 1 and one or more VL CDRs listed in Table 1. Accordingly, in some embodiments, a multispecific binding agent (e.g., an antibody, such as a bispecific antibody) described herein comprises a first binding domain that binds to CD47 and comprises a VH CDR1 having the amino acid sequence of any one of SEQ ID NOS: 1, 7, 12, 13, and 18. In some embodiments, a multispecific binding agent (e.g., an antibody, such as a bispecific antibody) described herein comprises a first binding domain that binds to CD47 and comprises a VH CDR2 having the amino acid sequence of any one of SEQ ID NOS: 2, 8, 14, 19, and 24. In some embodiments, a multispecific binding agent (e.g., an antibody, such as a bispecific antibody) described herein a first binding domain that binds to CD47 and comprises a VH CDR3 having the amino acid sequence of any one of SEQ ID NOS: 3, 9, 15, and 20. In some embodiments, a multispecific binding agent (e.g., an antibody, such as a bispecific antibody) described herein comprises a first binding domain that binds to CD47 and comprises a VH CDR1 and/or a VH CDR2 and/or a VH CDR3 independently selected from a VH CDR1, VH CDR2, VH CDR3 as set forth in any one of the amino acid sequences set forth in Table 1. In some embodiments, a multispecific binding agent (e.g., an antibody, such as a bispecific antibody) described herein comprises a first binding domain that binds to CD47 and comprises a VL CDR1 having the amino acid sequence of any one of SEQ ID NOS: 4, 10, 16, and 21. In another embodiment, a multispecific binding agent (e.g., an antibody, such as a bispecific antibody) described herein comprises a first binding domain that binds to CD47 and comprises a VL CDR2 having the amino acid sequence of any one of SEQ ID NOS: 5, 11, and 22. In some embodiments, a multispecific binding agent (e.g., an antibody, such as a bispecific antibody) described herein comprises a first binding domain that binds to CD47 and comprises a VL CDR3 having the amino acid sequence of any one of SEQ ID NOS: 6, 17, and 23. In some embodiments, a multispecific binding agent (e.g., an antibody, such as a bispecific antibody) described herein comprises a first binding domain that binds to CD47 and comprises a VL CDR1 and/or a VL CDR2 and/or a VL CDR3 independently selected from a VL CDR1, VL CDR2, VL CDR3 as set forth in any one of the amino acid sequences set forth in Table 1.

In some embodiments, a multispecific binding agent (e.g., an antibody, such as a bispecific antibody) described herein comprises a second binding domain that binds to PD-L1 and comprises a VH with one or more (e.g., one, two or three) VH CDRs listed in Table 2. In other embodiments, a multispecific binding agent (e.g., an antibody, such as a bispecific antibody) described herein comprises a second binding domain that binds to PD-L1 and comprises a VL with one or more (e.g., one, two or three) VL CDRs listed in Table 2. In yet other embodiments, the multispecific binding agent (e.g., an antibody, such as a bispecific antibody) described herein comprises a second binding domain that binds to PD-L1 and comprises one or more (e.g., one, two or three) VH CDRs listed in Table 2 and one or more VL CDRs listed in Table 2. Accordingly, in some embodiments, a multispecific binding agent (e.g., an antibody, such as a bispecific antibody) described herein comprises a second binding domain that binds to PD-L1 and comprises a VH CDR1 having the amino acid sequence of any one of SEQ ID NOS: 27, 32, 35, 36, and 40. In some embodiments, a multispecific binding agent (e.g., an antibody, such as a bispecific antibody) described herein comprises a second binding domain that binds to PD-L1 and comprises a VH CDR2 having the amino acid sequence of any one of SEQ ID NOS: 28, 33, 37, 41, and 45. In some embodiments, a multispecific binding agent (e.g., an antibody, such as a bispecific antibody) described herein comprises a second binding domain that binds to PD-L1 and comprises a VH CDR3 having the amino acid sequence of any one of SEQ ID NOS: 29, 34, 38, and 42. In some embodiments, a multispecific binding agent (e.g., an antibody, such as a bispecific antibody) described herein comprises a second binding domain that binds to PD-L1 and comprises a VH CDR1 and/or a VH CDR2 and/or a VH CDR3 independently selected from a VH CDR1, VH CDR2, VH CDR3 as set forth in any one of the amino acid sequences set forth in Table 2. In some embodiments, a multispecific binding agent (e.g., an antibody, such as a bispecific antibody) described herein comprises a second binding domain that binds to PD-L1 and comprises a VL CDR1 having the amino acid sequence of any one of SEQ ID NOS: 4, 10, 16, and 21. In another embodiment, a multispecific binding agent (e.g., an antibody, such as a bispecific antibody) described herein comprises a second binding domain that binds to PD-L1 and comprises a VL CDR2 having the amino acid sequence of any one of SEQ ID NOS: 11, 30, and 43. In some embodiments, a multispecific binding agent (e.g., an antibody, such as a bispecific antibody) described herein comprises a second binding domain that binds to PD-L1 and comprises a VL CDR3 having the amino acid sequence of any one of SEQ ID NOS: 31, 39, and 44. In some embodiments, a multispecific binding agent (e.g., an antibody, such as a bispecific antibody) described herein comprises a second binding domain that binds to PD-L1 and comprises a VL CDR1 and/or a VL CDR2 and/or a VL CDR3 independently selected from a VL CDR1, VL CDR2, VL CDR3 as set forth in any one of the amino acid sequences set forth in Table 2.

In some embodiments, a multispecific binding agent (e.g., an antibody, such as a bispecific antibody) described herein comprises a first binding domain that binds to CD47 and comprises a heavy chain variable (VH) region comprising: (1) a VH CDR1 having an amino acid sequence of selected from the group consisting of: (i) SEQ ID NO:1, (ii) SEQ ID NO:7, (iii) SEQ ID NO:12, (iv) SEQ ID NO:13, and (v) SEQ ID NO: 18; (2) a VH CDR2 having an amino acid sequence of selected from the group consisting of: (i) SEQ ID NO:2, (ii) SEQ ID NO:8, (iii) SEQ ID NO:14, (iv) SEQ ID NO: 19, and (v) SEQ ID NO:24; and (3) a VH CDR3 having an amino acid sequence of selected from the group consisting of: (i) SEQ ID NO:3, (ii) SEQ ID NO: 9, (iii) SEQ ID NO: 15, and (iv) SEQ ID NO:20; and/or a light chain variable (VL) region comprising: (1) a VL CDR1 having an amino acid sequence of selected from the group consisting of: (i) SEQ ID NO:4, (ii) SEQ ID NO: 10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO:6, (ii) SEQ ID NO: 17, and (iii) SEQ ID NO:23.

In some embodiments, a multispecific binding agent (e.g., an antibody, such as a bispecific antibody) described herein comprises a first binding domain that binds to CD47 and comprises a heavy chain variable (VH) region comprising: (1) a VH CDR1 having an amino acid sequence of selected from the group consisting of: (i) SEQ ID NO:1, (ii) SEQ ID NO:7, (iii) SEQ ID NO:12, (iv) SEQ ID NO:13, and (v) SEQ ID NO: 18; (2) a VH CDR2 having an amino acid sequence of selected from the group consisting of: (i) SEQ ID NO:2, (ii) SEQ ID NO:8, (iii) SEQ ID NO: 14, (iv) SEQ ID NO: 19, and (v) SEQ ID NO:24; and (3) a VH CDR3 having an amino acid sequence of selected from the group consisting of: (i) SEQ ID NO:3, (ii) SEQ ID NO: 9, (iii) SEQ ID NO:15, and (iv) SEQ ID NO:20.

In some embodiments, a multispecific binding agent (e.g., an antibody, such as a bispecific antibody) described herein comprises a first binding domain that binds to CD47 and comprises a light chain variable (VL) region comprising: (1) a VL CDR1 having an amino acid sequence of selected from the group consisting of: (i) SEQ ID NO:4, (ii) SEQ ID NO: 10, (iii) SEQ ID NO:16, and (iv) SEQ ID NO:21; (2) a VL CDR2 having an amino acid sequence of selected from the group consisting of: (i) SEQ ID NO:5, (ii) SEQ ID NO:11, and (iii) SEQ ID NO:22; and (3) a VL CDR3 having an amino acid sequence of selected from the group consisting of: (i) SEQ ID NO: 6, (ii) SEQ ID NO: 17, and (iii) SEQ ID NO:23.

In some embodiments, a multispecific binding agent (e.g., an antibody, such as a bispecific antibody) described herein comprises a second binding domain that binds to PD-L1 and comprises (a) a heavy chain variable (VH) region comprising (1) a VH CDR1 having an amino acid sequence selected from the group consisting of: (i) SEQ ID NO:27, (ii) SEQ ID NO:32, (iii) SEQ ID NO:35, (iv) SEQ ID NO:36 (v) SEQ ID NO:40; (2) a VH CDR2 having an amino acid sequence selected from the group consisting of: (i) SEQ ID NO:28, (ii) SEQ ID NO:33, (iii) SEQ ID NO:37, (iv) SEQ ID NO:41, (v) SEQ ID NO:45; and (3) a VH CDR3 having an amino acid sequence selected from the group consisting of: (i) SEQ ID NO:29, (ii) SEQ ID NO: 34, (iii) SEQ ID NO:38, (iv) SEQ ID NO:42; and/or (b) a light chain variable (VL) region comprising: (1) a VL CDR1 having an amino acid sequence selected from the group consisting of: (i) SEQ ID NO:4, (ii) SEQ ID NO: 10, (iii) SEQ ID NO: 16, (iv) SEQ ID NO:21; (2) a VL CDR2 having an amino acid sequence selected from the group consisting of: (i) SEQ ID NO:30, (ii) SEQ ID NO:11, (iii) SEQ ID NO:43; (3) a VL CDR3 having an amino acid sequence selected from the group consisting of: (i) SEQ ID NO:31, (ii) SEQ ID NO:39, (iii) SEQ ID NO:44.

In some embodiments, a multispecific binding agent (e.g., an antibody, such as a bispecific antibody) described herein comprises a second binding domain that binds to PD-L1 and comprises a heavy chain variable (VH) region comprising (1) a VH CDR1 having an amino acid sequence selected from the group consisting of: (i) SEQ ID NO:27, (ii) SEQ ID NO:32, (iii) SEQ ID NO:35, (iv) SEQ ID NO:36 (v) SEQ ID NO: 40; (2) a VH CDR2 having an amino acid sequence selected from the group consisting of: (i) SEQ ID NO:28, (ii) SEQ ID NO:33, (iii) SEQ ID NO:37, (iv) SEQ ID NO: 41, (v) SEQ ID NO:45; and (3) a VH CDR3 having an amino acid sequence selected from the group consisting of: (i) SEQ ID NO:29, (ii) SEQ ID NO:34, (iii) SEQ ID NO: 38, (iv) SEQ ID NO:42.

In some embodiments, a multispecific binding agent (e.g., an antibody, such as a bispecific antibody) described herein comprises a second binding domain that binds to PD-L1 and comprises a light chain variable (VL) region comprising: (1) a VL CDR1 having an amino acid sequence selected from the group consisting of: (i) SEQ ID NO:4, (ii) SEQ ID NO: 10, (iii) SEQ ID NO:16, (iv) SEQ ID NO:21; (2) a VL CDR2 having an amino acid sequence selected from the group consisting of: (i) SEQ ID NO: 30, (ii) SEQ ID NO: 11, (iii) SEQ ID NO:43; (3) a VL CDR3 having an amino acid sequence selected from the group consisting of: (i) SEQ ID NO:31, (ii) SEQ ID NO: 39, (iii) SEQ ID NO:44.

Also described herein are multispecific binding agents (e.g., antibodies, such as bispecific antibodies) comprising a first binding domain that binds to CD47 and comprises one or more (e.g., one, two or three) VH CDRs and one or more (e.g., one, two or three) VL CDRs listed in Table 1. In particular, described herein is a multispecific binding agent (e.g., an antibody, such as a bispecific antibody) comprising: a VH CDR1 (SEQ ID NOS: 1, 7, 12, 13, and 18) and a VL CDR1 (SEQ ID NOS: 4, 10, 16, and 21); a VH CDR1 (SEQ ID NOS: 1, 7, 12, 13, and 18) and a VL CDR2 (SEQ ID NOS: 5, 11, and 22); a VH CDR1 (SEQ ID NOS: 1, 7, 12, 13, and 18) and a VL CDR3 (SEQ ID NOS: 6, 17, and 23); a VH CDR2 (SEQ ID NOS: 2, 8, 14, 19, and 24) and a VL CDR1 (SEQ ID NOS: 4, 10, 16, and 21); a VH CDR2 (SEQ ID NOS: 2, 8, 14, 19, and 24) and a VL CDR2 (SEQ ID NOS: 5, 11, and 22); a VH CDR2 (SEQ ID NOS: 2, 8, 14, 19, and 24) and a VL CDR3 (SEQ ID NOS: 6, 17, and 23); a VH CDR3 (SEQ ID NOS: 3, 9, 15, and 20) and a VL CDR1 (SEQ ID NOS: 4, 10, 16, and 21); a VH CDR3 (SEQ ID NOS: 3, 9, 15, and 20) and a VL CDR2 (SEQ ID NOS: 5, 11, and 22); a VH CDR3 (SEQ ID NOS: 3, 9, 15, and 20) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24) and a VL CDR1 (SEQ ID NOS: 4, 10, 16, and 21); a VH CDR1 (SEQ ID NOS: 1, 7, 12, 13, and 18), a VH CDR2 (SEQ ID NOS: 2, 8, 14, 19, and 24) and a VL CDR2 (SEQ ID NOS: 5, 11, and 22); a VH CDR1 (SEQ ID NOS: 1, 7, 12, 13, and 18), a VH CDR2 (SEQ ID NOS: 2, 8, 14, 19, and 24) and a VL CDR3 (SEQ ID NOS: 6, 17, and 23); a VH CDR2 (SEQ ID NOS: 2, 8, 14, 19, and 24), a VH CDR3 (SEQ ID NOS: 3, 9, 15, and 20) and a VL CDR1 (SEQ ID NOS: 4, 10, 16, and 21); a VH CDR2 (SEQ ID NOS: 2, 8, 14, 19, and 24), a VH CDR3 (SEQ ID NOS: 3, 9, 15, and 20) and a VL CDR2 (SEQ ID NOS: 5, 11, and 22); a VH CDR2 (SEQ ID NOS: 2, 8, 14, 19, and 24), a VH CDR3 (SEQ ID NOS: 3, 9, 15, and 20) and a VL CDR3 (SEQ ID NOS: 6, 17, and 23); a VH CDR1 (SEQ ID NOS: 1, 7, 12, 13, and 18), a VL CDR1 (SEQ ID NOS: 4, 10, 16, and 21) and a VL CDR2 (SEQ ID NOS: 5, 11, and 22); a VH CDR1 (SEQ ID NOS: 1, 7, 12, 13, and 18), a VL CDR1 (SEQ ID NOS: 4, 10, 16, and 21) and a VL CDR3 (SEQ ID NOS: 6, 17, and 23); a VH CDR1 (SEQ ID NOS: 1, 7, 12, 13, and 18), a VL CDR2 (SEQ ID NOS: 5, 11, and 22) and a VL CDR3 (SEQ ID NOS: 6, 17, and 23); a VH CDR2 (SEQ ID NOS: 2, 8, 14, 19, and 24), a VL CDR1 (SEQ ID NOS: 4, 10, 16, and 21) and a VL CDR2 (SEQ ID NOS: 5, 11, and 22); a VH CDR2 (SEQ ID NOS: 2, 8, 14, 19, and 24), a VL CDR1 (SEQ ID NOS: 4, 10, 16, and 21) and a VL CDR3 (SEQ ID NOS: 6, 17, and 23); a VH CDR2 (SEQ ID NOS: 2, 8, 14, 19, and 24), a VL CDR2 (SEQ ID NOS: 5, 11, and 22) and a VL CDR3 (SEQ ID NOS: 6, 17, and 23); a VH CDR3 (SEQ ID NOS: 3, 9, 15, and 20), a VL CDR1 (SEQ ID NOS: 4, 10, 16, and 21) and a VL CDR2 (SEQ ID NOS: 5, 11, and 22); a VH CDR3 (SEQ ID NOS: 3, 9, 15, and 20), a VL CDR1 (SEQ ID NOS: 4, 10, 16, and 21) and a VL CDR3 (SEQ ID NOS: 6, 17, and 23); a VH CDR3 (SEQ ID NOS: 3, 9, 15, and 20), a VL CDR2 (SEQ ID NOS: 5, 11, and 22) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20) and a VL CDR1 (SEQ ID NOS: 4, 10, 16, and 21); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20) and a VL CDR2 (SEQ ID NOS: 5, 11, and 22); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VL CDR1 (SEQ ID NOS: 4, 10, 16, and 21) and a VL CDR2 (SEQ ID NOS: 5, 11, and 22); a VH CDR1 (SEQ ID NOS: 1, 7, 12, 13, and 18), a VH CDR2 (SEQ ID NOS: 2, 8, 14, 19, and 24), a VL CDR1 (SEQ ID NOS: 4, 10, 16, and 21) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VL CDR2 (SEQ ID NOS: 5, 11, and 22) and a VL CDR3 (SEQ ID NOS: 6, 17, and 23); a VH CDR1 (SEQ ID NOS: 1, 7, 12, 13, and 18), a VH CDR3 (SEQ ID NOS: 3, 9, 15, and 20), a VL CDR1 (SEQ ID NOS: 4, 10, 16, and 21) and a VL CDR2 (SEQ ID NOS: 5, 11, and 22); a VH CDR1 (SEQ ID NOS: 1, 7, 12, 13, and 18), a VH CDR3 (SEQ ID NOS: 3, 9, 15, and 20), a VL CDR1 (SEQ ID NOS: 4, 10, 16, and 21) and a VL CDR3 (SEQ ID NOS: 6, 17, and 23); a VH CDR1 (SEQ ID NOS: 1, 7, 12, 13, and 18), a VH CDR3 (SEQ ID NOS: 3, 9, 15, and 20), a VL CDR2 (SEQ ID NOS: 5, 11, and 22) and a VL CDR3 (SEQ ID NOS: 6, 17, and 23); a VH CDR2 (SEQ ID NOS: 2, 8, 14, 19, and 24), a VH CDR3 (SEQ ID NOS: 3, 9, 15, and 20), a VL CDR1 (SEQ ID NOS: 4, 10, 16, and 21) and a VL CDR2 (SEQ ID NOS: 5, 11, and 22); a VH CDR2 (SEQ ID NOS: 2, 8, 14, 19, and 24), a VH CDR3 (SEQ ID NOS: 3, 9, 15, and 20), a VL CDR1 (SEQ ID NOS: 4, 10, 16, and 21) and a VL CDR3 (SEQ ID NOS: 6, 17, and 23); a VH CDR2 (SEQ ID NOS: 2, 8, 14, 19, and 24), a VH CDR3 (SEQ ID NOS: 3, 9, 15, and 20), a VL CDR2 (SEQ ID NOS: 5, 11, and 22) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20), a VL CDR1 (SEQ ID NOS: 4, 10, 16, and 21) and a VL CDR2 (SEQ ID NOS: 5, 11, and 22); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20), a VL CDR1 (SEQ ID NOS: 4, 10, 16, and 21) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VH CDR3 (SEQ ID NOS: 3, 9, 15, and 20), a VL CDR2 (SEQ ID NOS: 5, 11, and 22) and a VL CDR3 (SEQ ID NOS: 6, 17, and 23); a VH CDR1 (SEQ ID NOS: 1, 7, 12, 13, and 18), a VH CDR2 (SEQ ID NOS: 2, 8, 14, 19, and 24), a VL CDR1 (SEQ ID NOS: 4, 10, 16, and 21), a VL CDR2 (SEQ ID NOS: 5, 11, and 22), and a VL CDR3 (SEQ ID NOS: 6, 17, and 23); a VH CDR1 (SEQ ID NOS: 1, 7, 12, 13, and 18), a VH CDR3 (SEQ ID NOS: 3, 9, 15, and 20), a VL CDR1 (SEQ ID NOS: 4, 10, 16, and 21), a VL CDR2 (SEQ ID NOS: 5, 11, and 22), and a VL CDR3 (SEQ ID NOS: 6, 17, and 23); a VH CDR2 (SEQ ID NOS: 2, 8, 14, 19, and 24), a VH CDR3 (SEQ ID NOS: 3, 9, 15, and 20), a VL CDR1 (SEQ ID NOS: 4, 10, 16, and 21), a VL CDR2 (SEQ ID NOS: 5, 11, and 22), and a VL CDR3 (SEQ ID NOS: 6, 17, and 23); or any combination thereof of the VH CDRs (SEQ ID NOS: 1, 2, 3, 7, 8, 9, 12, 13, 14, 15, 18, 19, 20, and 24) and VL CDRs (SEQ ID NOS: 4, 5, 6, 10, 11, 16, 17, 21, 22, and 23) listed in Table 1.

Also described herein are multispecific binding agents (e.g., antibodies) comprising a second binding domain that binds to PD-L1 and comprises one or more (e.g., one, two or three) VH CDRs and one or more (e.g., one, two or three) VL CDRs listed in Table 2. In particular, described herein is a multispecific binding agent (e.g., an antibody) comprising: a VH CDR1 (SEQ ID NOS: 27, 32, 35, 36, and 40) and a VL CDR1 (SEQ ID NOS: 4, 10, 16, and 21); a VH CDR1 (SEQ ID NOS: 27, 32, 35, 36, and 40) and a VL CDR2 (SEQ ID NOS: 11, 30, and 43); a VH CDR1 (SEQ ID NOS: 27, 32, 35, 36, and 40) and a VL CDR3 (SEQ ID NOS: 31, 39, and 44); a VH CDR2 (SEQ ID NOS: 28, 33, 37, 41, and 45) and a VL CDR1 (SEQ ID NOS: 4, 10, 16, and 21); a VH CDR2 (SEQ ID NOS: 28, 33, 37, 41, and 45) and a VL CDR2 (SEQ ID NOS: 11, 30, and 43); a VH CDR2 (SEQ ID NOS: 28, 33, 37, 41, and 45) and a VL CDR3 (SEQ ID NOS: 31, 39, and 44); a VH CDR3 (SEQ ID NOS: 29, 34, 38, and 42) and a VL CDR1 (SEQ ID NOS: 4, 10, 16, and 21); a VH CDR3 (SEQ ID NOS: 29, 34, 38, and 42) and a VL CDR2 (SEQ ID NOS: 11, 30, and 43); a VH CDR3 (SEQ ID NOS: 29, 34, 38, and 42) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45) and a VL CDR1 (SEQ ID NOS: 4, 10, 16, and 21); a VH CDR1 (SEQ ID NOS: 27, 32, 35, 36, and 40), a VH CDR2 (SEQ ID NOS: 28, 33, 37, 41, and 45) and a VL CDR2 (SEQ ID NOS: 11, 30, and 43); a VH CDR1 (SEQ ID NOS: 27, 32, 35, 36, and 40), a VH CDR2 (SEQ ID NOS: 28, 33, 37, 41, and 45) and a VL CDR3 (SEQ ID NOS: 31, 39, and 44); a VH CDR2 (SEQ ID NOS: 28, 33, 37, 41, and 45), a VH CDR3 (SEQ ID NOS: 29, 34, 38, and 42) and a VL CDR1 (SEQ ID NOS: 4, 10, 16, and 21); a VH CDR2 (SEQ ID NOS: 28, 33, 37, 41, and 45), a VH CDR3 (SEQ ID NOS: 29, 34, 38, and 42) and a VL CDR2 (SEQ ID NOS: 11, 30, and 43); a VH CDR2 (SEQ ID NOS: 28, 33, 37, 41, and 45), a VH CDR3 (SEQ ID NOS: 29, 34, 38, and 42) and a VL CDR3 (SEQ ID NOS: 31, 39, and 44); a VH CDR1 (SEQ ID NOS: 27, 32, 35, 36, and 40), a VL CDR1 (SEQ ID NOS: 4, 10, 16, and 21) and a VL CDR2 (SEQ ID NOS: 11, 30, and 43); a VH CDR1 (SEQ ID NOS: 27, 32, 35, 36, and 40), a VL CDR1 (SEQ ID NOS: 4, 10, 16, and 21) and a VL CDR3 (SEQ ID NOS: 31, 39, and 44); a VH CDR1 (SEQ ID NOS: 27, 32, 35, 36, and 40), a VL CDR2 (SEQ ID NOS: 11, 30, and 43) and a VL CDR3 (SEQ ID NOS: 31, 39, and 44); a VH CDR2 (SEQ ID NOS: 28, 33, 37, 41, and 45), a VL CDR1 (SEQ ID NOS: 4, 10, 16, and 21) and a VL CDR2 (SEQ ID NOS: 11, 30, and 43); a VH CDR2 (SEQ ID NOS: 28, 33, 37, 41, and 45), a VL CDR1 (SEQ ID NOS: 4, 10, 16, and 21) and a VL CDR3 (SEQ ID NOS: 31, 39, and 44); a VH CDR2 (SEQ ID NOS: 28, 33, 37, 41, and 45), a VL CDR2 (SEQ ID NOS: 11, 30, and 43) and a VL CDR3 (SEQ ID NOS: 31, 39, and 44); a VH CDR3 (SEQ ID NOS: 29, 34, 38, and 42), a VL CDR1 (SEQ ID NOS: 4, 10, 16, and 21) and a VL CDR2 (SEQ ID NOS: 11, 30, and 43); a VH CDR3 (SEQ ID NOS: 29, 34, 38, and 42), a VL CDR1 (SEQ ID NOS: 4, 10, 16, and 21) and a VL CDR3 (SEQ ID NOS: 31, 39, and 44); a VH CDR3 (SEQ ID NOS: 29, 34, 38, and 42), a VL CDR2 (SEQ ID NOS: 11, 30, and 43) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42) and a VL CDR1 (SEQ ID NOS: 4, 10, 16, and 21); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42) and a VL CDR2 (SEQ ID NOS: 11, 30, and 43); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VL CDR1 (SEQ ID NOS: 4, 10, 16, and 21) and a VL CDR2 (SEQ ID NOS: 11, 30, and 43); a VH CDR1 (SEQ ID NOS: 27, 32, 35, 36, and 40), a VH CDR2 (SEQ ID NOS: 28, 33, 37, 41, and 45), a VL CDR1 (SEQ ID NOS: 4, 10, 16, and 21) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VL CDR2 (SEQ ID NOS: 11, 30, and 43) and a VL CDR3 (SEQ ID NOS: 31, 39, and 44); a VH CDR1 (SEQ ID NOS: 27, 32, 35, 36, and 40), a VH CDR3 (SEQ ID NOS: 29, 34, 38, and 42), a VL CDR1 (SEQ ID NOS: 4, 10, 16, and 21) and a VL CDR2 (SEQ ID NOS: 11, 30, and 43); a VH CDR1 (SEQ ID NOS: 27, 32, 35, 36, and 40), a VH CDR3 (SEQ ID NOS: 29, 34, 38, and 42), a VL CDR1 (SEQ ID NOS: 4, 10, 16, and 21) and a VL CDR3 (SEQ ID NOS: 31, 39, and 44); a VH CDR1 (SEQ ID NOS: 27, 32, 35, 36, and 40), a VH CDR3 (SEQ ID NOS: 29, 34, 38, and 42), a VL CDR2 (SEQ ID NOS: 11, 30, and 43) and a VL CDR3 (SEQ ID NOS: 31, 39, and 44); a VH CDR2 (SEQ ID NOS: 28, 33, 37, 41, and 45), a VH CDR3 (SEQ ID NOS: 29, 34, 38, and 42), a VL CDR1 (SEQ ID NOS: 4, 10, 16, and 21) and a VL CDR2 (SEQ ID NOS: 11, 30, and 43); a VH CDR2 (SEQ ID NOS: 28, 33, 37, 41, and 45), a VH CDR3 (SEQ ID NOS: 29, 34, 38, and 42), a VL CDR1 (SEQ ID NOS: 4, 10, 16, and 21) and a VL CDR3 (SEQ ID NOS: 31, 39, and 44); a VH CDR2 (SEQ ID NOS: 28, 33, 37, 41, and 45), a VH CDR3 (SEQ ID NOS: 29, 34, 38, and 42), a VL CDR2 (SEQ ID NOS: 11, 30, and 43) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42), a VL CDR1 (SEQ ID NOS: 4, 10, 16, and 21) and a VL CDR2 (SEQ ID NOS: 11, 30, and 43); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42), a VL CDR1 (SEQ ID NOS: 4, 10, 16, and 21) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VH CDR3 (SEQ ID NOS: 29, 34, 38, and 42), a VL CDR2 (SEQ ID NOS: 11, 30, and 43) and a VL CDR3 (SEQ ID NOS: 31, 39, and 44); a VH CDR1 (SEQ ID NOS: 27, 32, 35, 36, and 40), a VH CDR2 (SEQ ID NOS: 28, 33, 37, 41, and 45), a VL CDR1 (SEQ ID NOS: 4, 10, 16, and 21), a VL CDR2 (SEQ ID NOS: 11, 30, and 43), and a VL CDR3 (SEQ ID NOS: 31, 39, and 44); a VH CDR1 (SEQ ID NOS: 27, 32, 35, 36, and 40), a VH CDR3 (SEQ ID NOS: 29, 34, 38, and 42), a VL CDR1 (SEQ ID NOS: 4, 10, 16, and 21), a VL CDR2 (SEQ ID NOS: 11, 30, and 43), and a VL CDR3 (SEQ ID NOS: 31, 39, and 44); a VH CDR2 (SEQ ID NOS: 28, 33, 37, 41, and 45), a VH CDR3 (SEQ ID NOS: 29, 34, 38, and 42), a VL CDR1 (SEQ ID NOS: 4, 10, 16, and 21), a VL CDR2 (SEQ ID NOS: 11, 30, and 43), and a VL CDR3 (SEQ ID NOS: 31, 39, and 44); or any combination thereof of the VH CDRs (SEQ ID NOS: 27, 28, 29, 32, 33, 34, 35, 36, 37, 38, 40, 41, 42, and 45) and VL CDRs (SEQ ID NOS: 4, 30, 31, 10, 11, 16, 39, 21, 43, and 44) listed in Table 2.

In some embodiments, described herein is a multispecific antibody or fragment thereof with a first binding domain that binds to CD47, wherein the first binding domain comprises: (a) a heavy chain variable (VH) region comprising: (1) a VH CDR1 having an amino acid sequence selected from the group consisting of: (i) SEQ ID NO:1, (ii) SEQ ID NO:7, (iii) SEQ ID NO:12, (iv) SEQ ID NO:13, and (v) SEQ ID NO: 18; (2) a VH CDR2 having an amino acid sequence selected from the group consisting of: (i) SEQ ID NO:2, (ii) SEQ ID NO:8, (iii) SEQ ID NO: 14, (iv) SEQ ID NO: 19, and (v) SEQ ID NO:24; and (3) a VH CDR3 having an amino acid sequence selected from the group consisting of: (i) SEQ ID NO:3, (ii) SEQ ID NO:9, (iii) SEQ ID NO: 15, and (iv) SEQ ID NO:20; and (b) a light chain variable (VL) region comprising: (1) a VL CDR1 having an amino acid sequence selected from the group consisting of: (i) SEQ ID NO:4, (ii) SEQ ID NO: 10, (iii) SEQ ID NO: 16, and (iv) SEQ ID NO: 21; (2) a VL CDR2 having an amino acid sequence selected from the group consisting of: (i) SEQ ID NO:5, (ii) SEQ ID NO:11, and (iii) SEQ ID NO:22; and (3) a VL CDR3 having an amino acid sequence selected from the group consisting of: (i) SEQ ID NO:6, (ii) SEQ ID NO:17, and (iii) SEQ ID NO:23. In some embodiments, the multispecific antibody comprises a second binding domain that binds to PD-L1. In some embodiments, the multispecific antibody or fragment is a bispecific antibody. In some embodiments, the second binding domain comprises: (a) a heavy chain variable (VH) region comprising: (1) a VH CDR1 having an amino acid sequence selected from the group consisting of: (i) SEQ ID NO:27, (ii) SEQ ID NO:32, (iii) SEQ ID NO:35, (iv) SEQ ID NO:36, and (v) SEQ ID NO:40; (2) a VH CDR2 having an amino acid sequence selected from the group consisting of: (i) SEQ ID NO:28, (ii) SEQ ID NO:33, (iii) SEQ ID NO:37, (iv) SEQ ID NO:41, and (v) SEQ ID NO:45; and (3) a VH CDR3 having an amino acid sequence selected from the group consisting of: (i) SEQ ID NO:29, (ii) SEQ ID NO:34, (iii) SEQ ID NO:38, (iv) SEQ ID NO:42; and (b) a light chain variable (VL) region comprising: (1) a VL CDR1 having an amino acid sequence selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence selected from the group consisting of: (i) SEQ ID NO:30, (ii) SEQ ID NO:11, and (iii) SEQ ID NO:43; and (3) a VL CDR3 having an amino acid sequence selected from the group consisting of: (i) SEQ ID NO:31, (ii) SEQ ID NO: 39, (iii) SEQ ID NO:44. In some embodiments, described herein is a multispecific antibody or fragment thereof with a first binding domain that binds to CD47, wherein the first binding domain comprises a heavy chain variable (VH) region comprising: (1) a VH CDR1 having an amino acid sequence selected from the group consisting of: (i) SEQ ID NO:1, (ii) SEQ ID NO:7, (iii) SEQ ID NO: 12, (iv) SEQ ID NO:13, and (v) SEQ ID NO: 18; (2) a VH CDR2 having an amino acid sequence selected from the group consisting of: (i) SEQ ID NO:2, (ii) SEQ ID NO:8, (iii) SEQ ID NO:14, (iv) SEQ ID NO:19, and (v) SEQ ID NO:24; and (3) a VH CDR3 having an amino acid sequence selected from the group consisting of: (i) SEQ ID NO: 3, (ii) SEQ ID NO:9, (iii) SEQ ID NO: 15, and (iv) SEQ ID NO:20. In some embodiments, the multispecific antibody comprises a second binding domain that binds to PD-L1. In some embodiments, the multispecific antibody or fragment thereof is a bispecific antibody. In some embodiments, the second binding domain comprises: (a) a heavy chain variable (VH) region comprising: (1) a VH CDR1 having an amino acid sequence selected from the group consisting of: (i) SEQ ID NO: 27, (ii) SEQ ID NO:32, (iii) SEQ ID NO:35, (iv) SEQ ID NO:36, and (v) SEQ ID NO: 40; (2) a VH CDR2 having an amino acid sequence selected from the group consisting of: (i) SEQ ID NO:28, (ii) SEQ ID NO:33, (iii) SEQ ID NO:37, (iv) SEQ ID NO: 41, and (v) SEQ ID NO:45; and (3) a VH CDR3 having an amino acid sequence selected from the group consisting of: (i) SEQ ID NO:29, (ii) SEQ ID NO:34, (iii) SEQ ID NO: 38, and (iv) SEQ ID NO:42.

In some embodiments, described herein is a multispecific antibody or fragment thereof with a first binding domain that binds to CD47, wherein the first binding domain comprises a light chain variable (VL) region comprising: (1) a VL CDR1 having an amino acid sequence selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence selected from the group consisting of: (i) SEQ ID NO:5, (ii) SEQ ID NO:11, and (iii) SEQ ID NO:22; (3) a VL CDR3 having an amino acid sequence selected from the group consisting of: (i) SEQ ID NO:6, (ii) SEQ ID NO:17, and (iii) SEQ ID NO:23. In some embodiments, the multispecific antibody comprises a second binding domain that binds to PD-L1. In some embodiments, the multispecific antibody or fragment thereof is a bispecific antibody. In some embodiments, the second binding domain comprises: (a) a light chain variable (VL) region comprising: (1) a VL CDR1 having an amino acid sequence selected from the group consisting of: (i) SEQ ID NO:4, (ii) SEQ ID NO:10, (iii) SEQ ID NO:16, and (iv) SEQ ID NO:21; (2) a VL CDR2 having an amino acid sequence selected from the group consisting of: (i) SEQ ID NO:30, (ii) SEQ ID NO: 11, and (iii) SEQ ID NO:43; and (3) a VL CDR3 having an amino acid sequence selected from the group consisting of: (i) SEQ ID NO:31, (ii) SEQ ID NO:39, (iii) SEQ ID NO:44.

In some embodiments, described herein is a multispecific antibody or fragment thereof with a first binding domain that binds to CD47, wherein the first binding domain comprises all three heavy chain complementarity determining regions (CDRs) or all three light chain CDRs from: the antibody designated mAb-C that comprises a VH sequence that is SEQ ID NO:25 and a VL sequence that is SEQ ID NO:26. In some embodiments, the multispecific antibody comprises a second binding domain that binds to PD-L1. In some embodiments, the multispecific antibody is a bispecific antibody. In some embodiments, the second binding domain comprises all three heavy chain complementarity determining regions (CDRs) or all three light chain CDRs from: the antibody designated mAb-P that comprises a VH sequence that is SEQ ID NO:46 and a VL sequence that is SEQ ID NO:47.

In some embodiments, described herein is a multispecific antibody or fragment thereof with a first binding domain that binds to CD47, wherein the first binding domain comprises all three heavy chain CDRs and/or all three light chain CDRs from the antibody designated mAb-C. In some embodiments, the multispecific antibody comprises a second binding domain that binds to PD-L1. In some embodiments, the multispecific antibody or fragment thereof is a bispecific antibody. In some embodiments, the second binding domain comprises all three heavy chain CDRs and all three light chain CDRs from the antibody designated mAb-P.

In some embodiments, described herein is a multispecific antibody or fragment thereof with a first binding domain that binds to CD47, wherein the first binding domain comprises: (a) a heavy chain variable (VH) region comprising a VH CDR1, a VH CDR2, and a VH CDR3 amino acid sequence set forth in Table 1; or (b) a light chain variable (VL) region comprising a VL CDR1, a VL CDR2, and a VL CDR3 amino acid sequence set forth in Table 1. In some embodiments, the first binding domain comprises: (a) a heavy chain variable (VH) region comprising a VH CDR1, a VH CDR2, and a VH CDR3 amino acid sequence set forth in Table 1; and (b) a light chain variable (VL) region comprising a VL CDR1, a VL CDR2, and a VL CDR3 amino acid sequence set forth in Table 1. In some embodiments, the multispecific antibody comprises a second binding domain that binds to PD-L1. In some embodiments, the multispecific antibody or fragment thereof is a bispecific antibody. In some embodiments, the second binding domain comprises: (a) a heavy chain variable (VH) region comprising a VH CDR1, a VH CDR2, and a VH CDR3 amino acid sequence set forth in Table 2; and/or (b) a light chain variable (VL) region comprising a VL CDR1, a VL CDR2, and a VL CDR3 amino acid sequence set forth in Table 2. In some embodiments, the first binding domain comprises a heavy chain variable (VH) region comprising a VH CDR1, a VH CDR2, and a VH CDR3 amino acid sequence set forth in Table 1. In some embodiments, the multispecific antibody comprises a second binding domain that binds to PD-L1. In some embodiments, the multispecific antibody or fragment thereof is a bispecific antibody. In some embodiments, the second binding domain comprises a heavy chain variable (VH) region comprising a VH CDR1, a VH CDR2, and a VH CDR3 amino acid sequence set forth in Table 2. In some embodiments, the first binding domain comprises a light chain variable (VL) region comprising a VL CDR1, a VL CDR2, and a VL CDR3 amino acid sequence set forth in Table 1. In some embodiments, the multispecific antibody comprises a second binding domain that binds to PD-L1. In some embodiments, the multispecific antibody or fragment thereof is a bispecific antibody. In some embodiments, the second binding domain comprises a light chain variable (VL) region comprising a VL CDR1, a VL CDR2, and a VL CDR3 amino acid sequence set forth in Table 2.

In some embodiments, described herein is a multispecific antibody or fragment thereof with a first binding domain that binds to CD47, wherein the first binding domain comprises: (a) a heavy chain variable (VH) region comprising: (1) a VH CDR1 having an amino acid sequence selected from the group consisting of SEQ ID NO:1, 7, 12, 13, and 18; (2) a VH CDR2 having an amino acid sequence selected from the group consisting of SEQ ID NO:2, 8, 14, 19 and 24; and (3) a VH CDR3 having an amino acid sequence selected from the group consisting of SEQ ID NO: 3, 9, 15 and 20; and (b) a light chain variable (VL) region comprising: (1) a VL CDR1 having an amino acid sequence selected from the group consisting of SEQ ID NO: 4, 10, 16 and 21; (2) a VL CDR2 having an amino acid sequence selected from the group consisting of SEQ ID NO:5, 11, and 22; and (3) a VL CDR3 having an amino acid sequence selected from the group consisting of SEQ ID NO:6, 17, and 23. In some embodiments, the multispecific antibody comprises a second binding domain that binds to PD-L1. In some embodiments, the first binding domain comprises: (a) a heavy chain variable (VH) region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a heavy chain variable (VH) region comprising: (1) a VH CDR1 having the amino acid sequence of SEQ ID NO:27; (2) a VH CDR2 having the amino acid sequence of SEQ ID NO:28; and (3) a VH CDR3 having the amino acid sequence of SEQ ID NO: 29; and (b) a light chain variable (VL) region comprising: (1) a VL CDR1 having the amino acid sequence of SEQ ID NO:4; (2) a VL CDR2 having the amino acid sequence of SEQ ID NO:30; and (3) a VL CDR3 having the amino acid sequence of SEQ ID NO:31. In some embodiments, wherein the first binding domain comprises: (a) a heavy chain variable (VH) region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a light chain variable (VL) region comprising: (1) a VL CDR1 having the amino acid sequence of SEQ ID NO: 10; (2) a VL CDR2 having the amino acid sequence of SEQ ID NO: 11; and (3) a VL CDR3 having the amino acid sequence of SEQ ID NO:6. In some embodiments, the second binding domain comprises: (a) a heavy chain variable (VH) region comprising: (1) a VH CDR1 having the amino acid sequence of SEQ ID NO:32; (2) a VH CDR2 having the amino acid sequence of SEQ ID NO:33; and (3) a VH CDR3 having the amino acid sequence of SEQ ID NO: 34; and (b) a light chain variable (VL) region comprising: (1) a VL CDR1 having the amino acid sequence of SEQ ID NO: 10; (2) a VL CDR2 having the amino acid sequence of SEQ ID NO: 11; and (3) a VL CDR3 having the amino acid sequence of SEQ ID NO:31. In some embodiments, the first binding domain comprises: (a) a heavy chain variable (VH) region comprising: (1) a VH CDR1 having the amino acid sequence of SEQ ID NO: 12; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a heavy chain variable (VH) region comprising: (1) a VH CDR1 having the amino acid sequence of SEQ ID NO:35; (2) a VH CDR2 having the amino acid sequence of SEQ ID NO:28; and (3) a VH CDR3 having the amino acid sequence of SEQ ID NO: 29; and (b) a light chain variable (VL) region comprising: (1) a VL CDR1 having the amino acid sequence of SEQ ID NO:4; (2) a VL CDR2 having the amino acid sequence of SEQ ID NO:30; and (3) a VL CDR3 having the amino acid sequence of SEQ ID NO:31. In some embodiments, first binding domain comprises: (a) a heavy chain variable (VH) region comprising: (1) a VH CDR1 having the amino acid sequence of SEQ ID NO:13; (2) a VH CDR2 having the amino acid sequence of SEQ ID NO: 14; and (3) a VH CDR3 having the amino acid sequence of SEQ ID NO: 15; and (b) a light chain variable (VL) region comprising: (1) a VL CDR1 having the amino acid sequence of SEQ ID NO: 16; (2) a VL CDR2 having the amino acid sequence of SEQ ID NO: 11; and (3) a VL CDR3 having the amino acid sequence of SEQ ID NO:17. In some embodiments, the second binding domain comprises: (a) a heavy chain variable (VH) region comprising: (1) a VH CDR1 having the amino acid sequence of SEQ ID NO:36; (2) a VH CDR2 having the amino acid sequence of SEQ ID NO:37; and (3) a VH CDR3 having the amino acid sequence of SEQ ID NO: 38; and (b) a light chain variable (VL) region comprising: (1) a VL CDR1 having the amino acid sequence of SEQ ID NO: 16; (2) a VL CDR2 having the amino acid sequence of SEQ ID NO: 11; and (3) a VL CDR3 having the amino acid sequence of SEQ ID NO:39. In some embodiments, first binding domain comprises: (a) a heavy chain variable (VH) region comprising: (1) a VH CDR1 having the amino acid sequence of SEQ ID NO:18; (2) a VH CDR2 having the amino acid sequence of SEQ ID NO: 19; and (3) a VH CDR3 having the amino acid sequence of SEQ ID NO: 20; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:23. In some embodiments, the second binding domain comprises: (a) a heavy chain variable (VH) region comprising: (1) a VH CDR1 having the amino acid sequence of SEQ ID NO:40; (2) a VH CDR2 having the amino acid sequence of SEQ ID NO:41; and (3) a VH CDR3 having the amino acid sequence of SEQ ID NO: 42; and (b) a light chain variable (VL) region comprising: (1) a VL CDR1 having the amino acid sequence of SEQ ID NO:21; (2) a VL CDR2 having the amino acid sequence of SEQ ID NO:43; and (3) a VL CDR3 having the amino acid sequence of SEQ ID NO:44. In some embodiments, the first binding domain comprises: (a) a heavy chain variable (VH) region comprising: (1) a VH CDR1 having the amino acid sequence of SEQ ID NO:1; (2) a VH CDR2 having the amino acid sequence of SEQ ID NO: 24; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6. In some embodiments, the second binding domain comprises: (a) a heavy chain variable (VH) region comprising: (1) a VH CDR1 having the amino acid sequence of SEQ ID NO:27; (2) a VH CDR2 having the amino acid sequence of SEQ ID NO:45; and (3) a VH CDR3 having the amino acid sequence of SEQ ID NO: 29; and (b) a light chain variable (VL) region comprising: (1) a VL CDR1 having the amino acid sequence of SEQ ID NO:4; (2) a VL CDR2 having the amino acid sequence of SEQ ID NO:30; and (3) a VL CDR3 having the amino acid sequence of SEQ ID NO:31. In some embodiments, a multispecific antibody or fragment thereof described above is a bispecific antibody.

In some embodiments, described herein is a binding agent that binds to essentially the same epitope as an antibody or fragment thereof of any one of the antibodies described herein. In some embodiments, described herein is a binding agent that competes for binding to PD-L1 (such as human PD-L1) with an antibody or fragment thereof of any one described herein. Additionally or alternatively, described herein is a binding agent that competes for binding to CD47 (such as human CD47) with an antibody or fragment thereof of any one described herein. In some embodiments, the binding agent is an antibody or fragment thereof.

In certain aspects, the CDRs of a multispecific binding agent (e.g., an antibody, such as a bispecific antibody), including a multispecific binding agent that binds to CD47, including human CD47, and one or more targets that are not CD47 (e.g., PD-L1, including human PD-L1), can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain aspects, the CDRs of a multispecific binding agent (e.g., an antibody, such as a bispecific antibody), including a multispecific binding agent that binds to CD47, including human CD47, and one or more targets that are not CD47 (e.g., PD-L1, including human PD-L1), can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226).

In certain aspects, the CDRs of a multispecific binding agent (e.g., an antibody, such as a bispecific antibody), including a multispecific binding agent that binds to CD47, including human CD47, and one or more targets that are not CD47 (e.g., PD-L1, including human PD-L1), can be determined according to the ImMunoGeneTics (IMGT) system, for example, as described in Lefranc, M.-P., 1999, The Immunologist, 7:132-136 and Lefranc, M.-P. et al., 1999, Nucleic Acids Res., 27:209-212 (“IMGT CDRs”).

In certain aspects, the CDRs of a multispecific binding agent (e.g., an antibody, such as a bispecific antibody), including a multispecific binding agent that binds to CD47, including human CD47, and one or more targets that are not CD47 (e.g., PD-L1, including human PD-L1), can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of a multispecific binding agent (e.g., an antibody, such as a bispecific antibody), including a multispecific binding agent that binds to CD47, including human CD47, and one or more targets that are not CD47 (e.g., PD-L1, including human PD-L1), can be determined according to the Contact system, which will be referred to herein as the “Contact CDRs” (see, e.g., MacCallum R M et al., 1996, J Mol Biol 5:732-745). The Contact CDRs are based on an analysis of the available complex crystal structures.

In some embodiments, the position of one or more CDRs along the VH (e.g., CDR1, CDR2, or CDR3) and/or VL (e.g., CDR1, CDR2, or CDR3) region of a first binding domain of a multispecific binding agent (e.g., an antibody, such as a bispecific antibody), including a multispecific binding agent that binds to CD47, including human CD47, and one or more targets that are not CD47 (e.g., PD-L1, including human PD-L1), described herein may vary by one, two, three, four, five, or six amino acid positions so long as binding to CD47 (e.g., human CD47) is maintained (e.g., substantially maintained, for example, at least 50%, at least 60%, at least 70%, at least 80%, at least 90%, or at least 95%). For example, in some embodiments, the position defining a CDR of Table 1 may vary by shifting the N-terminal and/or C-terminal boundary of the CDR by one, two, three, four, five, or six amino acids, relative to the current CDR position, so long as binding to CD47 (e.g., human CD47) is maintained (e.g., substantially maintained, for example, at least 50%, at least 60%, at least 70%, at least 80%, at least 90%, or at least 95%). Additionally or alternatively, the length of one or more CDRs along the VH (e.g., CDR1, CDR2, or CDR3) and/or VL (e.g., CDR1, CDR2, or CDR3) region of a first binding domain of a multispecific binding agent (e.g., an antibody, such as a bispecific antibody), including a multispecific binding agent that binds to CD47, including human CD47, and one or more targets that are not CD47 (e.g., PD-L1, including human PD-L1), described herein may vary (e.g., be shorter or longer) by one, two, three, four, five, or more amino acids, so long as binding to CD47 (e.g., human CD47) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 of a first binding domain described herein may be extended by one, two, three, four, five or more amino acids compared to one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). Additionally or alternatively, the carboxy terminus of a VH and/or VL CDR1, CDR2, and/or CDR3 of a first binding domain described herein may be extended or shortened by one, two, three, four, five or more amino acids compared to one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). In other embodiments, the amino terminus of a VH and/or VL CDR1, CDR2, and/or CDR3 of a first binding domain described herein may be shortened by one, two, three, four, five or more amino acids compared to one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). Additionally or alternatively, the carboxy terminus of a VH and/or VL CDR1, CDR2, and/or CDR3 of a first binding domain described herein may be extended or shortened by one, two, three, four, five or more amino acids compared to one or more of the CDRs described by SEQ ID NOS: 1-24, so long as binding to CD47 (e.g., human CD47) is maintained (e.g., substantially maintained, for example, at least 50%, at least 60%, at least 70%, at least 80%, at least 90%, or at least 95%). Any method known in the art can be used to ascertain whether binding to CD47 (e.g., human CD47) is maintained, for example, the binding assays and conditions described in the “Examples” section described herein.

In some embodiments, the position of one or more CDRs along the VH (e.g., CDR1, CDR2, or CDR3) and/or VL (e.g., CDR1, CDR2, or CDR3) region of a second binding domain of a multispecific binding agent (e.g., an antibody, such as a bispecific antibody), including a multispecific binding agent that binds to CD47, including human CD47, and PD-L1, including human PD-L1, described herein may vary by one, two, three, four, five, or six amino acid positions so long as binding to PD-L1 (e.g., human PD-L1) is maintained (e.g., substantially maintained, for example, at least 50%, at least 60%, at least 70%, at least 80%, at least 90%, or at least 95%). For example, in some embodiments, the position defining a CDR of Table 2 may vary by shifting the N-terminal and/or C-terminal boundary of the CDR by one, two, three, four, five, or six amino acids, relative to the current CDR position, so long as binding to PD-L1 (e.g., human PD-L1) is maintained (e.g., substantially maintained, for example, at least 50%, at least 60%, at least 70%, at least 80%, at least 90%, or at least 95%). Additionally or alternatively, the length of one or more CDRs along the VH (e.g., CDR1, CDR2, or CDR3) and/or VL (e.g., CDR1, CDR2, or CDR3) region of a second binding domain of a multispecific binding agent (e.g., an antibody, such as a bispecific antibody), including a multispecific binding agent that binds to CD47, including human CD47, and PD-L1, including human PD-L1, described herein may vary (e.g., be shorter or longer) by one, two, three, four, five, or more amino acids, so long as binding to PD-L1 (e.g., human PD-L1) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 of a second binding domain described herein may be extended by one, two, three, four, five or more amino acids compared to one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). Additionally or alternatively, the carboxy terminus of a VH and/or VL CDR1, CDR2, and/or CDR3 of a second binding domain described herein may be extended or shortened by one, two, three, four, five or more amino acids compared to one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). In other embodiments, the amino terminus of a VH and/or VL CDR1, CDR2, and/or CDR3 of a second binding domain described herein may be shortened by one, two, three, four, five or more amino acids compared to one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). Additionally or alternatively, the carboxy terminus of a VH and/or VL CDR1, CDR2, and/or CDR3 of a second binding domain described herein may be shortened by one, two, three, four, five or more amino acids compared to one or more of the CDRs described by SEQ ID NOS: 27-45, so long as binding to PD-L1 (e.g., human PD-L1) is maintained (e.g., substantially maintained, for example, at least 50%, at least 60%, at least 70%, at least 80%, at least 90%, or at least 95%). Any method known in the art can be used to ascertain whether binding to PD-L1 (e.g., human PD-L1) is maintained, for example, the binding assays and conditions described in the “Examples” section described herein.

In some embodiments, described herein is a multispecific antibody comprising a VH region and/or VL region described herein, which further comprises human framework sequences. In some embodiment, the VH region and/or VL region further comprises a framework 1 (FR1), a framework 2 (FR2), a framework 3 (FR3) and/or a framework 4 (FR4) sequence.

In some embodiments, the multispecific binding agents (e.g., antibodies, such as bispecific antibodies) that bind to CD47, including multispecific binding agents that bind to CD47, including human CD47, and one or more targets that are not CD47 (e.g., PD-L1, including human PD-L1), presented herein, comprise one or more conservative sequence modifications. With respect to polypeptides that are multispecific binding agents (e.g., antibodies, such as bispecific antibodies), such as multispecific binding agents that bind to CD47, including human CD47, and one or more targets that are not CD47 (e.g., PD-L1, including human PD-L1), conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, a predicted nonessential amino acid residue in a binding agent as described herein is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)). In some embodiments, the conservative sequence modifications described herein modify the amino acid sequences of the multispecific binding agents (e.g., antibodies, such as bispecific antibodies), including multispecific binding agents that bind to CD47, including human CD47, and one or more targets that are not CD47 (e.g., PD-L1, including human PD-L1), by 50%, or 55%, or 60%, or 65%, or 70%, or 75%, or 80%, or 85%, or 90%, or 95%, or 98%, or 99%. In some embodiments, the nucleotide and amino acid sequence modifications refer to at most 1, 2, 3, 4, 5, or 6 amino acid substitutions to the CDRs described in Table 1 or Table 2. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, a binding agent as described herein comprises a conservative amino acid substitution outside of any CDRs. In further embodiments, a binding agent as described herein comprises a conservative amino acid substitution in an FR. Additionally or alternatively, a binding agent as described herein comprises a conservative amino acid substitution in a constant region or a constant domain, such as CI, CH1, CH2, or CH3.

The present disclosure provides humanized antibodies that bind CD47, including human CD47, and one or more targets that are not CD47 (e.g, PD-L1, including human PD-L1). Humanized antibodies of the present disclosure may comprise a first binding domain that binds to CD47 and comprises one or more CDRs as shown in Table 1. Humanized antibodies of the present disclosure may comprise a second binding domain that binds to PD-L1 and comprises one or more CDRs as shown in Table 2. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind CD47 may be produced using techniques known to those skilled in the art (Zhang et al., Molecular Immunology, 42 (12): 1445-1451, 2005; Hwang et al., Methods, 36 (1): 35-42, 2005; Dall'Acqua et al., Methods, 36 (1): 43-60, 2005; Clark, Immunology Today, 21 (8): 397-402, 2000, and U.S. Pat. Nos. 6,180,370; 6,054,927; 5,869,619; 5,861,155; 5,712,120; and 4,816,567, all of which are all hereby expressly incorporated herein by reference).

In some cases, the humanized antibodies are constructed by CDR grafting, in which the amino acid sequences of the six complementarity determining regions (CDRs) of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36:25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623. In some cases, the framework is derived from the consensus sequences of the most abundant human subclasses, V_{L}6 subgroup I (V_{L}61) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-824, 2000), Modeller (Sali and Blundell, J. Mol. Biol. 234:779-815, 1993), and Swiss PDB Viewer (Guex and Peitsch, Electrophoresis 18:2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (Lazar et al., Mol. Immunol. 44:1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23:1105-1116, 2005; Dufner et al., Trends Biotechnol. 24:523-529, 2006; Feldhaus et al., Nat. Biotechnol. 21:163-70, 2003; Schlapschy et al., Protein Eng. Des. Sel. 17:847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (see, e.g., Foote and Winter, J. Mol. Biol. 224:487-499, 1992), or from the more limited set of target residues identified by Baca et al. (J. Biol. Chem. 272:10678-10684, 1997).

In FR shuffling, whole FRs are combined with the non-human CDRs instead of creating combinatorial libraries of selected residue variants (see, e.g., Dall'Acqua et al., Methods 36:43-60, 2005). The libraries may be screened for binding in a two-step selection process, first humanizing VL, followed by VH. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44:3049-60, 2007).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7:805-814 (1994), U.S. Pat. Nos. 5,766,886, 5,770,196, 5,821,123, and 5,869,619, and PCT Application Publication WO 93/11794.

“Humanized antibodies” are antibodies in which CDRs of heavy and light variable chains of non-human immunoglobulins are transferred into a human variable domain. Constant regions need not be present, but if they are, they optionally are substantially identical to human immunoglobulin constant regions, e.g., at least about 85-90%, about 95%, 96%, 97%, 98%, 99% or more identical, in some embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

A multispecific binding agent (e.g., an antibody), including a multispecific binding agent that binds to CD47, including human CD47, and one or more targets that are not CD47 (e.g., PD-L1, including human PD-L1), can be genetically engineered. For example, a multispecific binding agent (e.g., an antibody), including a multispecific binding agent that binds to CD47, including human CD47, and one or more targets that are not CD47 (e.g., PD-L1, including human PD-L1) comprises, for example, a variable region domain generated by recombinant DNA engineering techniques. In this regard, a variable region is optionally modified by insertions, deletions, or changes in the amino acid sequence of the antibody to produce an antibody of interest, including as described above. Polynucleotides encoding complementarity determining regions (CDRs) of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2:106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

In certain embodiments, the multispecific binding agent (e.g., a bispecific antibody) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Tables 1-2. In some embodiments, the multispecific binding agent (e.g., a bispecific antibody) provided herein comprises CDRs of any antibody or fragment thereof provided herein, for example in Tables 1-2. In further embodiments, the bispecific antibody provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a VH or VL in Tables 1-2.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecule described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). In some embodiments, the percent identity between two sequences is calculated by dividing the number of residue(s) varied (excluding or including conservative amino acid substitution(s) or degenerate nucleotide substitution(s)) between the two sequences in the alignment with the residue number of any one of the following: (i) full length of the shorter sequence, (ii) full length of the longer sequence, (iii) mean length of the two sequences, (iv) total length of the non-gap portion of the alignment, (v) length of the alignment excluding overhangs, or (vi) length of the alignment including overhangs. Overhangs as used herein with respect to a sequence alignment refer to either or both ends of the alignment where residues of one sequence are considered as aligning to no residues (e.g., gap) in the other sequence. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:25, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:26, and the binding of the multispecific binding agent to CD47 (e.g., human CD47) is maintained (e.g., substantially maintained, for example, at least 50%, at least 60%, at least 70%, at least 80%, at least 90%, or at least 95%).

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:46, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:47, and the binding of the multispecific binding agent to PD-L1 (e.g., human PD-L1) is maintained (e.g., substantially maintained, for example, at least 50%, at least 60%, at least 70%, at least 80%, at least 90%, or at least 95%).

In some embodiments, functional epitopes can be mapped, e.g., by combinatorial alanine scanning, to identify amino acids in the CD47 (or PD-L1) protein that are necessary for interaction with a multispecific binding agent, a CD47 (or PD-L1) binding domain thereof, and/or an anti-CD47 (or anti-PD-L1) antibody provided herein. In some embodiments, conformational and crystal structure of a multispecific binding agent, a CD47 (or PD-L1) binding domain thereof, and/or an anti-CD47 (or anti-PD-L1) antibody bound to a CD47 (or PD-L1) may be employed to identify the epitopes. In some embodiments, the present disclosure provides a multispecific antibody comprising a CD47 (or PD-L1) binding domain that specifically binds to the same epitope as any of the multispecific binding agents as disclosed herein, CD47 (or PD-L1) binding domains thereof, and/or the anti-CD47 (or anti-PD-L1) antibodies or fragments thereof provided herein.

### Binding Agents—Scaffold and Generation

In some embodiments, multispecific binding agents (e.g., antibodies, such as bispecific antibodies) described herein have a combination of (i) a VH domain or VH region; and (ii) a VL domain or VL region. For example, an exemplary bispecific IgG antibody comprises (i) a heavy chain having a combination of a VH domain or VH region as described herein; and one or more heavy chain constant domains or constant regions (e.g., CH1, Hinge, CH2, and CH3), and (ii) a light chain having a combination of a VL domain or VL region as described herein and a light chain constant domain or constant region (CL). An exemplary IgG heavy chain comprises any VH domain as described herein and the following CH1, Hinge, CH2, and CH3 amino acid sequence:
ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKG QPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 65). Another exemplary IgG heavy chain comprises any VH domain as described herein and the following CH1, Hinge, CH2, and CH3 amino acid sequence:

(SEQ ID NO: 66)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALKAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

An exemplary light chain (e.g., for pairing with an IgG heavy chain) comprises any VL domain as described herein and the following CL amino acid sequence:

(SEQ ID NO: 67)
RTVAAPSVFIFPPSDSQLKSGTASVVCLLNNFYPREAKVQWKVDN
ALQSGNSQESVTEQDSKDSTYSLSSTLTLSKADYEKHKVYACEVT
HQGLSSPVTKSFNRGEC.

In some embodiments, mutations are introduced to one or more of the heavy chain constant regions (such as CH1, CH2, and/or CH3) and/or one or more light chain constant regions (such as CL), in order to achieve one or more of the following: (i) destabilizing a homodimer formed by a polypeptide of the multispecific antibody; (ii) stabilizing a multispecific antibody (also referred to herein as a heterodimer) as described herein; (iii) facilitating a proper assembly of a multispecific antibody as described herein; (iv) favoring heterodimerization over homodimerization of the constituent polypeptide chains; (v) improving the yield of a multispecific antibody as described herein; and (vi) improving the purity of a multispecific antibody as described herein. A variety of mutations have been developed that drive preferential heterodimerization, such as knob-in-hole (KIH or KiH) mutations (see, e.g., U.S. Pat. Nos. 5,731,168; 5,807,706; 5,821,333; and 8,216,805), disulfide-stabilized KIH mutations (see, e.g., U.S. Pat. Nos. 7,951,917; 8,642,745; and 9,409,989), and others (see, e.g., WO 2022/125986). Each of the patents and/or patent publications cited herein is incorporated herein by reference in its entirety.

In some specific embodiments, the multispecific antibody provided herein comprises four polypeptides: a first polypeptide comprising from N-terminus to C-terminus: a first VL, a first CH3, an optional hinge, a first CH2, and a second CH3; a second polypeptide comprising from N-terminus to C-terminus: a first VH and a third CH3; a third polypeptide comprising from N-terminus to C-terminus: a second VL, a CL (optionally a kappa CL, also referred to herein as CK), an optional hinge, a second CH2, and fourth CH3; and a fourth polypeptide comprising from N-terminus to C-terminus: a second VH and a CH1. These four polypeptides form two binding domains. In some embodiments, the first polypeptide and the second polypeptide (such as, the first VL and the first VH thereof) form a binding domain that binds to PD-L1, and the third polypeptide and the fourth polypeptide (such as, the second VL and the second VH thereof) form a binding domain that binds to CD47. In other embodiments, the first polypeptide and the second polypeptide (such as, the first VL and the first VH thereof) form a binding domain that binds to CD47, and the third polypeptide and the fourth polypeptide (such as, the second VL and the second VH thereof) form a binding domain that binds to PD-L1. In some embodiments, amino acid sequences of the first CH3, the second CH3, the third CH3 and the fourth CH3, or any subgroup thereof, are identical to each other. In some embodiments, amino acid sequences of the first CH3, the second CH3, the third CH3 and the fourth CH3, or any subgroup thereof, are different from each other. In some embodiments, the second CH3 and the fourth CH3 provide a knobs-in-holes assembly. Additionally or alternatively, amino acid sequences of the first CH2 and the second CH2 are identical to each other. In other embodiments, amino acid sequences of the first CH2 and the second CH2 are different from each other.

In some embodiments, any one or more of the CH3 sequences comprise

GQPREPQVYTLPPSRDELTKNQVSLTCLVKGFYPSDIA
VEWESNGQPENNYKTTPPVLDSDGSFFLYSKLTVDKSR
WQQGNVFSCSVMHEALHNHYTQKSLSLSPGK
(SEQ ID NO: 70; aa 116 to
aa 222 of SEQ ID NO: 62).

In some embodiments, isoallotype mutations D356E and L358M are made in a CH3 sequence as disclosed herein. In some embodiments, one or more isoallotype mutations (for example, either or both D356E and L358M) are in one or more of the CH3 sequences immediately adjacent to the C terminus of a CH2 sequence. Additionally or alternatively, any one or more of the CH3 sequences are engineered to reduce the risk of immunogenicity of the antibody by replacing specific amino acids of one allotype with those of another allotype and referred to herein as isoallotype mutations, as described in more detail in Stickler et al. (Genes Immun. 2011 April; 12 (3): 213-221).

Additionally or alternatively, the CH3 sequences are engineered to comprise knobs-into-holes mutations. In some embodiments, in a CH3/CH3 pair (e.g., one CH3 in one polypeptide is dimerized to another CH3 in a different polypeptide when forming a binding agent comprising the polypeptides) immediately adjacent to the C terminus of a CH2 sequence, one CH3 comprises a knob mutation (for example, T366W) while the other CH3 comprises a hole mutation (for example, any one or any two or all three of T366S, L368A, and Y407V). In other embodiments, one CH3 immediately adjacent to the C terminus of a VH or a VL in a VH/VL pair (e.g., the VH and the VL form a binding domain in a binding agent comprising the VH and VL) comprises a knob mutation (for example, T366W) while the other CH3 (immediately adjacent to the C terminus of a VL or a VH of the same VH/VL pair) comprises hole mutations (for example, any one or any two or all three of T366S, L368A, and Y407V).

As it would be understood by one of skill in the art, a domain pair, such as a CH3/CH3 pair, a VH/VL pair, a VL/VH pair, a CH2/CH2 pair, a CH1/CL pair, or a CL/CH1 pair, refers to one antibody domain (such as VH, VL, CH1, CH2, CH3, or CL) in one polypeptide chain dimerized to another antibody domain (such as VL, VH, CL, CH2, CH3, or CH, respectively) in a different polypeptide chain when forming a binding agent comprising the polypeptides.

In some embodiments, in a CH3/CH3 pair (e.g., one CH3 in one polypeptide is dimerized to another CH3 in a different polypeptide when forming a binding agent comprising the polypeptides), one CH3 comprises a Y349C mutation, while the other CH3 comprises an S354C mutation. In some embodiments, one CH3 immediately adjacent to the C terminus of a VH or a VL in a VH/VL pair comprises an S354C mutation, while the other CH3 (immediately adjacent to the C terminus of a VL or a VH of the same VH/VL pair) comprises a Y349C mutation. Additionally or alternatively, the CH3 sequences are engineered so that they can form a disulfide bond in an antibody, for example in order to stabilize the knobs-into-holes mutations as described above.

Additionally or alternatively, any one or more of the CH3 sequences are engineered to comprise other mutation(s) with the proviso that the mutation(s) do not significantly reduce the affinity and/or stability of the antibody, nor significantly increase the risk of immunogenicity of the antibody. In some embodiments, any one or more of the CH3 sequences are engineered to comprise a mutation as described in WO 2022/125986.

In some embodiments, in a CH3/CH3 pair, one CH3 comprises a mutation of S354C and the other CH3 comprises a mutation of Y349C. Additionally or alternatively, in a CH3/CH3 pair, E357 of one CH3 is substituted with a hydrophobic or aromatic amino acid. In various embodiments, the hydrophobic amino acid residue is selected from the group consisting of: isoleucine (I), leucine (L), methionine (M), proline (P) and valine (V). In various embodiments, the aromatic amino acid is selected from the group consisting of: histidine (H), tryptophan (W), phenylalanine (F), and tyrosine (Y). In some embodiments, the E357 of the CH3 is substituted with W. Additionally or alternatively, in some embodiments, a CH (for example a CH3) comprises a K370R mutation dimerized to an E357-mutation containing CH3 in a binding agent. In some embodiments, one CH3 of a CH3/CH3 pair comprises a K370R mutation. Additionally or alternatively, the other CH3 of the same CH3/CH3 pair comprises a E357W mutation. In some embodiments, one CH3 of a CH3/CH3 pair comprises a K370R mutation, while the other CH3 of the same CH3/CH3 pair comprises a E357W mutation. In some embodiments, in a CH3/CH3 pair, one CH3 comprises S354C and E357W, while the other CH3 comprises Y349C and K370R. In some embodiments, each CH3 of the CH3/CH3 pair is immediately adjacent to the C terminus of a CH2 sequence. In other embodiments, one CH3 of the CH3/CH3 pair is immediately adjacent to the C terminus of a VH or a VL in a VH/VL pair, and the other CH3 of the CH3/CH3 pair is immediately adjacent to the C terminus of a VL or a VH of the same VH/VL pair. In some embodiments, one CH3 immediately adjacent to the C terminus of a VH or a VL in a VH/VL pair comprises E357W; while the other CH3 (immediately adjacent to the C terminus of a VL or a VH of the same VH/VL pair) comprises K370R. In other embodiments, in a CH3/CH3 pair immediately adjacent to the C terminus of a CH2 sequence, one CH3 comprises both S354C and E357W, while the other CH3 comprises both Y349C and K370R.

Additionally or alternatively, one or more amino acid residues in a CH3 are swapped with the corresponding one or more amino acid residues in a CH1. In some embodiments, as used herein, a first amino acid residue in a first peptide corresponding to a second amino acid residue in a second peptide refers to the first amino acid residue aligned to the second amino acid residue in a sequence alignment between the first peptide and the second peptide. Alignment methods are available to one of skilled in the art, such as BLAST as disclosed herein and/or Clustal Omega. In some embodiments, the CH3 is immediately adjacent to C terminus of a VH or VL. Additionally or alternatively, the one or more amino acid residues are in an N-terminus fragment of a CH1, for example selected from the 1^{st} to the 10^{th} (including each range or integer therebetween, such as the 1^{st} to the 5^{th}, the 1^{st} to the 3^{rd}, the 1^{st} or the 3^{rd}) amino acids of a CH1. In some embodiments, a CH3 comprises the first amino acid residue swapped with the first amino acid residue of a CH1, which is also referred to herein as an N-terminal amino acid residue swapped with CH1. In some embodiments, the first amino acid residue of a CH3, which is a G, is substituted with the first amino acid residue of a CH1, which is an A. Such substitution is also designated as G341A herein for the ease of reference. In some embodiments, a CH3 immediately adjacent to C terminus of a VH or VL comprises G341A. Without wishing to be bound by the theory, a CH3 immediately adjacent to C terminus of a VH or VL and engineered to comprise a CH1 N-terminal fragment can improve the assembly and/or the purity of the binding agent as disclosed herein. In some embodiments, in a CH3/CH3 pair, each CH3 of which is immediately adjacent to the C terminus of a VH or a VL, one CH3 comprises the mutations of S354C and E357W, and the other CH3 comprises the mutations of Y349C and K370R. In some embodiments, in a CH3/CH3 pair, each CH3 of which is immediately adjacent to the C terminus of a VH or a VL, one CH3 comprises the mutations of G341A, S354C and E357W, and the other CH3 comprises the mutations of Y349C and K370R. In some embodiments, in a CH3/CH3 pair, each CH3 of which is immediately adjacent to the C terminus of a VH or a VL, one CH3 comprises the mutations of G341A, S354C and E357W, and the other CH3 comprises the mutations of G341A, Y349C and K370R. In some embodiments, in a CH3/CH3 pair, each CH3 of which is immediately adjacent to the C terminus of a VH or a VL, one CH3 comprises the mutations of S354C and E357W, and the other CH3 comprises the mutations of G341A, Y349C and K370R.

In some embodiments, the multispecific binding agent as described herein comprises one or more CH3 mutations as disclosed in WO 2022/125986, which is incorporated herein by reference in its entirety. In some embodiments, the multispecific binding agent as described herein comprises one or more CH3 domains as disclosed in WO 2022/125986.

Accordingly, any one or more of the CH3 sequences comprise any one of the following:

GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
(SEQ ID NO: 70; aa 116 to aa 222
of SEQ ID NO: 62);
GQPREPQVCTLPPSRDELTKNQVSLTCLVRGFYPSDIAVEWESNG
QPENNYKTTPPVLDSDGSFFLYSKLTVDKSRWQQGNVFSCSVMHE
ALHNHYTQKSLSLSPGK
(SEQ ID NO: 71; Y349C and K370R);
AQPREPQVCTLPPSRDELTKNQVSLTCLVRGFYPSDIAVEWESNG
QPENNYKTTPPVLDSDGSFFLYSKLTVDKSRWQQGNVFSCSVMHE
ALHNHYTQKSLSLSPGK
(SEQ ID NO: 72; N-terminal amino acid residue
swapped with CH1, Y349C and K370R);
GQPREPQVYTLPPCRDWLTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
(SEQ ID NO: 73; S354C and E357W);
AQPREPQVYTLPPCRDWLTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
(SEQ ID NO: 74; N-terminal amino acid residue
swapped with CH1, S354C and E357W);
GQPREPQVYTLPPSREEMTKNQVSLSCAVKGFYPSDIAVEWESNG
QPENNYKTTPPVLDSDGSFFLVSKLTVDKSRWQQGNVFSCSVMHE
ALHNHYTQKSLSLSPGK
(SEQ ID NO: 75; D356E, L358M, T366S, L368A
and Y407V);
or
GQPREPQVYTLPPSRDELTKNQVSLWCLVKGFYPSDIAVEWESNG
QPENNYKTTPPVLDSDGSFFLYSKLTVDKSRWQQGNVFSCSVMHE
ALHNHYTQKSLSLSPGK
(SEQ ID NO: 76; T366W).

In some embodiments, a binding agent as disclosed herein comprises a CH3 sequence as disclosed herein (such as any one of SEQ ID Nos: 70-74) but the CH3 lacks the C-terminal amino acid residue of lysine (K). In further embodiments, the CH3 is at the C terminus of any one or more polypeptides of the binding agent. In some embodiments, a C-terminus lysine was cleaved off in one or more polypeptides of the binding agent, such as any one or any two or any three of the following: the first polypeptide of a binding agent as disclosed herein, the second polypeptide of a binding agent as disclosed herein, and the third polypeptide of a binding agent as disclosed herein.

In some embodiments, one CH3 immediately adjacent to the C terminus of a VH or a VL in a VH/VL pair comprises GQPREPQVCTLPPSRDELTKNQVSLTCLVRGFYPSDIAVEWESNGQPENNYKTTP PVLDSDGSFFLYSKLTVDKSRWQQGNVFSCSVMHEALHNHYTQKSLSLSPGK (SEQ ID NO:71; Y349C and K370R); while the other CH3 (immediately adjacent to the C terminus of a VL or a VH of the same VH/VL pair) comprises AQPREPQVYTLPPCRDWLTKNQVSLTCLVKGFYPSDIAVEWESNGQPENNYKTTP PVLDSDGSFFLYSKLTVDKSRWQQGNVFSCSVMHEALHNHYTQKSLSLSPGK (SEQ ID NO:74; N-terminal amino acid residue swapped with CH1, S354C and E357W).

In some embodiments, in a CH3/CH3 pair immediately adjacent to the C terminus of a CH2 sequence, one CH3 comprises GQPREPQVYTLPPSREEMTKNQVSLSCAVKGFYPSDIAVEWESNGQPENNYKTTP PVLDSDGSFFLVSKLTVDKSRWQQGNVFSCSVMHEALHNHYTQKSLSLSPGK (SEQ ID NO:75; D356E, L358M, T366S, L368A and Y407V), while the other CH3 comprises

(SEQ ID NO: 76; T366W)
GQPREPQVYTLPPSRDELTKNQVSLWCLVKGFYPSDIA
VEWESNGQPENNYKTTPPVLDSDGSFFLYSKLTVDKSR
WQQGNVFSCSVMHEALHNHYTQKSLSLSPGK.

As it will be understood by one of skill in the art and if not specified otherwise, EU numbering (also referred to herein as EU indexing) is referred to herein when describing a mutation in an antibody or a fragment thereof, such as an Fc or a CH3. See, more details at www.imgt.org/IMGTScientificChart/Numbering/Hu_IGHGnber.html #refs, which is hereby incorporated by reference in its entirety and identifies the residue according to its location in an endogenous constant region sequence regardless of the residue's physical location within a chain of the antibody constructs described herein. For example, in a CH3 consisting of aa 116 to aa 222 of SEQ ID NO: 62, the 1^{st} aa of the CH3 (e.g., the 116^{th} aa of SEQ ID NO: 62) is numbered as 341 and is referred to herein as G341; the 9^{th} aa of the CH3 is referred to herein as Y349; the 14^{th} aa of the CH3 is referred to herein as S354; the 16^{th} aa of the CH3 is referred to herein as D356; the 17^{th} aa of the CH3 is referred to herein as E357; the 18^{th} aa of the CH3 is referred to herein as L358; the 26^{th} aa of the CH3 is referred to herein as T366; the 28^{th} aa of the CH3 is referred to herein as L368; the 30^{th} aa of the CH3 is referred to herein as K370; and the 67^{th} of the CH3 is referred to herein as Y407. Accordingly, the mutated aa residue can be added after the EU numbering in order to specify a mutation, such as S354C, E357W, Y349C, K370R, D356E, L358M, T366W, T366S, L368A, and Y407V.

In some embodiments, any one or more of the CH2 sequences comprise APEAAGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALKAPIEKTISKAK (SEQ ID NO:77; aa 6 to aa 115 of SEQ ID NO: 63, L234A, L235A, and P329K); or APELLGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAK (SEQ ID NO:78; aa 6 to aa 115 of SEQ ID NO: 62, L234, L235, and P329). In some embodiments, any one or more of the CH2 sequences lacks a mutation reducing (including significantly reducing and abolishing) an effector function of the multispecific binding agent. In further embodiments, any one or more of the CH2 sequences lacks any one or any two or all three of the following mutations: L234A, L235A, and P329K (accordingly to the EU numbering). In some embodiments, any one or more of the CH2 sequences comprise any one, or any two, or all three of the following: L234, L235, and P329 (accordingly to the EU numbering). In some embodiments, any one or more of the CH2 sequences lacks a mutation reducing an effector function of the multispecific binding agent. In some embodiments, the multispecific binding agent as described herein comprises one or more CH2 domains as disclosed in WO 2022/125986.

In some embodiments, one or more light chain constant domain (CL) comprises RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 79) or RTVAAPSVFIFPPSDSQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 67). In some embodiments, the multispecific antibody as described herein comprises one or more CL mutations as disclosed in WO 2022/125986. In some embodiments, the multispecific binding agent as described herein comprises one or more CL domains as disclosed in WO 2022/125986.

In some embodiments, one or more CH1 sequences comprise ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSC (SEQ ID NO: 80; aa 1 to aa 103 of SEQ ID NO: 65), ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKRVEPKSC (SEQ ID NO: 81), or ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDRKVEPKSC (SEQ ID NO: 82). In some embodiments, the multispecific binding agent as described herein comprises one or more CH1 mutations as disclosed in WO 2022/125986. In some embodiments, the multispecific binding agent as described herein comprises one or more CH1 domains as disclosed in WO 2022/125986.

In some embodiments, a hinge domain is immediately adjacent to the N terminus of a CH2 domain, for example, between a CH3 and a CH2, or between a light chain consistent domain (CL) and a CH2. Additionally or alternatively, a hinge domain is immediately adjacent to the C terminus of a CL and the N terminus of a light chain variable domain (VL). In further embodiments, the hinge domain comprises DKTHTCPPCP (SEQ ID NO:83). In some embodiments, the multispecific binding agent as described herein comprises one or more domain junctions as disclosed in WO 2022/125986.

In some embodiments, the multispecific antibody as described herein has an antibody construct as disclosed in WO 2022/125986.

In some aspect, provided herein is a binding agent comprising four polypeptide chains: a first polypeptide, a second polypeptide, a third polypeptide and a fourth polypeptide. In some embodiments, the first polypeptide comprises (i) an amino acid sequence of SEQ ID NO:48 or (ii) an amino acid sequence of SEQ ID NO: 48 lacking the C-terminal lysine (K). Additionally or alternatively, the second polypeptide comprises (i) an amino acid sequence of SEQ ID NO:49, or (ii) an amino acid sequence of SEQ ID NO:49 lacking the C-terminal lysine (K). Additionally or alternatively, the third polypeptide chain comprises (i) an amino acid sequence of SEQ ID NO:50, or (ii) an amino acid sequence of SEQ ID NO:50 lacking the C-terminal lysine (K). Additionally or alternatively, the fourth polypeptide chain comprises an amino acid sequence of SEQ ID NO:51.

In some embodiments, provided is a binding agent designated herein as bsAb1, i.e., a binding agent comprising a first polypeptide chain as set forth in SEQ ID NO: 48, a second polypeptide chain as set forth in SEQ ID NO: 49, a third polypeptide chain as set forth in SEQ ID NO: 50 and a fourth polypeptide chain as set forth in SEQ ID NO: 51. In further embodiments, the C-terminal lysine of the first polypeptide chain of bsAb1 is removed (such as, cleaved off) from the binding agent. Additionally or alternatively, the C-terminal lysine of the second polypeptide chain of bsAb1 is removed (such as, cleaved off) from the binding agent. Additionally or alternatively, the C-terminal lysine of the third polypeptide chain of bsAb1 is removed (such as, cleaved off) from the binding agent. Additionally or alternatively, the C-terminal lysine of the first polypeptide chain and C-terminal lysine of the second polypeptide chain of bsAb1 are removed (such as, cleaved off) from the binding agent. Additionally or alternatively, the C-terminal lysine of the first polypeptide chain and C-terminal lysine of the third polypeptide chain of bsAb1 are removed (such as, cleaved off) from the binding agent. Additionally or alternatively, the C-terminal lysine of the second polypeptide chain and C-terminal lysine of the third polypeptide chain of bsAb1 are removed (such as, cleaved off) from the binding agent. Additionally or alternatively, each C-terminal lysine of the first, second and third polypeptide chain is removed (such as, cleaved off) from the binding agent. As used herein, a bsAb1 having any one or any two or all three of its C-terminal lysine amino acid residues removed is referred to as a C-terminal variant of bsAb1. Accordingly, also provided herein is a composition comprising the bsAb1 and one or more C-terminal variants thereof, or a composition comprising one or more C-terminal variants of bsAb1. As it would be understood by one of skill in the art, a composition, a method, a use or any other embodiments relating to bsAb1 as disclosed herein also extend to a composition, a method, a use or an embodiment of (i) a C-terminal variant of bsAb1 or (ii) a composition comprising any one or more of: the bsAb1 and/or a C-terminal variant thereof.

In some embodiments, the multispecific antibody described herein is a monoclonal antibody. In some embodiments, the monoclonal antibody is a humanized, human or chimeric antibody. In some embodiments, the multispecific antibody described herein is a Fab, Fab′, F(ab′)₂, Fv, scFv, (scFv) 2, single chain antibody molecule, dual variable region antibody, single variable region antibody, linear antibody, V region, or a multispecific antibody formed from antibody fragments. In some embodiments, the multispecific antibody described herein is a recombinant antibody, which is optionally a humanized, human or chimeric antibody.

In some embodiments, a multispecific binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate a multispecific binding agent comprising a non-antibody protein scaffold (see, e.g., Simeon and Chen, Protein Cell, 9 (1): 3-14 (2018); Yang et al., Annu Rev Anal Chem (Palo Alto Calif). 10 (1): 293-320 (2017)).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10:1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67 (2015): 95-106. In some embodiments, an exemplary antibody format is a B-Body format for monospecific or multispecific (e.g., bispecific antibodies) as described in e.g. International Patent Application Publication Nos. WO 2018/075692, WO 2022/125986, and US Patent Application Publication No. 2018/0118811.

Bispecific antibody fragments are a format of bispecific antibody molecules that lack some or all of the antibody constant domains. For example, some bispecific antibody fragments lack an Fc region. In some embodiments, bispecific antibody fragments include heavy and light chain regions that are connected by a peptide linker that permits efficient expression of the bispecific antibody fragments in a single host cell. Non-limiting examples of bispecific antibody fragments include, but are not limited to, nanobody, nanobody-HAS, BITE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFV₂, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be used to extend the serum half-life of antibody fragments. In some embodiments, chemical conjugation, e.g., chemical conjugation of antibodies and/or antibody fragments, can be used to create bispecific antibody fragments molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, multispecific binding agents (e.g., antibodies, such as bispecific antibodies), including multispecific binding agents that bind to CD47, including human CD47, and one or more targets that are not CD47 (e.g., PD-L1, including human PD-L1), disclosed herein can be provided in any antibody format disclosed herein or known in the art. As a non-limiting example, in some embodiments, the multispecific binding agents (e.g., antibodies, such as bispecific antibodies), including multispecific binding agents that bind to CD47, including human CD47, and one or more targets that are not CD47 (e.g., PD-L1, including human PD-L1), can be selected from Fabs-in-tandem-Ig (FIT-Ig); DVD-Ig; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-Ig=dual variable domain immunoglobulin (Abbott); kappa-lambda bodies; TBTI=tetravalent bispecific tandem Ig; and CrossMab (Roche).

In some embodiments, a multispecific (e.g., bispecific) antibody disclosed herein comprises a CD47 binding domain and one or more additional binding domains that bind to one or more targets that are not CD47. In some embodiments, a multispecific (e.g., bispecific) antibody disclosed herein comprises a CD47 binding domain that comprises the VH and/or VL amino acid sequences of Table 1.

In some embodiments, described herein is a multispecific (e.g., bispecific) antibody comprising a binding domain which binds to CD47 and comprises VH and VL CDRs as set forth in Table 1.

In some embodiments, a multispecific (e.g., bispecific) antibody disclosed herein comprises a CD47 binding domain and PD-L1 binding domain. In some embodiments, a multispecific (e.g., bispecific) antibody disclosed herein comprises a CD47 binding domain that comprises the VH and/or VL amino acid sequences of Table 1 and a PD-L1 binding domain that comprises the VH and/or VL amino acid sequences of Table 2.

In some embodiments, a multispecific (e.g., bispecific) antibody disclosed herein comprises a CD47 binding domain that comprises the VH and VL amino acid sequences of Table 1 and a PD-L1 binding domain that comprises the VH and VL amino acid sequences of Table 2.

In some embodiments, the antibody is a human antibody, including, but not limited to, an antibody having variable regions in which both the framework and CDR regions are derived from human germline immunoglobulin sequences as described, for example, in Kabat et al. (1991) Sequences of proteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences, for example, to enhance the activity of the antibody, but do not comprise CDRs derived from other species (e.g., a mouse CDR placed within a human variable framework region).

Antibodies that bind CD47 and/or PD-L1 may be obtained by any suitable method, such as (but not limited to) immunization with whole tumor cells comprising CD47 and/or PD-L1 and collection of antibodies, recombinant techniques, or screening libraries of antibodies or antibody fragments using CD47 extracellular domain epitopes or PD-L1 extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human CD47 antigen and generating a hybridoma from spleen cells taken from the animal. A hybridoma may produce a monoclonal antibody or antibody fragment that binds CD47. One exemplary technique for generating monoclonal antibodies comprises immunizing an animal with a human PD-L1 antigen and generating a hybridoma from spleen cells taken from the animal. A hybridoma may produce a monoclonal antibody or antibody fragment that binds PD-L1.

In a further embodiment, monoclonal antibodies or antibody fragments can be isolated from antibody phage libraries generated using the techniques described in, for example, Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12:433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12:725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227:381-388 (1992).

Screening of the libraries can be accomplished by various techniques known in the art. For example, CD47 (e.g., a CD47 polypeptide, fragment or epitope) or PD-L1 (e.g., a PD-L1 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8:309-314 (1990) and in WO 92/09690, and a low coating density of antigen as described in Marks et al., Biotechnol., 10:779-783 (1992).

Multispecific binding agents can be obtained by designing a suitable antigen screening procedure to select for the phage clone of interest followed by construction of a full length multispecific binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind CD47 and/or PD-L1 may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8:455 58, 1997; Jakobovits et al., Ann. N. Y. Acad. Sci., 764:525 35, 1995; Green et al., Nature Genet., 7:13-21, 1994; Lonberg et al., Nature, 368:856-859, 1994; Taylor et al., Int. Immun. 6:579-591, 1994; and U.S. Pat. No. 5,877,397.

For example, human antibodies that bind CD47 and/or PD-L1 may be obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. International Patent Publication No. WO 96/30498 discloses the use of the Cre/Lox system to modify the immunoglobulin locus in a mammal, such as to replace all or a portion of the constant or variable region to form a modified antibody molecule. International Patent Publication No. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in International Patent Publication No. WO 96/33735. The monoclonal antibodies can be tested for the ability to inhibit or neutralize the biological activity or physiological effect of the corresponding protein.

### Fusion Proteins and Conjugates

The present disclosure provides multispecific binding agents (e.g., antibodies, such as bispecific antibodies), including multispecific binding agents that bind to CD47, including human CD47, and one or more targets that are not CD47 (e.g., PD-L1, including human PD-L1) with a masking moiety and/or cleavable moiety in which one or more of the CD47 and/or other target binding domains of the multispecific binding agent (e.g., an antibody) are masked (e.g., via a masking moiety) and/or activatable (e.g., via a cleavable moiety). Technologies for masking a multispecific binding agent (e.g., an antibody, such as a bispecific antibody) are well-known in the art, including SAFE body masking technology (see, e.g., US Patent Application Publication No. 2019/0241886) and Probody masking technology (see, e.g., US Patent Application Publication No. 2015/0079088). Such technologies can be used to generate a multispecific binding agent (e.g., an antibody, such as a bispecific antibody) that is masked and/or activatable. In some embodiments, such masked and/or activatable multispecific binding agents (e.g., antibodies, such as bispecific antibodies), including multispecific binding agents that bind to CD47, including human CD47, and one or more targets that are not CD47 (e.g., PD-L1, including human PD-L1), are useful for the preparation of conjugates, including immunoconjugates, antibody-drug conjugates (ADCs), masked ADCs and activatable antibody-drug conjugates (AADCs), comprising any one of the multispecific binding agents (e.g., antibodies, such as bispecific antibodies), including multispecific binding agents that bind to CD47, including human CD47, and one or more targets that are not CD47 (e.g., PD-L1, including human PD-L1), such as human CD47 binding agents, of the present disclosure. In some embodiments, multispecific binding agents (e.g., antibodies, such as bispecific antibodies), including multispecific binding agents that bind to CD47, including human CD47, and one or more targets that are not CD47 (e.g., PD-L1, including human PD-L1), such as human CD47 binding agents, of the present disclosure, are directly or indirectly linked another agent such as a drug. In further embodiments, a multispecific binding agent as described herein is covalently bound by a synthetic linker to one or more agents such as drugs.

If desired, a multispecific binding agent (e.g., an antibody, such as a bispecific antibody) is linked or conjugated (directly or indirectly) to a moiety with effector function, such as cytotoxic activity (e.g., a chemotherapeutic moiety or a radioisotope) or immune recruitment activity (e.g., a cytokine). Moieties that are linked or conjugated (directly or indirectly) include drugs that are cytotoxic (e.g., toxins such as aurostatins) or non-cytotoxic (e.g., signal transduction modulators such as kinases or masking moieties that mask one or more binding domains of a multispecific binding agent (e.g., an antibody, such as a bispecific antibody), or cleavable moieties that allow for activating a multispecific binding agent by cleaving a cleavable moiety to unmask one or more binding domains of a multispecific binding agent (e.g., an antibody, such as a bispecific antibody) in the tumor microenvironment) in the form of masked conjugates. Moieties that promote immune recruitment can include other antigen-binding agents, such as viral proteins that bind selectively to cells of the innate immune system. Alternatively or in addition, a multispecific binding agent (e.g., an antibody, such as a bispecific antibody) is optionally linked or conjugated (directly or indirectly) to a moiety that facilitates isolation from a mixture (e.g., a tag) or a moiety with reporter activity (e.g., a detection label or reporter protein). It will be appreciated that the features of a multispecific binding agent (e.g., an antibody, such as a bispecific antibody) described herein extend also to a polypeptide comprising a binding agent fragment.

In some embodiments, multispecific binding agents (e.g., antibodies, such as bispecific antibodies), including multispecific binding agents that bind to CD47, including human CD47, and one or more targets that are not CD47 (e.g., PD-L1, including human PD-L1) described herein may be linked or conjugated (directly or indirectly) to a polypeptide, which can result in the generation of an activatable antibody. In some embodiments, a multispecific binding agent (e.g., an antibody, such as a bispecific antibody) is linked or conjugated (directly or indirectly) to an agent. In some embodiments, the agent is a drug, resulting in an ADC or an AADC when the antibody of the ADC comprises a masking moiety and a cleavable moiety.

In some embodiments, multispecific binding agents (e.g., antibodies, such as bispecific antibodies), including multispecific binding agents that bind to CD47, including human CD47, and one or more targets that are not CD47 (e.g., PD-L1, including human PD-L1) described herein are conjugated or recombinantly linked (directly or indirectly) to a therapeutic agent (e.g., a cytotoxic agent) or to a diagnostic or detectable agent. The conjugated or recombinantly linked antibodies, including masked or activatable conjugates, can be useful, for example, for treating or preventing a disease or disorder such as an immune cell dysfunctional disease, disorder or condition. The conjugated or recombinantly linked multispecific binding agents (e.g., antibodies, such as bispecific antibodies), including multispecific binding agents that bind to CD47, including human CD47, and one or more targets that are not CD47 (e.g., PD-L1, including human PD-L1), including masked or activatable conjugates, can be useful, for example, for monitoring or prognosing the onset, development, progression, and/or severity of an immune cell dysfunctional disease.

Such diagnosis and detection can be accomplished, for example, by coupling the multispecific binding agent (e.g., an antibody, such as a bispecific antibody) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also described herein are multispecific binding agents (e.g., antibodies, such as bispecific antibodies) that are recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In particular, described herein are fusion proteins comprising an antigen-binding fragment of a multispecific binding agent (e.g., an antibody, such as a bispecific antibody), including a multispecific binding agent that binds to CD47, including human CD47, and one or more targets that are not CD47 (e.g., PD-L1, including human PD-L1), described herein (e.g., comprising CDR1, CDR2, and/or CDR3 of VH and/or VL) and a heterologous protein, polypeptide, or peptide. In some embodiments, the heterologous protein, polypeptide, or peptide that a multispecific binding agent (e.g., an antibody, such as a bispecific antibody) is linked to is useful for targeting the multispecific binding agent to a particular cell (e.g., a CD47 expressing cell and/or PD-L1 expressing cell, including a tumor cell).

Moreover, multispecific binding agents (e.g., antibodies, such as bispecific antibodies), including multispecific binding agents that bind to CD47, including human CD47, and one or more targets that are not CD47 (e.g., PD-L1, including human PD-L1), described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a multispecific binding agent (e.g., an antibody) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody (e.g., a non-CD47 binding antibody or a non-PD-L1 binding antibody)). In certain embodiments, the fusion protein retains the biological activity of a multispecific binding agent. In certain embodiments, the fusion protein comprises a first binding domain that comprises CD47 antibody VH region, VL region, VH CDR (one, two or three VH CDRs), and/or VL CDR (one, two or three VL CDRs), wherein the fusion protein binds to a CD47 epitope, a CD47 fragment and/or a CD47 polypeptide. In certain embodiments, the fusion protein comprises a second binding domain that comprises PD-L1 antibody VH region, VL region, VH CDR (one, two or three VH CDRs), and/or VL CDR (one, two or three VL CDRs), wherein the fusion protein binds to a PD-L1 epitope, a PD-L1 fragment and/or a PD-L1 polypeptide.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the multispecific binding agents (e.g., antibodies, such as bispecific antibodies), including multispecific binding agents that bind to CD47, including human CD47, and one or more targets that are not CD47 (e.g., PD-L1, including human PD-L1), as described herein, including, for example, multispecific binding agents with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16 (2): 76-82; Hansson et al., 1999, J. Mol. Biol. 287:265-76; and Lorenzo and Blasco, 1998, Biotechniques 24 (2): 308-13). In some embodiments, multispecific binding agents, including multispecific binding agents that bind to CD47, including human CD47, and one or more targets that are not CD47 (e.g., PD-L1, including human PD-L1), may be altered by being subjected to random mutagenesis by error-prone PCR, random nucleotide insertion, or other methods prior to recombination. A polynucleotide encoding a multispecific binding agent described herein may be recombined with one or more components, motifs, sections, parts, domains, fragments, etc. of one or more heterologous molecules.

Multispecific binding agents (e.g., antibodies, such as bispecific antibodies), including multispecific binding agents that bind to CD47, including human CD47, and one or more targets that are not CD47 (e.g., PD-L1, including human PD-L1), described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

Multispecific binding agents (e.g., antibodies, such as bispecific antibodies), including multispecific binding agents that bind to CD47, including human CD47, and one or more targets that are not CD47 (e.g., PD-L1, including human PD-L1), described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., antibody-drug conjugates) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of an ADC or an AADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents, including wherein the agent is a drug for the preparation of an ADC or an AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of an ADC or an AADC, have been based on random conjugation chemistries involving the s-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332:41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48 (50): 12047-57).

In some embodiments, a multispecific binding agent (e.g., an antibody, such as a bispecific antibody), including a multispecific binding agent that binds to CD47, including human CD47, and one or more targets that are not CD47 (e.g., PD-L1, including human PD-L1), described herein is conjugated to a cytotoxic agent. In some embodiments, a multispecific binding agent (e.g., an antibody, such as a bispecific antibody), including a multispecific binding agent that binds to CD47, including human CD47, and one or more targets that are not CD47 (e.g., PD-L1, including human PD-L1), disclosed herein can be optionally conjugated with one or more cytotoxic agent(s) disclosed herein or known in the art in order to generate an ADC or an AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a multispecific binding agent (e.g., an antibody, such as a bispecific antibody), including a multispecific binding agent that binds to CD47, including human CD47, and one or more targets that are not CD47 (e.g., PD-L1, including human PD-L1), described herein is conjugated to a drug such as a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In some embodiments, the mitotic inhibitor is a dolastatin, an auristatin, a maytansinoid, and a plant alkaloid. In some embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. In some embodiments, the actinomycine is a pyrrolobenzodiazepine (PBD).

### Polynucleotides, Vectors and Production

Further provided are the materials for generating mutispecific binding agents and fragments thereof. For example, an isolated cell (e.g., a hybridoma) may produce a multispecific binding agent (e.g., antibody or antibody fragment). In this regard, a cell (e.g., an isolated cell) may produce an antibody or fragment thereof comprising a first binding domain comprising a VH and a VL as shown in Table 1 for mAb-C. Alternatively or additionally, a cell (e.g., an isolated cell) may produce an antibody or fragment thereof comprising a second binding domain comprising a VH and a VL as shown in Table 2 for mAb-P.

In some embodiments, provided herein is a polynucleotide encoding a multispecific binding agent as disclosed herein, a polypeptide chain thereof, or a fusion polypeptide as disclosed herein, or a polynucleotide complementary thereto. In some embodiments, polynucleotides described herein may comprise one or more nucleic acid sequences encoding the multispecific binding agent (e.g., antibody or antibody fragment). In some embodiments, the polynucleotide is an isolated and/or recombinant polynucleotide. In various aspects, the isolated polynucleotide comprises a nucleotide sequence that encodes an antibody heavy chain variable region (VH) and/or an antibody light chain variable region (VL), wherein the VH and the VL comprise complementarity determining regions (CDRs) identical to CDRs as shown in Table 1. In various aspects, the isolated polynucleotide comprises a nucleotide sequence that encodes an antibody heavy chain variable region (VH) and/or an antibody light chain variable region (VL), wherein the VH and the VL comprise complementarity determining regions (CDRs) identical to CDRs as shown in Table 2.

As used herein, the term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present. The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

In some embodiments, one or more vectors (e.g., expression vectors) may comprise one or more polynucleotides as disclosed herein, such as, for expression of the one or more polynucleotides in a suitable host cell, and/or for producing the one or more polynucleotides as disclosed herein. Such vectors are useful, e.g., for amplifying the polynucleotides in host cells to create useful quantities thereof, and/or for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind CD47 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs as disclosed herein can also include sequences necessary for replication in a host cell.

Exemplary expression control sequences include promoter/enhancer sequences, e.g., cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86 (5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132:2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91:2687-2692, 1995; and Isner et al., Human Gene Therapy, 7:989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72 (1): 811-816, 1998; Kingsman & Johnson, Scrip Magazine, October 1998, pp. 43-46); parvoviral vectors, such as adeno-associated viral (AAV) vectors (U.S. Pat. Nos. 5,474,935l; 5,139,941; 5,622,856; 5,658,776; 5,773,289; 5,789,390; 5,834,441; 5,863,541; 5,851,521; 5,252,479; Gnatenko et al., J. Invest. Med., 45:87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89:2581-2584, 1992; Stratford Perricaudet et al., J. Clin. Invest., 90:626-630, 1992; and Rosenfeld et al., Cell, 68:143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985, DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am., 276 (6): 102-6, 1997; Felgner, Hum Gene Ther., 7 (15): 1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

An expression vector (or the antibody or fragment thereof discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275 (5301): 810-814, 1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In some embodiments, the liposome is complexed or employed in conjunction with nuclear nonhistone chromosomal proteins (HMG-1) (Kato et al., J. Biol. Chem., 266:3361-3364, 1991). In some embodiments, the liposomes are complexed or employed in conjunction with both HVJ and HMG-1. Such expression constructs have been successfully employed in transfer and expression of nucleic acid in vitro and in vivo. In some embodiments, a multispecific binding agent (e.g., an antibody), including a multispecific binding agent that binds to CD47, including human CD47, and one or more targets that are not CD47 (e.g., PD-L1, including human PD-L1), is included in the liposome to target the liposome to cells (such as tumor cells) expressing CD47 and/or PD-L1 on their surface.

Additionally provided are a cell comprising any one or more of: a binding agent as disclosed herein, a nucleic acid as disclosed herein, or a vector as disclosed herein. In some embodiments, the cell expresses the binding agent provided herein. In some embodiments, the cell replicates the nucleic acid or the vector.

A cell may comprise one or more polynucleotides or one or more vectors, e.g., the cell is transformed or transfected with one or more polynucleotides encoding a multispecific binding agent (e.g., an antibody), including a multispecific binding agent that binds to CD47, including human CD47, and one or more targets that are not CD47 (e.g., PD-L1, including human PD-L1), or the one or more vectors comprising the one or more polynucleotides. In some embodiments, cells express a multispecific binding agent, including a multispecific binding agent that binds to CD47, including human CD47, and one or more targets that are not CD47 (e.g., PD-L1, including human PD-L1), having at least 75% identity to the CDRs of mAb-C (see, e.g., Table 1). In some embodiments, cells express a multispecific binding agent, including a multispecific binding agent that binds to CD47, including human CD47, and PD-L1, including human PD-L1, having at least 75% identity to the CDRs of mAb-P (see, e.g., Table 2). The cells may be prokaryotic cells, such as Escherichia coli (see, e.g., Pluckthun et al., Methods Enzymol., 178:497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., multispecific binding agents (e.g., antibodies, such as bispecific antibodies), including multispecific binding agents that bind to CD47, including human CD47, and one or more targets that are not CD47 (e.g., PD-L1, including human PD-L1)) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a multispecific binding agent (e.g., an antibody) may comprise culturing a host cell and isolating the multispecific binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3^{rd} ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, U K, 1992); and Current Protocols in Molecular Biology, Ausubel ed., Wiley Interscience, New York, 1999).

A multispecific binding agent (e.g., an antibody), including a multispecific binding agent that binds to CD47, including human CD47, and one or more targets that are not CD47 (e.g., PD-L1, including human PD-L1), is produced using any suitable method, e.g., isolated from an immunized animal, recombinantly or synthetically generated, or genetically-engineered, including as described above. Antibody fragments derived from an antibody are obtained by, e.g., proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1:422 Academic Press (1967); Nisonoff et al., Arch. Biochem. Biophys., 89:230-244, 1960; Porter, Biochem. J., 73:119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.1 2.10A.5.

### Uses and Methods

In another aspect, provided herein are methods for using the binding agents such as the multispecific binding agents or the composition provided herein.

In some embodiments, provided herein is a method of inhibiting interaction between CD47 (for example, expressed on and/or in a first cell) and SIRPα (for example, expressed on and/or in a second cell), comprising contacting CD47 (for example, the first cell expressing CD47) with a multispecific binding agent (e.g., a multispecific antibody or fragment thereof) provided herein. In some embodiments, provided herein is a use of the multispecific binding agent provided herein for inhibiting interaction between CD47 (for example, expressed on and/or in a first cell) and SIRPα (for example, expressed on and/or in a second cell), wherein the use comprises contacting CD47 (for example, the first cell expressing CD47) with a multispecific binding agent (e.g., the antibody or fragment thereof) provided herein.

In some embodiments of any method or any use as disclosed herein, the multispecific binding agent does not substantially inhibit interaction between CD47 expressing on a red blood cell and SIRPα expressing on an immune cell (such as, a macrophage, a neutrophil, a dendritic cell, or a B lymphocyte). In further embodiments, the multispecific binding agent does not inhibit interaction between CD47 expressing on a red blood cell and SIRPα expressing on an immune cell (such as, a macrophage, a neutrophil, a dendritic cell, or a B lymphocyte). In other embodiments, the multispecific binding agent inhibits interaction between CD47 expressing on a red blood cell and SIRPα expressing on an immune cell (such as, a macrophage, a neutrophil, a dendritic cell, or a B lymphocyte), but the inhibition is significantly less (for example, at least about 10%, at least about 20%, at least about 30%, at least about 40%, at least about 50%, at least about 60%, at least about 70%, at least about 80%, at least about 90%, or at least about 95% less) than that of a benchmark anti-CD47 antibody.

As used herein, the term “inhibit” means decrease or reduce. It can refer to a 100% elimination as well as any decrease or reduction, such as a significantly decrease or reduction (for example, by at least about 10%, by at least about 20%, by at least about 30%, by at least about 40%, by at least about 50%, by at least about 60%, by at least about 70%, by at least about 80%, by at least about 90%, or by at least about 95%). For example, in some embodiments, the binding agent provided herein inhibits the interaction between CD47 (or an extracellular domain thereof) and SIRPα by 10%-99%. In other embodiments, the binding agent provided herein inhibits the interaction between CD47 (or an extracellular domain thereof) and SIRPα 100% (e.g., completely abolish the interaction as measured by an assay). In some embodiments, the binding agent provided herein inhibits the interaction between CD47 (or an extracellular domain thereof) and SIRPα by at least 10%. In some embodiments, the binding agent provided herein inhibits the interaction between CD47 (or an extracellular domain thereof) and SIRPα by at least 20%. In some embodiments, the binding agent provided herein inhibits the interaction between CD47 (or an extracellular domain thereof) and SIRPα by at least 30%. In some embodiments, the binding agent provided herein inhibits the interaction between CD47 (or an extracellular domain thereof) and SIRPα by at least 40%. In some embodiments, the binding agent provided herein inhibits the interaction between CD47 (or an extracellular domain thereof) and SIRPα by at least 50%. In some embodiments, the binding agent provided herein inhibits the interaction between CD47 (or an extracellular domain thereof) and SIRPα by at least 60%. In some embodiments, the binding agent provided herein inhibits the interaction between CD47 (or an extracellular domain thereof) and SIRPα by at least 70%. In some embodiments, the binding agent provided herein inhibits the interaction between CD47 (or an extracellular domain thereof) and SIRPα by at least 80%. In some embodiments, the binding agent provided herein inhibits the interaction between CD47 (or an extracellular domain thereof) and SIRPα by at least 90%. In some embodiments, the binding agent provided herein inhibits the interaction between CD47 (or an extracellular domain thereof) and SIRPα by about 10%-90%. In some embodiments, the binding agent provided herein inhibits the interaction between CD47 (or an extracellular domain thereof) and SIRPα by about 20%-80%. In some embodiments, the binding agent provided herein inhibits the interaction between CD47 (or an extracellular domain thereof) and SIRPα by about 30%-70%. In some embodiments, the binding agent provided herein inhibits the interaction between CD47 (or an extracellular domain thereof) and SIRPα by about 40%-60%.

In some embodiments, CD47 and SIRPα are expressed on different cells. In some embodiments, the CD47 is expressed on a first cell, such as a tumor cell. In further embodiments, the first cell is not a red blood cell. In yet a further embodiment, the first cell is not a progenitor of a red blood cell. Additionally or alternatively, the SIRPα is expressed on a second cell, such as an immune cell. In further embodiments, the immune cell is a macrophage, a neutrophil, a dendritic cell, or a B lymphocyte.

In some embodiments, provided herein is a method of inhibiting interaction between PD-1 (for example, expressed on and/or in a first cell) and PD-L1 (for example, expressed on and/or in a second cell), comprising contacting PD-L1 (and/or the second cell expressing the PD-L1) with a binding agent (e.g., a multispecific binding agent, a multispecific antibody or fragment thereof) provided herein. In some embodiments, provided herein is a use of the binding agent (e.g., a multispecific binding agent, a multispecific antibody or fragment thereof) provided herein for inhibiting interaction between PD-1 (for example, expressed on and/or in a first cell) and PD-L1 (for example, expressed on and/or in a second cell), wherein the use comprises contacting PD-L1 (and/or the second cell expressing the PD-L1) with a binding agent (e.g., a multispecific binding agent, the multispecific antibody or fragment thereof) provided herein.

For example, in some embodiments, the binding agent provided herein inhibits the interaction between PD-L1 and PD-1 by 10%-99%. In other embodiments, the binding agent provided herein inhibits the interaction between PD-L1 and PD-1 by 100% (e.g., completely abolish the interaction as measured by an assay). In some embodiments, the binding agent provided herein inhibits the interaction between PD-L1 and PD-1 by at least 10%. In some embodiments, the binding agent provided herein inhibits the interaction between PD-L1 and PD-1 by at least 20%. In some embodiments, the binding agent provided herein inhibits the interaction between PD-L1 and PD-1 by at least 30%. In some embodiments, the binding agent provided herein inhibits the interaction between PD-L1 and PD-1 by at least 40%. In some embodiments, the binding agent provided herein inhibits the interaction between PD-L1 and PD-1 by at least 50%. In some embodiments, the binding agent provided herein inhibits the interaction between PD-L1 and PD-1 by at least 60%. In some embodiments, the binding agent provided herein inhibits the interaction between PD-L1 and PD-1 by at least 70%. In some embodiments, the binding agent provided herein inhibits the interaction between PD-L1 and PD-1 by at least 80%. In some embodiments, the binding agent provided herein inhibits the interaction between PD-L1 and PD-1 by at least 90%. In some embodiments, the binding agent provided herein inhibits the interaction between PD-L1 and PD-1 by about 10%-90%. In some embodiments, the binding agent provided herein inhibits the interaction between PD-L1 and PD-1 by about 20%-80%. In some embodiments, the binding agent provided herein inhibits the interaction between PD-L1 and PD-1 by about 30%-70%. In some embodiments, the binding agent provided herein inhibits the interaction between PD-L1 and PD-1 by about 40%-60%.

In some embodiments, PD-1 and PD-L1 are expressed on different cells. In some embodiments, the PD-1 cell is expressed on a first cell, such as an immune cell. In further embodiments, the immune cell is a T cell. In yet further embodiments, the T cell is a cytotoxic T cell such as a CD8+ T cell. In som embodiments, the immune cell is an NK cell. Additionally or alternatively, the PD-L1 is expressed on a second cell, such as a tumor cell.

An immune cell is a cell in the immune system and can be a cell of lymphoid lineage. Non-limiting examples of cells of lymphoid lineage include neutrophils, eosinophils, basophils, mast cells, monocytes, macrophages, dendritic cells, natural killer (NK) cells, and lymphocytes (B cells and T cells). T cells are a type of lymphocytes and can be characterized by expressing T cell receptors (TCRs). T cells play a central role in the adaptive immune response. T cell subtypes have a variety of important functions in controlling and shaping the immune response. For example, cytotoxic T cells (also called cytotoxic T lymphocyte and killer T cell) are T lymphocytes that kill certain cells, e.g., tumor cells, cells that are infected by intracellular pathogens (such as viruses or bacteria), or cells that are damaged in other ways. Most cytotoxic T cells express T-cell receptors (TCRs) that can recognize a specific antigen. CD8+ T cells are a subpopulation of MHC class I-restricted T cell and are mediators of adaptive immunity, which are important for killing cancerous or virally infected cells. NK cells are a type of cytotoxic lymphocytes critical to the innate immune system that belong to the family of innate lymphoid cells (ILC). In some embodiments, NK cells can be identified by the presence of CD56 and the absence of CD3 (CD56+, CD3−). NK cells have the ability to recognize and kill stressed cells in the absence of antibodies and MHC, allowing for a much faster immune reaction.

In some embodiments, provided herein is a method of inhibiting interaction between CD47 (or an extracellular domain thereof) (for example, expressed on and/or in a first cell) and SIRPα (for example, expressed on and/or in a second cell) and inhibiting interaction between PD-1 (for example, expressed on and/or in a third cell) and PD-L1 (for example, expressed on and/or in a fourth cell), comprising contacting CD47 (or an extracellular domain of each thereof, or the first cell expressing CD47) and PD-L1 (and/or the fourth cell expressing the PD-L1) with a binding agent (e.g., a multispecific binding agent, a multispecific antibody or fragment thereof) provided herein. In some embodiments, provided herein is a use of the binding agent (e.g., a multispecific binding agent, a multispecific antibody or fragment thereof) provided herein for inhibiting interaction between CD47 (or an extracellular domain thereof) (for example, expressed on and/or in a first cell) and SIRPα (for example, expressed on and/or in a second cell) and inhibiting interaction between PD-1 (for example, expressed on and/or in a third cell) and PD-L1 (for example, expressed on and/or in a fourth cell), wherein the use comprises contacting CD47 (or an extracellular domain of each thereof, or the first cell expressing CD47) and PD-L1 (and/or the fourth cell expressing the PD-L1) with a binding agent (e.g., a multispecific binding agent, a multispecific antibody or fragment thereof) provided herein. In some embodiments, CD47 (or an extracellular domain thereof) and PD-L1 are expressed on and/or in the same cell (e.g., the first cell and the fourth cell are the same cell), such as a cancer or tumor cell. Additionally or alternatively, the cell expressing CD47 (or an extracellular domain thereof) is not a red blood cell. In yet a further embodiment, the cell expressing CD47 (or an extracellular domain thereof) is not a progenitor of a red blood cell. Additionally or alternatively, SIRPα and PD-1 are expressed on and/or in the same cell (e.g., the second cell and the third cell are the same cell), such as an immune cell. In some embodiments, SIRPα and PD-1 are expressed on and/or in different cells, for example, different immune cells. In one embodiment, SIRPα is expressed on a macrophage and PD-L1 is expressed on T cell (such as, a cytotoxic T cell) or an NK cell.

In further embodiments, the method lacks inhibiting interaction between CD47 (or an extracellular domain thereof) substantially expressed in or on a red blood cell (RBC) and SIRPα (for example, expressed on and/or in a second cell). In other embodiments, the method inhibits interaction between CD47 (or an extracellular domain thereof) substantially expressed in or on a red blood cell (RBC) and SIRPα (for example, expressed on and/or in a second cell), but the inhibition is significantly (for example, at least about 20%, at least about 30%, at least about 40%, at least about 50%, at least about 60%, at least about 70%, at least about 80%, at least about 90%, or at least about 95%) less than that of a benchmark anti-CD47 antibody. In one embodiment, SIRPα is expressed on an immune cell, such as a macrophage. In some embodiments, the RBC does not substantially express PD-L1.

In some embodiments, provided herein is a method of preventing (e.g., reducing or decreasing, including but not limited to abolishing) or inhibiting suppression of an immune cell, such as a tumor/cancer associated immune suppression. The method comprises (i) inhibiting interaction between CD47 (or an extracellular domain thereof) (for example, expressed on and/or in a first cell) and SIRPα (for example, expressed on and/or in a second cell) and (ii) inhibiting interaction between PD-1 (for example, expressed on and/or in a third cell) and PD-L1 (for example, expressed on and/or in a fourth cell). In some embodiments, the method comprises contacting CD47 (or extracellular domain of each thereof, or the first cell expressing CD47) and PD-L1 (and/or the fourth cell expressing the PD-L1) with a binding agent (e.g., a multispecific binding agent, a multispecific antibody or fragment thereof) provided herein. In some embodiments, CD47 (or an extracellular domain thereof) and PD-L1 are expressed on and/or in the same cell (e.g., the first cell and the fourth cell are the same cell), such as a cancer or tumor cell. Additionally or alternatively, SIRPα and PD-1 are expressed on and/or in the same cell (e.g., the second cell and the third cell are the same cell), such as an immune cell. In some embodiments, SIRPα and PD-1 are expressed on and/or in different cells, for example, different immune cells. In one embodiment, SIRPα is expressed on a macrophage and PD-L1 is expressed on T cell (such as, a cytotoxic T cell) or an NK cell.

In other embodiments, provided herein is a method of preventing or inhibiting suppression of an immune cell, e.g., a suppression mediated by the interaction between SIRPα expressed on the immune cell with CD47 (or an extracellular domain thereof) (e.g., expressed on a cancer or tumor cell), and/or a suppression mediated by the interaction between PD-1 (e.g., expressed on the immune cell) and PD-L1 (e.g., expressed on a cancer or tumor cell). In yet other embodiments, provided herein is a method of activating a response mediated by an immune cell, e.g., an anti-tumor response. In some embodiments, the immune cell suppression is a tumor/cancer associated immune cell suppression, such as in a tumor microenvironment. Additionally or alternatively, the immune cell suppression is a suppression of an ADCP and/or an ADCC. In some embodiments, the method comprises contacting the immune cell with a binding agent (e.g., a multispecific antibody or fragment thereof) provided herein. Additionally or alternatively, the method comprises contacting the tumor cell with a binding agent (e.g., a multispecific antibody or fragment thereof) provided herein. In some embodiments, provided herein is a use of the binding agent provided herein for preventing suppression of an immune cell or activating a response mediated by an immune cell. In some embodiments, the immune cell is a macrophage, a neutrophil, a dendritic cell, or a B lymphocyte. In some embodiments, the immune cell is an NK cell. In some embodiments, the immune cell is a T cell. In some embodiments, the T cell is a cytotoxic T cell such as a CD8+ T cell. In some embodiments, the immune cell expresses SIRPα. Additionally or alternatively, the immune cell expresses PD-1. In some embodiments, the immune cell mediates an anti-cancer/tumor response. Additionally or alternatively, the immune cell mediates an ADCC and/or an ADCP. In further embodiments, the cancer or tumor cell expresses PD-L1. Additionally or alternatively, the cancer or tumor cell expresses CD47.

In some embodiments, the immune suppression (also referred to herein as suppression of an immune cell) is a suppression of an antibody-dependent cellular cytotoxicity (ADCC). In further embodiments, the ADCC is mediated by the PD-L1/PD-1 signaling. In some embodiments, the ADCC is mediated by T cells, such as T cells expressing PD-1. Additionally or alternatively, the immune suppression is a suppression of an antibody-dependent cellular phagocytosis (ADCP). In further embodiments, the ADCP is mediated by the CD47/SIRPα signaling. In some embodiments, the ADCP is mediated by macrophages, such as macrophages expressing SIRPα.

In one aspect, provided herein is a method of inducing ADCC of a tumor cell. In further embodiments, the tumor cell expresses PD-L1. In some embodiments, the method comprises contacting the tumor cell with a multispecific binding agent as disclosed herein. In some embodiments, the method comprises administering a multispecific binding agent or a composition as disclosed herein to a subject having the tumor cell. Also provided herein is a use of the multispecific binding agent provided herein for inducing ADCC of a tumor cell. In some embodiments, the use comprises contacting the tumor cell with a multispecific binding agent as disclosed herein. In some embodiments, the use comprises administering a multispecific binding agent or a composition as disclosed herein to a subject having the tumor cell. In further embodiments, the tumor cell expresses PD-L1.

In another aspect, provided herein is a method of inducing ADCP of a tumor cell. In further embodiments, the tumor cell expresses CD47. In some embodiments, the method comprises contacting the tumor cell with a multispecific binding agent ad disclosed herein. In some embodiments, the method comprises administering a multispecific binding agent or a composition as disclosed herein to a subject having the tumor cell. Also provided herein is a use of the multispecific binding agent provided herein for inducing ADCP of a tumor cell. In some embodiments, the use comprises contacting the tumor cell with a multispecific binding agent as disclosed herein. In some embodiments, the use comprises administering a multispecific binding agent or a composition as disclosed herein to a subject having the tumor cell. In further embodiments, the tumor cell expresses CD47.

In yet another aspect, provided herein is a method of inducing ADCC and ADCP of tumor cells and/or a method of reducing suppression of ADCC and ADCP of tumor cells. In some embodiments, the method comprises contacting the tumor cell with a multispecific binding agent as disclosed herein. In some embodiments, the method comprises administering a multispecific binding agent or a composition as disclosed herein to a subject having the tumor cell. Also provided herein is a use of the multispecific binding agent provided herein for inducing ADCC and ADCP of tumor cells and/or a method of reducing suppression of ADCC and ADCP of tumor cells. In some embodiments, the use comprises contacting the tumor cell with a multispecific binding agent as disclosed herein. In some embodiments, the use comprises administering a multispecific binding agent or a composition as disclosed herein to a subject having the tumor cell.

In further embodiments, each of the tumor cells expresses PD-L1 and CD47. In other embodiments, a first population (for example, at least 10%, or at least 20%, or at least 30%, or at least 40%, or at least 50%, or at least 60%, or at least 70%, or at least 80%, or at least 90%, or at least 95%) of the tumor cells expresses PD-L1, and a second population (for example, at least 10%, or at least 20%, or at least 30%, or at least 40%, or at least 50%, or at least 60%, or at least 70%, or at least 80%, or at least 90%, or at least 95%) of the tumor cells expresses CD47. In further embodiments, a significant percentage (for example, at least 80%, or at least 90%, or at least 95%, including 100%) of the tumor cells expresses PD-L1, or CD47, or both.

In some embodiments, a method as disclosed herein is an in vitro or ex vivo method. In other embodiments, a method as disclosed herein is an in vivo method. In some embodiments, a use as disclosed herein is an in vitro or ex vivo use. In other embodiments, a use as disclosed herein is an in vivo use.

Additionally provided herein is a method of treating a disease, disorder, or condition (such as those disclosed herein) in a subject in need thereof. In some embodiments, the method comprises administering to the subject an effective amount of (i) first means for inhibiting the interaction between PD-L1 and PD-1, and (ii) second means for inhibiting the interaction between CD47 and SIRPα. Also provided herein is a use of the multispecific binding agent provided herein for treating a disease, disorder, or condition (such as those disclosed herein) in a subject in need thereof. In some embodiments, the use comprises administering to the subject an effective amount of (i) first means for inhibiting the interaction between PD-L1 and PD-1, and (ii) second means for inhibiting the interaction between CD47 and SIRPα.

In some embodiments, the first means has a high affinity to PD-L1 (such as a human PD-L1 or a cyno PD-L1). In further embodiments, the first means bind to PD-L1 (such as a human PD-L1 or a cyno PD-L1) competitively with a multispecific binding agent as disclosed herein, such as bsAb1. Additionally or alternatively, the first means has a K_{D} for its interaction with PD-L1 of lower than 1 nM, or lower than 0.1 nM, such as about 0.05 nM.

Additionally or alternatively, the second means has a detuned and/or moderate affinity to CD47 (such as a human CD47 or a cyno CD47). In some embodiments, the second means bind to CD47 (such as a human Cd47 or a cyno CD47) competitively with a multispecific binding agent as disclosed herein, such as bsAb1. Additionally or alternatively, the second means has a K_{D} for its interaction with CD47 of higher than 0.1 nM, or higher than 1 nM, or higher than 5 nM, or higher than 10 nM, or higher than 15 nM, or higher than 20 nM. In further embodiments, the second means has a K_{D} for its interaction with CD47 of lower than 1 μM, or lower than 500 nM, or lower than 100 nM, or lower than 50 nM, or lower than 30 nM. In some embodiments, the second means has a K_{D} for its interaction with CD47 of about 0.1 nM to about 1 μM, including each number and range therebetween, such as about 1 nM to about 100 nM, or about 10 nM to about 50 nM, or about 20 to about 30 nM. In some embodiments, the method or use comprises administering a binding agent as disclosed herein or a composition as disclosed herein. In some embodiments, the K_{D} is measured by a method as disclosed herein, such as Biacore.

In some embodiments, multispecific binding agents (e.g., antibodies), including multispecific binding agents that bind to CD47, including human CD47, and one or more targets that are not CD47 (e.g., PD-L1, including human PD-L1) described herein are useful in compositions and in methods of treating, preventing, or alleviating an immune cell dysfunctional disease, disorder or condition (e.g., a phagocytic cell dysfunctional disease, disorder, or condition or a T cell dysfunctional disease, disorder or condition), including one or more symptoms of the disease, disorder, or condition. Phagocytic cell dysfunctional diseases, disorders, and conditions include tumor immunity and associated cancers, including, but not limited to, any cancer wherein the tumor cells express or overexpress CD47. T cell dysfunctional diseases, disorders, and conditions include tumor immunity and associated cancers, including, but not limited to, any cancer wherein the tumor cells express or overexpress PD-L1. Such CD47 and/or PD-L1 expressing tumor cells may help tumor cells escape immune surveillance and clearance (e.g., tumor immunity). In addition, multispecific binding agents described herein, such as multispecific antibodies (e.g., antibodies, such as bispecific antibodies), that bind to CD47 and one or more additional targets that are not CD47 (e.g., PD-L1), are useful to inhibit SIRPα signaling and/or PD-1 signaling, enhance phagocytic cell function and/or immune surveillance, and enhance removal of tumor cells.

In some embodiments, described herein is a method for treating tumor immunity in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., antibody) described herein.

In some embodiments, described herein is a method for treating a cancer or a tumor in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., antibody) described herein.

In some embodiments, described herein is a method for alleviating one or more symptoms associated with a cancer or a tumor in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., antibody) described herein.

In some embodiments, described herein is a method for decreasing tumor size in a subject with a tumor comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein.

In some embodiments, described herein is a method for enhancing tumor cell removal in a subject with a tumor comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein.

In some embodiments, described herein is a method for treating a phagocytic cell dysfunctional disease, disorder or condition in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein.

In some embodiments, described herein is a method for increasing immune cell phagocytosis in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein. In some embodiments, the immune cell is a macrophage, a neutrophil, a dendritic cell, or a B lymphocyte. In some embodiments, the subject is diagnosed with a cancer or a tumor.

In some embodiments, described herein is a method for treating a T cell dysfunctional disease, disorder or condition in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein. In some embodiments, the T cell dysfunctional disease, disorder or condition is tumor immunity.

In some embodiments, described herein is a method for enhancing T cell function in a subject comprising administering to the subject a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein. In some embodiments, the T cell function is secretion of cytokines. In some embodiments, the T cell function is removal of tumor cells. In some embodiments, the subject is diagnosed with a cancer or a tumor.

The subject of a method described above can be administered one or more therapeutic agents described herein in combination with a multispecific binding agent (e.g., an antibody), including a multispecific binding agent that binds to CD47, including human CD47, and one or more targets that are not CD47 (e.g., PD-L1, including human PD-L1), described herein or fragment thereof or a pharmaceutical composition comprising the binding agent (e.g., an antibody) described herein.

In some embodiments, a multispecific binding agent (e.g., an antibody), including a multispecific binding agent that binds to CD47, including human CD47, and one or more targets that are not CD47 (e.g., PD-L1, including human PD-L1), increases phagocytosis and/or enhances phagocytic activity of cells in cell culture. For example, such cell culture may include tumor cells expressing or overexpressing CD47. In some embodiments, a multispecific binding agent (e.g., an antibody), including a multispecific binding agent that binds to CD47, including human CD47, and PD-L1, including human PD-L1, increases T cell function and/or enhances cytolytic activity of cells in cell culture. Such cell culture may include tumor cells expressing or overexpressing PD-L1. In some embodiments, a multispecific binding agent (e.g., an antibody), including a multispecific binding agent that binds to CD47, including human CD47, and PD-L1, including human PD-L1, increases phagocytosis, enhances phagocytic activity, increases T cell function and/or enhances cytolytic activity of cells in cell culture. Such cell culture may include tumor cells expressing or overexpressing CD47 and PD-L1. Tumor cells include, but are not limited to, breast cancer cells, bladder cancer cells, melanoma cells, prostate cancer cells, mesothelioma cells, lung cancer cells, testicular cancer cells, thyroid cancer cells, squamous cell carcinoma cells, glioblastoma cells, neuroblastoma cells, uterine cancer cells, colorectal cancer cells, and pancreatic cancer cells.

In some embodiments, described herein is a method of inhibiting proliferation of tumor cells in a subject. For example, the method comprises administering an amount of a multispecific binding agent (e.g., an antibody) such as a multispecific binding agent that binds to CD47, including human CD47, and one or more targets that are not CD47 (e.g., PD-L1, including human PD-L1) described herein effective to inhibit proliferation of the tumor cells. In some embodiments, the method includes administering a multispecific binding agent (e.g., an antibody) that competes for binding with antibody mAb-C (see, e.g., CDRs and VH/VL of Table 1), to human CD47 and/or binds the region of a CD47 recognized by antibody mAb-C (see, e.g., CDRs and VH/VL of Table 1), resulting in resulting in enhancement of the removal of tumor cells. In some embodiments, the method includes administering a multispecific binding agent (e.g., an antibody) that competes for binding with antibody mAb-P (see, e.g., CDRs and VH/VL of Table 2), to human PD-L1 and/or binds the region of a PD-L1 recognized by antibody mAb-P (see, e.g., CDRs and VH/VL of Table 2), resulting in resulting in enhancement of the removal of tumor cells. In some embodiments, one or more polynucleotides, vectors, and/or cells as described above can be used in methods of enhancing the removal of tumor cells in vivo (e.g., in a method of treating cancer in a subject).

A method of modulating (e.g., inhibiting, reducing, preventing) tumor growth in a subject also is provided. For example, the method comprises administering to the subject a composition comprising a multispecific binding agent (e.g., an antibody) in an amount effective to modulate tumor growth in the subject.

As used herein, “tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth. Examples of cancers include, but are not limited to: breast cancer, colon cancer, renal cancer, lung cancer, squamous cell myeloid leukemia, hemangiomas, melanomas, astrocytomas, and glioblastomas as well as other cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia, renal cell carcinoma), bladder and urethra (squamous cell carcinoma, transitional cell carcinoma, adenocarcinoma), prostate (adenocarcinoma, sarcoma, small cell carcinoma of the prostate), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma) serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma; as well as cancers of the thyroid including medullary thyroid cancer.

In some embodiments, the tumor is a solid tumor. In some embodiments, the tumor or cancer is not a solid tumor. In further embodiments, the cancer is a leukemia cancer. In some embodiments, the tumor or cancer is a relapsed tumor or cancer. In some embodiments, the tumor or cancer is a metastatic tumor or cancer. In some embodiments, the tumor or cancer is a primary tumor or cancer. In some embodiments, the tumor or cancer reaches a remission, but can relapse or is at risk of relapsing. In some embodiments, the tumor or cancer is unresectable. Additionally or alternatively, the tumor or cancer is resistant to a chemotherapy or other anti-cancer therapy. In further embodiments, the cancer or tumor expresses CD47. Additionally or alternatively, the cancer or tumor expresses PD-L1.

In some embodiments, the disease or disorder is a cancer or a tumor. In some embodiments, the disease or disorder is a suppression of an immune response, for example, an immune response to a cancer or a tumor. In further embodiments (such as ADCC or ADCP), the cancer or tumor expresses CD47. Additionally or alternatively, the cancer or tumor expresses PD-L1. In some embodiments, the subject is a human subject.

Also provided is a method of treating cancer or tumor by administering a multispecific binding agent (e.g., an antibody) such as a multispecific binding agent that binds to CD47, including human CD47, and one or more targets that are not CD47 (e.g., PD-L1, including human PD-L1), to a subject in need thereof, alone or in combination with another agent. In some embodiments, provided herein is a use of a multispecific binding agent for treating a cancer or tumor. In further embodiments, the use comprises administering a multispecific binding agent as disclosed herein to a subject in need thereof, alone or in combination with another agent. In some embodiments, the subject in need has a cancer or a tumor. In further embodiments, the cancer or tumor expresses CD47. Additionally or alternatively, the cancer or tumor expresses PD-L1.

“Enhancing” tumor cell removal does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a multispecific binding agent (e.g., an antibody) of the method.

Additionally, multispecific binding agents (e.g., antibodies, such as bispecific antibodies) may be used to alleviate or reduce side effects associated with cancer, for example, bone deterioration, vertebral collapse, and paralysis. In one aspect, the subject suffers from or is at risk of suffering from bone metastases and a multispecific binding agent (e.g., an antibody) is administered in an amount to reduce deterioration of surrounding bone. Accordingly, in some aspects, a multispecific binding agent prevents bone deterioration due to bone metastases, wherein tumor cell proliferation is or is not reduced. In some aspects, a multispecific binding agent (e.g., an antibody) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

Additionally or alternatively, compared to a benchmark anti-CD47 antibody, multispecific binding agents (e.g., antibodies, such as bispecific antibodies) as disclosed herein alleviate or reduce a side effect. In some embodiments, the side effect is associated with inhibiting the interaction between CD47 and SIRPα. In some embodiments, the side effect is anemia and/or thrombocytopenia. Accordingly, in some embodiments, a multispecific binding agent as disclosed herein prevents or inhibits anemia and/or thrombocytopenia. In some embodiments, a multispecific binding agent (e.g., an antibody) further reduces tumor cell proliferation.

Also provided herein is a method for treating a tumor expressing CD47 in a subject without significantly inducing anemia and/or thrombocytopenia in the subject, comprising administering a multispecific binding agent as disclosed herein or a composition as disclosed herein. Additionally provided herein is a use of a multispecific binding agent as disclosed herein for treating a tumor expressing CD47 in a subject without significantly inducing anemia and/or thrombocytopenia. In some embodiments, the method or use does not induce anemia and/or thrombocytopenia in the subject. In some embodiments, the subject experiences anemia and/or thrombocytopenia, but the anemia and/or thrombocytopenia is significantly (for example, at least 10%, or at least 20%, or at least 30%, or at least 40%, or at least 50%, or at least 60%, or at least 70%, or al least 80%, or at least 90%, or at least 95%, or at least 99%) less than a control subject receiving another anti-CD47 therapy.

In some embodiments of any methods or uses as disclosed herein, a subject received the binding agent or composition as provided herein develops less side effects (such as a lower occurring frequency of a side effect, less kinds of side effects, and/or lower severity of a symptom) compared to another anti-CD47 therapy by about 10% to 90% (for example, by about 10%, by about 20%, by about 30%, by about 40%, by about 50%, by about 60%, by about 70%, by about 80%, by about 90%, or by about 95%, or by about 99%, or by about 100%). In further embodiments, the side effect is anemia and/or thrombocytopenia. In some embodiments of any methods or uses as disclosed herein, a subject received the binding agent or composition as provided herein is less likely to experience a side effect (such as anemia and thrombocytopenia) compared to another anti-CD47 therapy by about 10% to about 90% (for example, by about 10%, by about 20%, by about 30%, by about 40%, by about 50%, by about 60%, by about 70%, by about 80%, by about 90%, or by about 95%, or by about 99%, or by about 100%).

In some embodiments of any methods or uses as disclosed herein, the multispecific binding agent comprises an effector-competent Fc. As used herein, an effector-competent Fc refers to an Fc exhibiting one or more effector functions, such as participation of the antibody in antibody dependent cellular cytotoxicity (ADCC), ADCP and complement dependent cytotoxicity (CDC). In further embodiments, the multispecific binding agent lacks any one or any two or all three of the following mutations: L234A, L235A, and P329K (according to the EU numbering). In some embodiments, an effector-competent Fc lacks any one or any two or all three of the following mutations: L234A, L235A, and P329K (according to the EU numbering). In some embodiments, an effector-competent Fc comprises the following: L234, L235, and P329 (according to the EU numbering).

The subject of a method or use described herein can be administered one or more additional therapeutic agents in combination with a binding agent (e.g., a multispecific binding agent, such as a bispecific antibody) described herein or fragment thereof or a composition described herein. An additional agent can be an agent that targets a tumor or a cancer cell. Additionally or alternatively, an additional agent can also be an agent that targets an immune cell (e.g., an NK cell or a T cell).

In some embodiments, the binding agent or composition provided herein increases a therapeutic effect of the additional agent by about 10%-90% or about 2-100 folds. In some embodiments, the binding agent or composition provided herein increases a therapeutic effect of the additional agent by at least 10%. In some embodiments, the binding agent or composition provided herein increases a therapeutic effect of the additional agent by at least 20%. In some embodiments, the binding agent or composition provided herein increases a therapeutic effect of the additional agent by at least 30%. In some embodiments, the binding agent or composition provided herein increases a therapeutic effect of the additional agent by at least 40%. In some embodiments, the binding agent or composition provided herein increases a therapeutic effect of the additional agent by at least 50%. In some embodiments, the binding agent or composition provided herein increases a therapeutic effect of the additional agent by at least 60%. In some embodiments, the binding agent or composition provided herein increases a therapeutic effect of the additional agent by at least 70%. In some embodiments, the binding agent or composition provided herein increases a therapeutic effect of the additional agent by at least 80%. In some embodiments, the binding agent or composition provided herein increases a therapeutic effect of the additional agent by at least 90%. In some embodiments, the binding agent or composition provided herein increases a therapeutic effect of the additional agent by at least 2 fold. In some embodiments, the binding agent or composition provided herein increases a therapeutic effect of the additional agent by at least 5 fold. In some embodiments, the binding agent or composition provided herein increases a therapeutic effect of the additional agent by at least 10 fold. In some embodiments, the binding agent or composition provided herein increases a therapeutic effect of the additional agent by at least 20 fold. In some embodiments, the binding agent or composition provided herein increases a therapeutic effect of the additional agent by more than 50 fold.

A particular administration regimen of a multispecific binding agent (e.g., an antibody) for a particular subject will depend, in part, upon the agent used, the amount of agent administered, the route of administration, and the cause and extent of any side effects. The amount of agent (e.g., an antibody) administered to a subject (e.g., a mammal, such as a human) should be sufficient to effect the desired response over a reasonable time frame. Accordingly, in some embodiments, the amount of a multispecific binding agent (e.g., an antibody) or pharmaceutical composition described herein administered to a subject is an effective amount. In some embodiments, the amount of a multispecific binding agent (e.g., an antibody) or pharmaceutical composition described herein administered to a subject is a therapeutically effective amount. In some aspects, the method comprises administering, e.g., from about 0.1 μg/kg to up to about 100 mg/kg or more. In some embodiments, the dosage ranges from about 1 μg/kg up to about 100 mg/kg; or about 5 μg/kg up to about 100 mg/kg; or about 10 μg/kg up to about 100 mg/kg; or about 1 mg/kg up to about 50 mg/kg; or about 2 mg/kg up to about 30 mg/kg; or about 3 mg/kg up to about 25 mg/kg; or about 3 mg/kg up to about 25 mg/kg; or about 5 mg/kg up to about 10 mg/kg; or about 10 mg/kg up to about 20 mg/kg; or about 10 mg/kg up to about 30 mg/kg. Some conditions or disease states require prolonged treatment, which may or may not entail administering doses of multispecific binding agents (e.g., antibodies, such as bispecific antibodies), including multispecific binding agents that bind to CD47, including human CD47, and one or more targets that are not CD47 (e.g., PD-L1, including human PD-L1) (e.g., antibodies, such as bispecific antibodies), over multiple administrations (e.g., every day, three times a week, once a week, once every two weeks, or once every month for a treatment period of three days, seven days, two weeks, three weeks, one month, three months, six months, nine months, 12 months, 15 months, 18 months, 21 months, two years, or more).

Suitable routes of administering a composition comprising a multispecific binding agent (e.g., an antibody), such as a multispecific binding agent that binds to CD47, including human CD47, and one or more targets that are not CD47 (e.g., PD-L1, including human PD-L1) (e.g., an antibody), are well known in the art. Although more than one route can be used to administer an agent (e.g., an antibody), a particular route can provide a more immediate and more effective reaction than another route. Depending on the circumstances, a composition comprising a multispecific binding agent (e.g., an antibody) such as a multispecific binding agent that binds to CD47, including human CD47, and one or more targets that are not CD47 (e.g., PD-L1, including human PD-L1) is applied or instilled into body cavities, absorbed through the skin or mucous membranes, ingested, inhaled, and/or introduced into circulation. For example, it may be desirable to deliver a composition comprising a multispecific binding agent (e.g., an antibody) such as a multispecific binding agent that binds to CD47, including human CD47, and one or more targets that are not CD47 (e.g., PD-L1, including human PD-L1) through injection by intravenous, subcutaneous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, a multispecific binding agent (e.g., an antibody) such as a multispecific binding agent that binds to CD47, including human CD47, and one or more targets that are not CD47 (e.g., PD-L1, including human PD-L1) is administered regionally via intraarterial or intravenous administration feeding the region of interest, e.g., via the hepatic artery for delivery to the liver. Alternatively, a multispecific binding agent (e.g., an antibody) such as a multispecific binding agent that binds to CD47, including human CD47, and one or more targets that are not CD47 (e.g., PD-L1, including human PD-L1) is administered locally via implantation of a membrane, sponge, or another appropriate material on to which the binding agent has been absorbed or encapsulated. Where an implantation device is used, the device is, in one aspect, implanted into any suitable tissue or organ, and delivery of a multispecific binding agent (e.g., an antibody) such as a multispecific binding agent that binds to CD47, including human CD47, and one or more targets that are not CD47 (e.g., PD-L1, including human PD-L1) is, for example, via diffusion, timed-release bolus, or continuous administration. In other aspects, the multispecific binding agent is administered directly to exposed tissue during tumor resection or other surgical procedures.

### Compositions and Combination Therapies

The present disclosure provides a composition, such as pharmaceutical composition, comprising a multispecific binding agent (e.g., an antibody) such as a multispecific binding agent that binds to CD47, including human CD47, and one or more targets that are not CD47 (e.g., PD-L1, including human PD-L1) and a carrier (e.g., a pharmaceutically acceptable carrier). The particular carrier employed may depend on chemico-physical considerations, such as solubility and lack of reactivity with the binding agent or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). A pharmaceutical composition comprising a multispecific binding agent (e.g., an antibody) such as a multispecific binding agent that binds to CD47, including human CD47, and one or more targets that are not CD47 (e.g., PD-L1, including human PD-L1) is, in one aspect, placed within containers, along with packaging material that provides instructions regarding the use of such pharmaceutical compositions. Generally, such instructions include a tangible expression describing the reagent concentration, as well as, in some embodiments, relative amounts of excipient ingredients or diluents (e.g., water, saline or PBS) that may be necessary to reconstitute the pharmaceutical composition.

In one aspect, the present disclosure further provides a composition, such as a pharmaceutical composition, comprising at least one of the following: a binding agent provided herein (e.g., a multispecific binding agent, or one antibody or antigen-binding fragment thereof of the present disclosure), a nucleic acid or a polynucleotide provided herein, a vector provided herein, or a cell provided herein. In further embodiments, the composition further comprises a carrier (for example, a pharmaceutically acceptable carrier). In some embodiments, a pharmaceutical composition comprises a therapeutically effective amount of a binding agent provided herein (e.g., a multispecific binding agent or a bispecific antibody or antigen-binding fragment thereof provided herein) and a pharmaceutically acceptable carrier. In some embodiments, a pharmaceutical composition comprises a therapeutically effective amount of a nucleic acid provided herein (such as a nucleic acid encoding a bispecific antibody or antigen-binding fragment thereof provided herein) and a pharmaceutically acceptable carrier. In some embodiments, a pharmaceutical composition comprises a therapeutically effective amount of a vector provided herein (such as a vector comprising a nucleic acid as disclosed herein and expressing a binding agent as disclosed herein) and a pharmaceutically acceptable carrier. In some embodiments, a pharmaceutical composition comprises a therapeutically effective amount of a cell provided herein (such as a cell comprising a nucleic acid encoding a bispecific antibody or antigen-binding fragment thereof provided herein and/or expressing a bispecific antibody or antigen-binding fragment thereof provided herein) and a pharmaceutically acceptable carrier.

In some aspects of any method as disclosed herein, a composition comprising a binding agent as disclosed herein is administered. In some aspects, a method described herein further comprises administering one or more additional agents, including therapeutic agents (e.g., combination therapy), which may be present in a composition or may be administered with a multispecific binding agent (e.g., an antibody) such as a multispecific binding agent that binds to CD47, including human CD47, and one or more targets that are not CD47 (e.g., PD-L1, including human PD-L1) or provided in a separate composition using the same or a different route of administration. The one or more additional agents, including therapeutic agents, may be administered together or separately (e.g., simultaneously, alternatively, sequentially) with a multispecific binding agent (e.g., an antibody). Such additional therapeutic agents include, but are not limited to, therapeutic antibodies, immunotherapies, cytotoxic agents, chemotherapeutic agents, and inhibitors.

Therapeutic antibodies that can be used include, but are not limited to, trastuzumab; abciximab; daclizumab; BEC2; IMC-C22; vitaxin; Campath 1H/LDP-03; Smart M195; epratuzumab; bectumomab; visilizumab; CM3, a humanized anti-ICAM3 antibody; IDEC-I 14; ibritumomab tiuxetan; IDEC-131; IDEC-151; IDEC-152; SMART anti-CD3; eculizumab; adalimumab; certolizumab; IDEC-I 51; MDX-CD4; CD20-sreptdavidin; CDP571; LDP-02; OrthoClone OKT4A; ruplizumab; natalizumab; and lerdelimumab.

Immunotherapies that can be used include, but are not limited to, cytokines, such as granulocyte-macrophage colony-stimulating factor (GM-CSF), granulocyte-colony stimulating factor (G-CSF), macrophage inflammatory protein (MIP)-I-alpha, interleukins (including IL-I, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IF A); QS-21; DETOX; Levamisole; and Dinitrophenyl (DNP), and combinations thereof, such as, for example, combinations of, interleukins, for example, IL-2 with other cytokines, such as IFN-alpha. In some embodiments, the immunotherapy includes an immunotherapeutic agent that modulates immune responses, for example, a checkpoint inhibitor or a checkpoint agonist. In some embodiments, the immunotherapeutic agent is an antibody modulator that targets PD-1, PD-L1, PD-L2, CEACAM (e g., CEACAM-I, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity, inhibits suppression of an immune response, inhibits suppressor cells or suppressor cell activity, inhibits Treg activity, or inhibits the activity of inhibitory immune checkpoint receptors.

In some embodiments, the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-I, LFA-I (CD1 la/CDI8), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

Cytotoxic agents that can be used include a substance that inhibits or prevents a cellular function and/or causes cell death or destruction. Exemplary cytotoxic agents include, but are not limited to, radioactive isotopes (e.g., At211, 1131, 1125, Y90, Re186, Re188, Sm153, Bi212, P32, Pb212 and radioactive isotopes of Lu); growth inhibitory agents; enzymes and fragments thereof such as nucleolytic enzymes; and toxins such as small molecule toxins or enzymatically active toxins of bacterial, fungal, plant or animal origin, including fragments and/or variants thereof. Other exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents that can be used include chemical compounds useful in the treatment of cancer. Examples of chemotherapeutic agents include, but are not limited to, erlotinib, bortezomib, disulfiram, epigallocatechin gallate, salinosporamide A, carfilzomib, 17-AAG (geldanamycin), radicicol, lactate dehydrogenase A (LDH-A), fulvestrant, sunitib, letrozole, imatinib mesylate, fmasunate, oxaliplatin, 5-FET (5-fluorouracil), leucovorin, Rapamycin, Lapatinib, Lonafamib (SCH 66336), sorafenib, Bayer Labs), gefitinib, AG1478; alkylating agents such as thiotepa and CYTOXAN®; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; cally statin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5 alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancrati statin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omega I (Angew Chem. Inti. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Ore.); razoxane; rhizoxin; sizofuran; spirogermanium; tenuazonic acid; triaziquone; 2,2′,2″-trichlorotriethylamine; trichothecenes especially T-2 toxin, verracurin A, roridin A and anguidine); urethan; vindesine; dacarbazine; mannomustine; mitobronitol; mitolactol; pipobroman; gacytosine; arabinoside “Ara-C”); cyclophosphamide; thiotepa; taxoids, e.g., paclitaxel, ABRAXANE® (Cremophor-free), albumin-engineered nanoparticle formulations of paclitaxel (American Pharmaceutical Partners, Schaumberg, III.), and docetaxel/doxetaxel; chloranmbucil; gemcitabine; 6-thioguanine; mercaptopurine; methotrexate; platinum analogs such as cisplatin and carboplatin; vinblastine; etoposide (VP-16); ifosfamide; mitoxantrone; vincristine; vinorelbine; novantrone; teniposide; edatrexate; daunomycin; aminopterin; capecitabine; ibandronate; CPT-I I; topoisomerase inhibitor RFS 2000; difluorom ethyl ornithine (DMFO); retinoids such as retinoic acid; and pharmaceutically acceptable salts, acids and derivatives of any of the above. Chemotherapeutic agent also includes (i) anti-hormonal agents that act to regulate or inhibit hormone action on tumors such as anti-estrogens and selective estrogen receptor modulators (SERMs), including, for example, tamoxifen (including tamoxifen citrate), raloxifene, droloxifene, iodoxyfene, 4-hydroxytamoxifen, trioxifene, keoxifene, LY117018, onapristone, and toremifme citrate; (ii) aromatase inhibitors that inhibit the enzyme aromatase, which regulates estrogen production in the adrenal glands, such as, for example, 4 (5)-imidazoles, aminoglutethimide, megestrol acetate, exemestane, formestanie, fadrozole, vorozole, letrozole, and anastrozole; (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LEIRTOTECAN®; ABARELIX®; and (ix) pharmaceutically acceptable salts, acids and derivatives of any of the above.

Chemotherapeutic agents also include antibodies, as described above, including alemtuzumab, bevacizumab; cetuximab; panitumumab, rituximab, pertuzumab, tositumomab, and the antibody drug conjugate, gemtuzumab ozogamicin. Additional humanized monoclonal antibodies with therapeutic potential as agents in combination with the multispecific binding agents (e.g., antibodies) as described herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nivolumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-I2 (ABT-8744695, Wyeth Research and Abbott Laboratories) which is a recombinant exclusively human-sequence, full-length IgGI λ antibody genetically modified to recognize interleukin-12 p40 protein. Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-I 7-butyrate, clobetasol-1 7-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF alpha) blockers such as etanercept, infliximab, adalimumab, certolizumab pegol, golimumab (Simponi), Interleukin 1 (IL-I) blockers such as anakinra, T cell costimulation blockers such as abatacept, Interleukin 6 (IL-6) blockers such as tocilizumab; Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-MI prime; Secreted homotrimeric LTa3 and membrane bound heterotrimer LTal/132 blockers such as Anti-lymphotoxin alpha (LTa); miscellaneous investigational agents such as thioplatin, PS-341, phenylbutyrate, ET-18—OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol); beta-lapachone; lapachol; colchicines; betulinic acid; acetylcamptothecin, scopolectin, and 9-aminocamptothecin); podophyllotoxin; tegafur; bexarotene; bisphosphonates such as clodronate, etidronate, NE-58095, zoledronic acid/zoledronate, alendronate, pamidronate, tiludronate, or risedronate; and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (R11577); orafenib, ABT510; Bcl-2 inhibitor such as oblimersen sodium pixantrone; farnesyltransferase inhibitors such as lonafamib (SCH 6636); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin combined with 5-FU and leucovorin. Chemotherapeutic agents also include Poly ADP ribose polymerase (PARP) inhibitors: olaparib, rucaprib niraparib, talzoparib.

Inhibitors that can be used include, but are not limited to, kinase inhibitors such as imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilter, larotrectinib, lorlatinib, netarsudil, osimertinib, pemigatinib, pexidartinib, ribociclib, temsirolimus, XL-092, XL-147, XL-765, XL-499, and XL-880. In some embodiments, a compound as described herein can be used in combination with a HSP90 inhibitor (e.g., XL888), liver X receptor (LXR) modulators, retinoid-related orphan receptor gamma (RORy) modulators, checkpoint inhibitors such as a CK1 inhibitor or aCK1a inhibitor, a Wnt pathway inhibitor (e.g., SST-215), or a mineralocorticoid receptor inhibitor, (e.g., esaxerenone) or XL-888 for the treatment of a disease disclosed herein such as cancer. In some embodiments, a multispecific binding agent (e.g., an antibody) as disclosed herein can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, 1NS-R, IGF-1R, IR-R, PDGFαR, PDGFβ/R, CSFIR, KIT, FLK-II, KDR/FLK-1, FLK-4, flt-1, FGFR1, FGFR2, FGFR3, FGFR4, Ron, Sea, TRKA, TRKB, TRKC, FLT3, VEGFR/FIt2, Flt4, EphAl, EphA2, EphA3, EphB2, EphB4, Tie2, Src, Fyn, Lck, Fgr, Btk, Fak, SYR, FRK, JAK (JAK1 and or JAK2), ABL, ALK, CDK7, CDK12, KRAS, and B-Raf.

Additional non-limiting examples of inhibitors that can be combined with a multispecific binding agent (e.g., an antibody) of the present disclosure for treatment of cancer include an FGFR inhibitor (FGFR1, FGFR2, FGFR3 or FGFR4, e.g., pemigatinib, an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, vandetanib, ramucirumab, lenvatinib, ziv-aflibercept), a PARP inhibitor (e.g. olaparib, rucaparib, veliparib or niraparib), a JAK inhibitor (e.g., ruxolitinib, baricitinib, itacitinib), an IDO inhibitor (e.g., epacadostat, NLG919, or BMS-986205, MK7162), an LSD1 inhibitor, a TDO inhibitor, a PI3K-delta inhibitor (e.g., parsaclisib), a PI3K-gamma inhibitor such as PI3K-gamma selective inhibitor, a Pim inhibitor, a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors, or combinations thereof.

In some embodiments, a multispecific binding agent as disclosed herein can be used in combination with inhibitors of PD-1 or inhibitors of PD-L1, e.g., an anti-PD-1 monoclonal antibody or an anti-PD-L1 monoclonal antibody, for example, nivolumab (Opdivo), pembrolizumab (Keytruda, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZM009, AK105, HLX10, and TSR-042, AMP-224, AMP-514, PDR001, durvalumab, pidilizumab (Imfinzi®, CT-011), CK-301, BMS 936559, MPDL3280A, tislelizumab, BMS-935559, MEDI4736, FAZ053, KN035, CS1001, CBT-502, A167, STI-A101, BGB-A333, MSB-2311, HLX20, AUNP12, CA-170, BMS-986189, LY3300054, and MSB0010718C.

In some embodiments, a multispecific binding agent as disclosed herein can be used in combination with CTLA-4 inhibitors, e.g., an anti-CTLA-4 antibody, for example, ipilimumab (Yervoy), tremelimumab and AGEN1884, or with phosphatidylserine inhibitors, for example, bavituximab (PGN401), or with antibodies to cytokines (IL-10, TGF-b, and the like), or with bispecific antibodies that bind to PD-L1 and CTLA-4 (e.g., AK104) or PD-1 and CTLA-4, or with other anti-cancer agents such as cemiplimab.

The additional agent may be a pharmaceutically acceptable salt, ester, amide, hydrate, and/or prodrug of any of these therapeutic agents described above or other agents.

### Diagnostic & Detection

Labeled binding molecules such as labeled antibodies and derivatives and analogs thereof which immunospecifically bind to an antigen (including mAb-C, a binding agent comprising the CDRs of mAb-C, a binding agent comprising the variable regions of mAb-C, a binding agent binding to CD47 competitively with mAb-C, mAb-P, a binding agent comprising the CDRs of mAb-P, a binding agent comprising the variable regions of mAb-P, a binding agent binding to PD-L1 competitively with mAb-P, or a multispecific binding agent as disclosed herein) can be used for diagnostic purposes to detect, diagnose, or monitor a disease, and/or for selecting a subject suitable for a method or a use as disclosed herein. In some embodiments, the use and/or method comprises contacting the labeled binding molecule with a biological sample obtained from a subject (such as a subject suspect of having the disease), and detecting a complex comprising the labeled binding molecule and any one or more of the following: CD47, PD-L1, or a moiety (such as a cell) expressing either or both of CD47 and PD-L1. In some embodiments, the use and/or method comprises administering the labeled binding molecule to a subject, such as a subject suspect of having the disease, and detecting a complex comprising the labeled binding molecule and any one or mor of the following: CD47, PD-L1, or a moiety (such as a cell) expressing either or both of CD47 and PD-L1.

Antibodies provided herein (including mAb-C, a binding agent comprising the CDRs of mAb-C, a binding agent comprising the variable regions of mAb-C, a binding agent binding to CD47 competitively with mAb-C, mAb-P, a binding agent comprising the CDRs of mAb-P, a binding agent comprising the variable regions of mAb-P, a binding agent binding to PD-L1 competitively with mAb-P, or a multispecific binding agent as disclosed herein) can be used to assay an antigen level in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### Kits

Also provided herein are kits comprising a binding agent (e.g., a multispecific agent such as a bispecific antibody) provided herein, or a composition (e.g., a pharmaceutical composition) provided herein, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

It is understood that modifications which do not substantially affect the activity of the various embodiments described herein are also provided within the definition of the subject matter described herein. Accordingly, the following examples are intended to illustrate but not limit the present disclosure.

### EXAMPLES

Programmed death ligand-1 (PD-L1) and CD47 are two targets widely expressed on the cell surface of tumor cells and have been shown to coordinately suppress adaptive and innate sensing, respectively, to evade immune control. PD-L1 dampens T cell-mediated tumor killing (via PD-L1/PD-1 signaling), while CD47 protects tumor cells from phagocytosis (via CD47/SIRP-alpha signaling). Monoclonal antibody-based therapies targeting PD-L1 and PD-1 have shown promise, but a large number of patients remain unresponsive to treatment. Immunotherapies targeting the CD47 pathway have shown clinical efficacy in a variety of tumors, but have been associated with adverse effects, such as anemia and thrombocytopenia, as well as reduced bioavailability, due to CD47 expression on peripheral blood cells.

To improve selectivity and therapeutic index, generated herein are antibodies as disclosed herein (such as bsAb1), which are affinity-tuned bispecific that target PD-L1 and CD47 with high fidelity. bsAb1 was generated and engineered specially with a high affinity binding PD-L1 arm and a detuned CD47 arm, enabling tight binding to tumor cells while minimizing binding to red blood cells (RBCs). bsAb1 also incorporated an effector-competent Fc domain to enhance the induction of phagocytosis and other effector functions.

The affinity-tuned bsAb1 demonstrated significant blockade of PD-L1/PD-1 and SIRPα/CD47 signaling in vitro, as well as tumor growth inhibition in animal models. bsAb1 also showed minimal binding to human RBCs and no hemagglutination activity. In addition, bsAb1 was able to induce strong phagocytotic activity mediated by human macrophages against human tumor cell line expressing PD-L1 and CD47. Importantly, bsAb1 was found to be well-tolerated in non-human primates.

Thus, bsAb1, an affinity-tuned bispecific antibody targeting PD-L1 and CD47, two key negative immune checkpoints of the innate and adaptive response, has a favorable activity and tolerability profile that supports its further development. More details are described below.

### Example 1. Bispecific Antibody: Generation and Binding Specificity

Antibodies to CD47 or PD-L1 were generated by phage display and bispecific antibodies were constructed from the selected antibodies.

To obtain binders for human CD47 and PD-L1, antibody discovery was conducted by phage display of human Fab. The extracellular domain of human CD47 was purchased from Acro Biosystems (biotinylated human CD47 His-Avitag Acro Cat No. CD7-H82E9 and human CD47 His-tag Acro Cat No. CD7-5227). The extracellular domain of human PD-L1 was purchased from Acro Biosystems (human PD-L1-His tag Acro Cat. No. PD1H5229, biotinylated human PD-L1-His Avitag Acro Cat. No. PDL-H82E4). The non-biotinylated extracellular domain of CD47 or PD-L1 was biotinylated using EZ-Link NHS-PEG12-Biotin (ThermoScientific Cat. No. 21312) using standard protocol as needed. Phage clones were screened for the ability to bind to biotinylated human CD47 or biotinylated human PD-L1 by phage ELISA using standard protocols. Briefly, Fab-formatted phage libraries were constructed using expression vectors capable of replication and expression in phage (also referred to as a phagemid). Both the heavy chain and the light chain were encoded in the same expression vector, where the heavy chain was fused to a truncated variant of the phage coat protein plll. The light chain and heavy chain-plll fusion were expressed as separate polypeptides and assembled in the bacterial periplasm, where the redox potential enables disulfide bond formation, to form the antigen binding domain (Fab) of the candidate antibody.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:68) and light chain (SEQ ID NO: 69) scaffolds used in the library are listed below, where a lower case “x” represents CDR amino acids that were varied to create the library, and bold italic represents the CDR sequences that were constant.

The sequence for SEQ ID NO: 68 was
EVQLVESGGGLVQPGGSLRLSCAASGFTFSXXXXXWVRQAPGKGL
EWVAXXXXXXXXXXXXXXXXXRFTISADTSKNTAYLQMNSLRAED
TAVYYCARXXXXXXXXXXXXXXWGQGTLVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
C.
The sequence for SEQ ID NO: 69 was
DIQMTQSPSSLSASVGDRVTITCRASQSVSSAVAWYQQKPGKAPK
LLIYSASSLYSGVPSRFSGSRSGTDFTLTISSLQPEDFATYYCXX
XXXXXXXFGQGTKVEIKRTVAAPSVFIFPPSDSQLKSGTASVVCL
LNNFYPREAKVQWKVDNALQSGNSQESVTEQDSKDSTYSLSSTLT
LSKADYEKHKVYACEVTHQGLSSPVTKSFNRGEC.

Diversity was created through mutagenesis using degenerate DNA oligonucleotide primers to introduce diversity into VL CDR3 and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety. Briefly, uracil-incorporated single-stranded circular DNA were prepared from isolated phage using standard procedures and Kunkel mutagenesis carried out to introduce diversity to the four CDRs. Chemically-synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to approximately 1×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀ of approximately 0.6. After 20 minutes incubation at room temperature, infected cells were sub-cultured in 25 mL 2×YT+Ampicillin and M13K07 helper phage (final concentration of approximately 1×10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with bead-immobilized CD47 or PD-L1 target antigen using standard procedures (round 3:100 nM CD-47 and 50 nM PD-L1, round 4:50 nM CD-47 and 25 nM PD-L1). In total, 3-4 rounds of phage panning were performed to enrich in phage displaying Fabs specific for the target antigen. Target-specific enrichment was confirmed using polyclonal ELISA and individual clones were isolated and further verified by performing monoclonal phage ELISA. DNA sequencing was used to determine the sequence of the CDRs of isolated Fab clones containing a candidate antibody.

To measure binding affinity in CD47 or PD-L1 binder discovery campaigns, the VL and VH domains identified in the phage screen described above were formatted into a bivalent monospecific native human full-length IgG1 architecture and immobilized to a biosensor on an Octet (Pall ForteBio) biolayer interferometer. Soluble antigens (e.g., CD47 or PD-L1) were then added to the system and binding was measured.

Antibodies were affinity purified using anti-IgG10-CH1 resign by batch-mode gravity filtration. Clarified supernatants generated from 500 mL or 1000 mL transfection volumes were affinity purified using CaptureSelect™ CH1-XLAffinity Matrix bulk resin (ThermoFisher PN 194346201L). Supernatants from 1000 mL transfections were divided into 500 mL portions and all supernatants were transferred to 1 L sterile shake flasks. Resin was washed free of storage buffer using Dulbecco's PBS (pH 7.4, without Ca2+/Mg2+) by gravity filtration using disposable 10 mL columns (ThermoFisher Scientific PN 29924) and resuspended in PBS as a 50% slurry. Resin was aliquoted to shake flasks on a scale of 6 mL of 50% resin slurry per 500 mL of supernatant. Supernatant was incubated with resin for 1-2 hours at room temperature on a flask shaker operated at 170 RPM. Resin was captured on a gravity filtration column equipped with reservoir attachment (GE Healthcare Life Sciences PN 18-3216-03) and washed with 30 mL DPBS (10× settled resin bed volume). The resin bed was stringently washed with 30 mL with DPBS containing 500 mM NaCl. An additional wash was performed using 30 mL DPBS to reduce the concentration of NaCl prior to elution. Bound antibody was eluted from the gravity column using 9 mL 0.1 M sodium acetate (pH 3.5) into 2.25 mL neutralization buffer (2 M Tris-HCl, pH 7.5; Sigma PN T2944) contained in a 50 ml conical tube. Samples in neutralized elution buffer were dialyzed into DPBS overnight at 4° C. using a dialysis cassette with a 3.5 kDa molecular weight cutoff (Slide-A-Lyzer; ThermoFisher Scientific, PN 66110).

A bispecific antibody was constructed from a selected CD47 binding antibody (mAb-C) with CDR, VH, and VL sequences as shown in Table 1 and a selected and then affinity matured PD-L1 binding antibody (mAb-P) with CDR, VH, and VL sequences as shown in Table 2. This selected bispecific antibody in an exemplary format as a four polypeptide chain antibody with a first polypeptide chain having an amino acid sequence of SEQ ID NO:48, a second polypeptide chain having an amino acid sequence of SEQ ID NO:49, a third polypeptide chain having an amino acid sequence of SEQ ID NO:50, and a fourth polypeptide chain having an amino acid sequence of SEQ ID NO:51 (designated herein as bsAb1) was affinity purified and each affinity-purified preparation was polished by strong cation exchange (SCX) using an AKTA pure fast protein liquid chromatography instrument (FPLC, GE Healthcare Life Sciences) running GE Unicorn v7.2 software. Aseptic techniques were used at all times. Prior to the day of purification, the entire flow-path (including the mobile phase reservoirs, samples loop, column, and switching valves) was cleaned using 500 mM NaOH in 20% ethanol by slowly pumping the cleaning solution through the instrument (0.1 mL/min for 2 hours). The instrument was then flushed with 20% ethanol and stored overnight. Just prior to polishing, a sample was buffer exchanged into 20 mM MES buffer [pH 6.0, MES=(2-(Nmorpholino) ethanesulfonic acid]. A buffer exchange method was employed using molecular weight cutoff (MWCO) filters with buffer exchange performed within 1-2 hours of polishing with storage at room temperature until polishing commenced. Approximately 35 mg of antibody (˜ 4 mg/mL) in DPBS was added to a 15 mL, 30 kDa MWCO centrifugal spin filter (Amicon Ultra 30) and brought up to volume with MES buffer. Sample was centrifuged at 4k RPM for 15 minutes or until the original sample volume was achieved. MES buffer was added to bring the volume up to 15 mL and the sample was centrifuged again. This process was repeated three more times. 10 mL of low-endotoxin sample representing ˜35 mg of protein at 3.5 mg/ml was loaded onto a MonoS 5/50 GL SCX column (0.5 cm×5 cm, 1 mL, 10 μm particle diameter; GE Healthcare Life Sciences, PN17516801) at 0.5 mL/min in mobile phase A (20 mM MES, pH 6.0) using 12 mL of mobile phase A. An additional 2 CV (column volume) of mobile phase A was used to remove unbound sample at a flow rate of 2.0 mL/min. 1.0 CV was 0.982 mL, as defined in GE Unicorn software for this particular column. Sample was eluted at 2.0 mL/min using a linear gradient from 0 to 30% mobile phase B (20 mM MES/1 M NaCl, pH 6.0) over 50 CV with fractions collected at 1-mL intervals. The system was washed with 7 CV of 100% B at 2.0 mL/min. Fractions from each major peak were pooled and assessed by non-reducing SDS-PAGE. The pooled sample was buffer exchanged into 1×DPBS as described in the procedure above, sterile filtered through a 0.2 μm filter, and re-tested for endotoxin using the ToxinSensor Chromogenic LAL Endotoxin Assay Kit from GenScript (PN L00350).

This process was repeated several times until unpolished input material was exhausted. A master pool was created from purified material from each injection and was subjected to full analytics screening and freeze/thaw analysis.

The bispecific antibody selected and purified as described above (designated bsAb1 herein) was tested for binding. Carterra was used to confirm the specific interaction of the antigens to the selected bispecific antibody and to determine the antigen binding affinities. To monitor the interaction with the antigen, the selected bispecific antibody was immobilized on a biosensor, and its interaction with soluble antigen was measured using a Carterra instrument. First, the selected bispecific antibody was covalently linked to a Carterra HC30M chip. To obtain accurate kinetic constants, the bispecific antibody was prepared at concentrations ranging from 1 to 10 μg/mL in 10 mM NaOAc (pH 4.25 or pH 4.5), and the coupling reaction was performed in running buffer (25 mM MES, pH 5.5) using multiple chip locations for each concentration. In the next association step, the binding interaction of antigen to the coupled antibody was measured. In general, each binding event consisted of a one minute baseline interval followed by a 5 minute antigen injection and a 15 minute dissociation with HBSTE buffer (10 mM HEPES, 150 mM NaCl, 3 mM EDTA, 0.01% Tween-20, pH 7.4) as running buffer. Human and cynomolgus orthologs for both CD47 and PD-L1/B7-H1 were obtained in proteins of His-tagged format from AcroBiosystems (Human PD-L1 (Cat #PD1-H82E5), Human CD47 (Cat #CD7-H82E9), Cyno PD-L1 (Cat #PD1-C52H4), Cyno CD47 (Cat #CD7-C52H1)). Antigens for binding studies were then prepared by reconstituting according to manufacturer recommendation. To obtain accurate kinetic constants, the reconstituted antigens were subsequently diluted into HBSTE buffer to final concentrations ranging from 1.2 nM to 900 nM. Injections of the antigens at each concentration were performed in series. In the next dissociation step, a regeneration process consisting of three times of injection for 25 seconds with 0.1 M glycine (pH 2.8), 1 M NaCl was performed between each antigen binding event, by which the bound antigen on the surface of the chip was allowed to dissociate. Carterra Kinetics analysis package was used to calculate rate constants (kₐ, k_{d}) and corresponding equilibrium dissociation constants (Kd). Binding data were first recovered by double-referencing to subtract the closest reference cell and blank injection. The analysis was then performed by globally fitting the association and dissociation profiles of each sensorgram. A minimum of 30 chip locations that displayed sufficiently high signal-to-noise ratio were averaged to determine mean and standard deviations for each parameter of an antibody-antigen pair.

Exemplary results for Carterra analysis are shown in Table 3A. Results indicate that the affinity of the selected bispecific antibody against human CD47 is similar to its affinity against cynomolgus CD47. For PD-L1 antigen, the selected bispecific antibody also shows a similar affinity against PD-L1 from human and cynomolgus.

TABLE 3A
Carterra affinity analysis

| | | | Kd (nM) |
|---|---|---|---|
| Antigen | kₐ (M⁻¹s⁻¹) | k_{d} (s⁻¹) | (via kinetics) |
| Human CD47 | 3.5 (±0.3) × 10⁵ | 9.3 (±0.5) × 10⁻³ | 27 (±4) |
| Cynomolgus | 1.9 (±0.3) × 10⁵ | 4.6 (±0.9) × 10⁻³ | 24 (±4) |
| CD47 | | | |
| Human PD-L1 | 9.8 (±1.6) × 10⁵ | 1.3 (±0.4) × 10⁻⁴ | 0.15 (±0.6) |
| Cynomolgus | 8.9 (±1.3) × 10⁵ | 1.5 (±0.7) × 10⁻⁴ | 0.18 (±0.10) |
| PD-L1 | | | |

TABLE 3B
Biacore affinity analysis

| Antigen | ka (1/M, s) | kd (1/s) | KD (nM) |
|---|---|---|---|
| human-PD-L1 | 3.30 × 10⁶ | 1.64 × 10⁻⁴ | 0.050 |
| cyno-PD-L1 | 3.54 × 10⁶ | 1.20 × 10⁻⁴ | 0.034 |
| human-CD47 | 6.72 × 10⁵ | 1.41 × 10⁻² | 21.0 |
| cyno-CD47 | 5.46 × 10⁵ | 1.37 × 10⁻² | 25.0 |

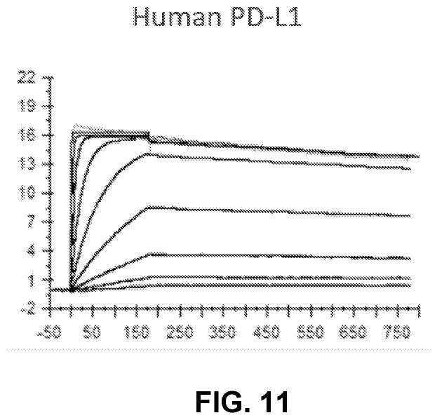

Further, the affinity of the selected bispecific antibody to human PD-L1, cynomolgus PD-L1, human CD47 and cynomolgus CD47 was measured using Biacore. An exemplary Biacore sensorgram is provided in FIG. 11, while the calculated affinity data is provided in Table 3B.

### Example 2. Bispecific Antibody: Cell Binding

The selected bispecific antibody in an exemplary format (designated bsAb1 herein) was evaluated for binding to cells expressing CD47 and PD-L1, for example, PDL1-MDA-MB-231 cells.

Cells were harvested at 70-90% confluence on the day of the assay. Cells were collected by centrifugation at 200-300×g for 5 minutes and media was removed. Cells were resuspended at 2×10⁶ cells per mL in cold PBS. An 8-point antibody dilution series (2× concentration) was prepared in PBS to cover the expected binding affinities of the antibodies being tested. 50 μL per well of the antibody dilution was plated in a 96-well V-bottom plate (Costar 3897). 50 μL per well of cell suspension was added. Plates were placed at 4° C. for 45-60 minutes. 100 μL per well PBS was added.

Cells were collected by centrifugation at 400×g for 5-7 minutes and primary antibody was removed. 50 μL per well of Alexa Fluor 488 goat anti human IgG Fab (Jackson Immuno Research 109-547-003) at 1:100 dilution was added. Plates were placed at 4° C. for 30 minutes.

Cells were collected by centrifugation at 400×g for 5-7 minutes and secondary antibody was removed. Cells were resuspended in 50 μL per well of PBS and analyzed by flow cytometry. Binding curves were calculated using the mean fluorescence intensity (MFI) of the Alexa Fluor 488 fluorescence signal on the cells.

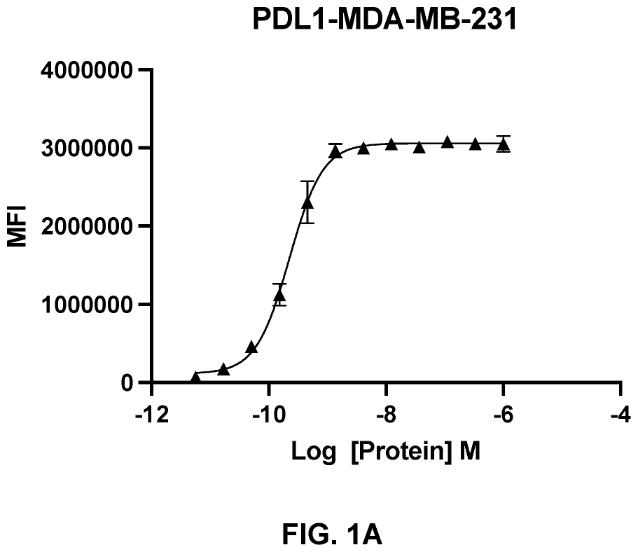

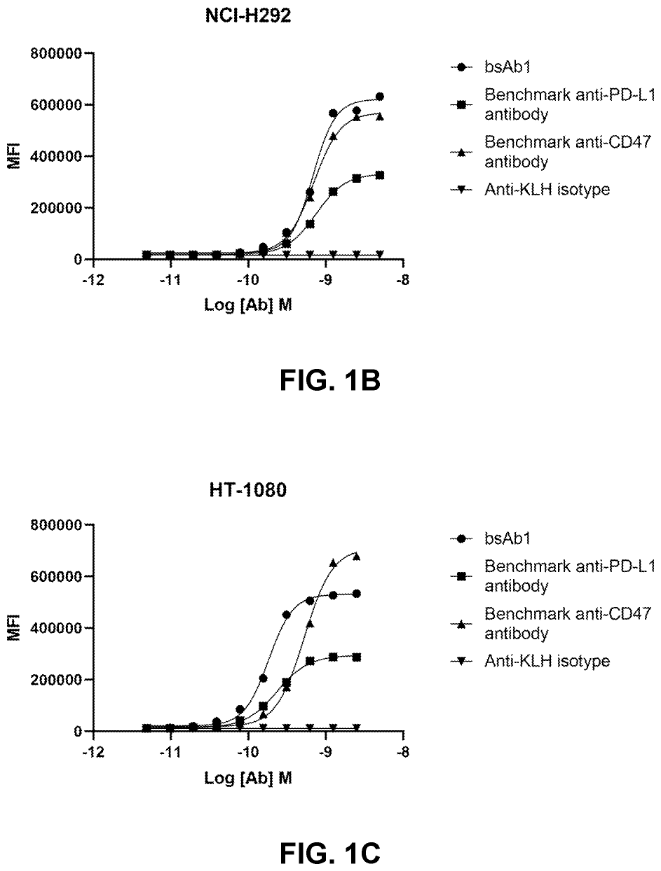

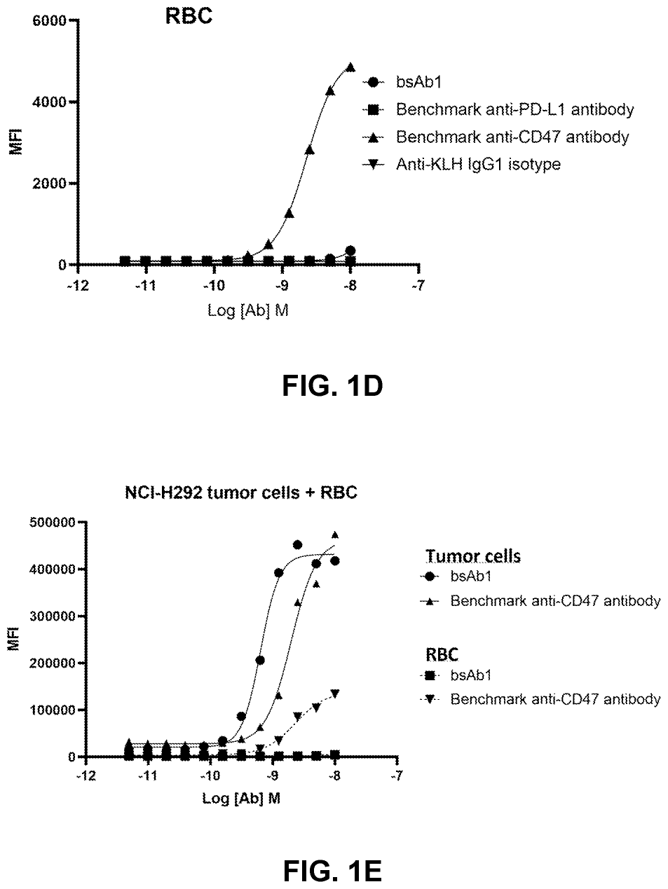

Bispecific antibody binding was tested in MDA-MB-231 cells expressing PD-L1 (PDL1-MDA-MB-231 cells), which have a CD47 surface protein copy number of approximately 500,000 and a PD-L1 surface protein copy number of approximately 1,500,000. For these experiments, a preparation of bispecific antibody was tested for cell binding. Cells were harvested and assayed for binding as described above. Exemplary binding curves are set forth in FIG. 1A. The EC₅₀ value of the selected bispecific antibody binding to PDL1-MDA-MB-231 cells is 0.22 nM. Two additional preparations were made using methods similar to those described in Example 1 and gave similar results.

Additional cell binding assays were performed evaluating bindings of the selected bispecific antibody in an exemplary format (designated bsAb1 herein) to tumor cell lines endogenously expressing both CD47 and PD-L1, for example, NCI-H292 cells and HT-1080 cells. Except for the tested cells, the experimental settings were the same as the cell binding assay with PDL1-MDA-MB-231 cells engineered to express PD-L1 as detailed above. A benchmark anti-PD-L1 antibody, a benchmark anti-CD47 antibody, and an anti-KLH isotype were also tested in parallel to bsAb1, serving as controls.

Co-expression of CD47 and PD-L1 on H292 cells and HT-1080 cells was confirmed, and an exemplary result is provided in Table 4 below.

TABLE 4
Co-expression of CD47 and PD-L1
on H292 Cells and HT-1080 Cells

| | PD-L1 | CD47 | CD47/PD-L1 |
|---|---|---|---|
| Cell line | copy # | copy # | ratio |
| NCI-H292 | 106,153 | 92,171 | 0.9 |
| HT-1080 | 29,843 | 21,971 | 0.7 |

Exemplary binding curves are set forth in FIG. 1B (NCI-H292) and FIG. 1C (HT-1080). The EC₅₀ values of bsAb1 were calculated and are provided in Table 5 below. These results indicate that bsAb1 showed strong binding on NCI-H292 and HT-1080 cells endogenously expressing both CD47 and PD-L1.

TABLE 5
EC₅₀ Values of bsAb1

EC₅₀ (nM)

| | NCI-H292 | HT-1080 |
|---|---|---|
| bsAb1 | 0.7 | 0.2 |
| Benchmark | 0.7 | 0.2 |
| anti-PD-L1 antibody | | |
| Benchmark | 0.7 | 0.5 |
| anti-CD47 antibody | | |
| Anti-KLH isotype | No activity | No activity |

Further, the selected bispecific antibody in an exemplary format (designated bsAb1 herein) was tested in binding to red blood cells (RBCs) alone, and tumor cells endogenously expressing both CD47 and PD-L1 (for example, NCI-H292 cells) in the presence of RBCs.

For the assay with RBCs, 100,000 fresh human erythrocyte cells (SER-10MLRBC-SDS, Zen-Bio) were transferred to each well of a 96-well V-bottom plate. The plate was then centrifuged at 500×g for 5 minutes at room temperature and the supernatant was discarded. The tested antibody was diluted in BD Stain Buffer (BD Biosciences) and 100 μL was transferred to each well of the plate. The plate was then incubated for 45 minutes at 4° C. Following the incubation, 100 μL PBS was added to each well. The plate was centrifuged at 500×g for 5 minutes at room temperature, and the supernatant was discarded. Next, 100 μL anti-human IgG Fab Alexa Fluor 488 (Jackson Immuno Research 109-547-003) diluted at 1:100 in BD Stain Buffer was added to each well. The plate was incubated for 45 minutes at 4° C., centrifuged at 500×g for 5 minutes at room temperature, and the supernatant was discarded. Cells were washed once with 200 L/well PBS and resuspended in 50 μL/well PBS. Samples were analyzed by flow cytometry on a Sartorius iQue Screener Plus instrument.

For the assay with tumor cells in the presence of RBCs, the tumor cell line (NCI-H292) was harvested at 70-90% confluence on the day of the assay. Cells (2.5×10⁴ per well in 25 μL, i.e., 1 million cells/mL) were collected with overage and by resuspension in 1×DPBS to reach a density of 1 million cells/mL. CellTrace Violet stock solution was prepared immediately prior to use according to the manufacturer's instruction. An appropriate volume of stock CellTrace Violet was added to the cell suspension to reach a final concentration of 1 μM. The cells were then incubated with dye at 37° C. for 20 minutes. 5× volume of BD stain buffer was added, and the sample was spun down at 300×g for 5 minutes to collect labeled cells. Cell pellet was resuspended in the same volume of 1×DPBS as described above. 25 μL was seeded in appropriate wells according to a plate map and the plate was left on ice. 450 μL RBC was removed from stock and diluted in 45 mL DPBS. 5 mL was removed from the diluted RBC/DPBS and further diluted in 45 mL before cell count. An appropriate number of RBC was collected from diluted RBC stock and resuspended in BD staining buffer at a density of 50 million cells/mL (1:50 mix) or 100 million cells/mL (1:100 mix). 25 μL of RBC was added to appropriate wells according to the plate map and the plate was placed on ice. Serial dilution of test antibodies was performed. 50 μL of test antibodies was added per well for the binding assay. The prepared plate was incubated on ice for 1 hour before spinning at 400×g for 5 minutes and the supernatant was discarded. The cells were then rinsed with 200 μl DPBS and the plate was spun down at 400×g for 5 minutes and the supernatant was discarded. Goat anti-human Alexa Fluor 488 200× was diluted and 50 μL was added to each well. After incubation on ice for 30 min, the plate was centrifuged at 400×g for 5 minutes and the supernatant was removed. The samples were washed once with 200 μL DPBS. Cells were resuspended in 100 μL PBS before analyzing on the iQUE3 flow cytometer.

Exemplary binding curves are set forth in FIG. 1D (RBC) and FIG. 1E (NCI-H292+RBC). The result shows that bsAb1 selectively bound to tumor cells in presence of RBCs, and this binding selectivity of bsAb1 was much higher compared to a benchmark anti-CD47 antibody.

### Example 3. Functional Assays: Inhibiting CD47/SIRPα & PD-1/PD-L1 Signaling

The selected bispecific antibody in an exemplary format (designated bsAb1 herein) was evaluated for inhibition of a CD47/SIRPα signaling and PD-1/PD-L1 signaling in cell types expressing CD47 and PD-L1, for example, MDA-MB-231 cells engineered to express PD-L1 (referred to herein as PDL1-MDA-MB-231). Co-expression of CD47 and PD-L1 on PDL1-MDA-MB-231 cells was confirmed, and an exemplary result is shown in Table 6 below.

TABLE 6
Expression of CD47 and PD-L1 on PDL1-MDA-MB-231 Cells

| | PD-L1 | CD47 | PD-L1/CD47 |
|---|---|---|---|
| Cell line | copy # | copy # | ratio |
| PDL1-MDA-MB-231 | 1,800,000 | 550,000 | 3.3 |

To test the ability of the bispecific antibody to inhibit CD47/SIRPα signaling, the bispecific antibody as described herein was assayed using a CD47/SIRPα Signaling Bioassay Kit (93-1135C19, Eurofins DiscoverX) following the manufacturer protocol. The assay was conducted using PDL1-MDA-MB-231 target cells.

The signaling assays are engineered to co-express a ProLink™ (PK) tagged immune checkpoint receptor and an Enzyme Acceptor (EA) tagged SH2 domain. Ligand engagement leads to receptor activation and phosphorylation, resulting in SH2-EA recruitment to the receptor, and forcing complementation of the two β-galactosidase enzyme fragments (EA and PK). The resulting functional enzyme hydrolyzes substrate to generate a chemiluminescent signal. Blocking of the ligand engagement leads to a drop in chemiluminescent signal.

Briefly, 30,000 target cells/wells in 40 μL Cell Plating Reagent were added into a White Bottom 96-well Plate (Corning 3917). Antibody dilutions were prepared in Cell Plating Reagent and 20 μL of antibody dilution were added to target cells. Next, 10,000 freshly thawed SIRPα Jurkat bioassay cells (PathHunter® Jurkat SIRPα Signaling Cell Line, Enrofins, Catalog No. 93-1135C19) in 40 μL Cell Plating Reagent were added to each well. The plate was incubated for 24 hours at 37° C. Following incubation, 10 μL BioAssay Reagent 1 was added to each well and the plate was incubated for 15 minutes at room temperature in the dark. Then, 40 μL BioAssay Reagent 2 was added to each well and the plate was incubated for 1 hour at room temperature in the dark. The plate was then read on a ClarioStar Plate Reader (BMG Labtech) or equivalent. More details of the assay can be found in the user manual titled PathHunter® Checkpoint Signaling Assay-Immunoglobulin Superfamily (IgSF) with a Document Number 70-394 Rev 3, dated 2020, and accessible at www.discoverx.com/tools-resources/document-resource-library/documents/user-manual-pathhunter % C2% AE-checkpoint-signaling-ass.

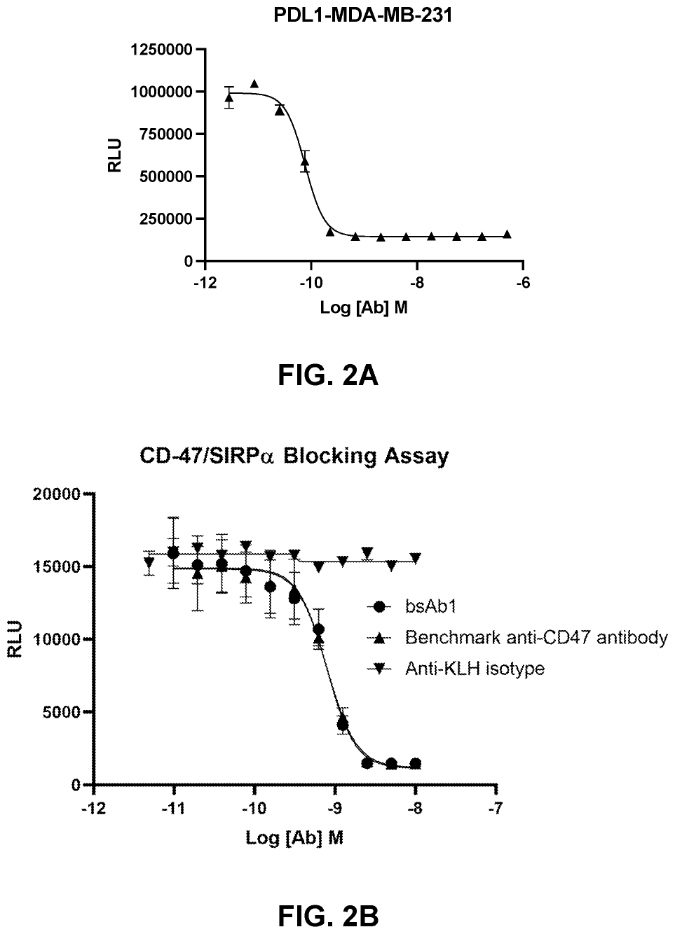

Exemplary results of the CD47/SIRPα signaling assays with PDL1-MDA-MB-231 cells are set forth in FIG. 2A. The EC₅₀ value of the selected bispecific antibody inhibiting CD47/SIRPα signaling in PDL1-MDA-MB-231 cells was 0.076 nM. Two additional preparations were made using methods similar to those described in Example 1 and gave similar results. In some embodiments, a benchmark anti-CD47 antibody and an anti-KLH isotype were further tested in parallel to the bsAb1, severing as controls. The corresponding curve is shown in FIG. 2B, while the calculated IC₅₀ values are provided in Table 7 below. These results indicate that the selected bispecific antibody showed a reduction in the chemiluminescent signal. Thus, the bispecific antibody efficiently blocked the engagement of CD47 ligand on the SIRPα receptor, inhibiting signaling (e.g., releasing the CD47/SIRPα checkpoint blockade).

TABLE 7
IC₅₀ Values

| CD47/SIRPa block | IC₅₀ (nM) |
|---|---|
| bsAb1 | 0.8 |
| Benchmark | 0.8 |
| anti-CD47 antibody | |
| Anti-KLH IgG1 | Not active |
| isotype | |

To test the ability of the bispecific antibody to inhibit PD-1/PD-L1 signaling, the bispecific antibody as described herein was assayed using a PD-1/PD-L1 Signaling Bioassay Kit (93-1104Y19-00117, Eurofins DiscoverX) following the manufacturer protocol. The assay was conducted using PDL1-MDA-MB-231 target cells.

Briefly, 40 μL of target cells (30,000 cells per 40 μL) was added to each well of a white 96-well plate in Cell Plating 0 Reagent (CPO). The plate was incubated at 37° C. overnight. PD-1 Jurkat (PathHunter® Jurkat PD-1 (SHP1) Signaling Cell Line, Enrofins, Catalog No. 93-1104C19) cells were thawed for overnight recovery from thaw following the procedures detailed below. 9.6 mL of pre-warmed Cell Plating Reagent (CPO) were added to a T25 flask. Two cryovials of PD-1 Jurkat cells were removed from liquid nitrogen. The frozen pellet was thawed by immediately adding 1 mL of pre-warmed CPO from the T25 flask to the cryovial. Cells were mixed by gently pipetting up and down several times to break up any clumps. The cell suspension was transferred to the T25 flask containing the remaining CPO and any media/suspension left in the cryovial was removed to ensure complete recovery of all the cells from the vial. The thawed PD-1 Jurkat cells were incubated at 37° C. overnight.

Following the incubation, 20 μL of serially diluted tested antibody was added to each well of the target cell plate and the plate was incubated at 37° C. for 1 hour. Then, 40 μL of PathHunter PD-1 Jurkat cells in CPO was added to each well of the 96-well plate and the plate was incubated at room temperature in the dark for 1 hour. Then, 10 UL of Bioassay Detection Reagent 1 was added to each well and the plate was incubated for 15 minutes at room temperature in the dark. Next, 40 μL of Bioassay Detection Reagent 2 was added to each well of the assay plate and the plate was incubated for 3 hours at room temperature in the dark. The plate was then analyzed on a ClarioStar Plate Reader (BMG Labtech) or equivalent. More details of the assay can be found in the user manual titled PathHunter® Checkpoint Signaling Assay-Immunoglobulin Superfamily (IgSF) with a Document Number 70-394 Rev 3, dated 2020, and accessible at www.discoverx.com/tools-resources/document-resource-library/documents/user-manual-pathhunter % C2% AE-checkpoint-signaling-ass.

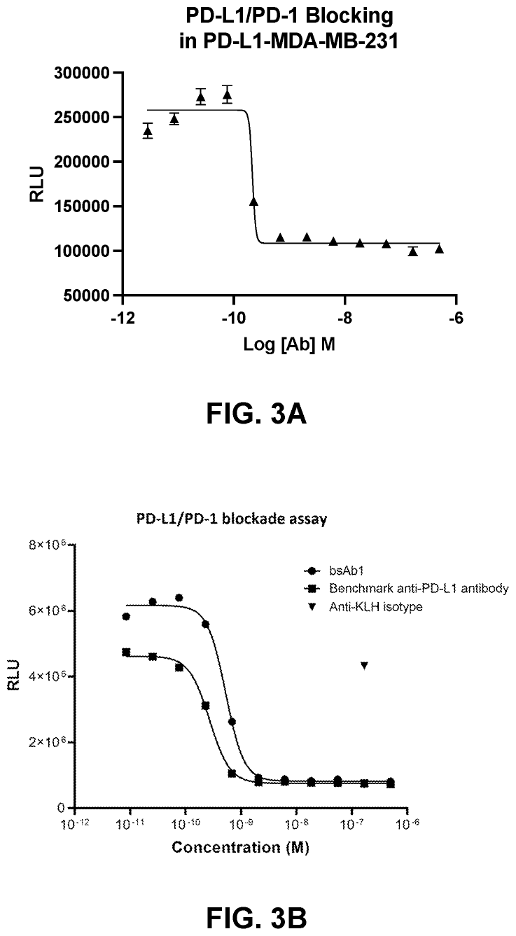

Exemplary results of the PD-1/PD-L1 checkpoint signaling assay in PDL1-MDA-MB-231 cells are shown in FIG. 3A. The EC₅₀ value of the selected bispecific antibody inhibiting PD-1/PD-L1 interaction in PDL1-MDA-MB-231 cells was 0.22 nM. Two additional preparations were made using methods similar to those described in Example 1 and gave similar results. In some embodiments, a benchmark anti-PD-L1 antibody and an anti-KLH isotype were further tested in parallel to the bsAb1, severing as controls. The corresponding curve is shown in FIG. 3B, while the calculated IC₅₀ values are provided in Table 8 below. Results indicate that the selected bispecific antibody showed a reduction in the chemiluminescent signal. Thus, the bispecific antibody efficiently blocked the interaction of PD-L1 and PD-1, inhibiting signaling (e.g., releasing the PD-L1/PD-1 checkpoint blockade).

TABLE 8
IC₅₀ Values

| PD-L1/PD-1 block | IC₅₀ (nM) |
|---|---|
| bsAb1 | 0.5 |
| Benchmark anti-PD-L1 | 0.3 |
| antibody | |
| Anti-KLH IgG1 isotype | Not active |

### Example 4. Additional Functional Assays: Phagocytosis (ADCP) and ADCC

The selected bispecific antibody in an exemplary format (designated bsAb1 herein) was tested for its ability to promote phagocytosis. A macrophage phagocytosis assay was performed with JeKo-PD-L1 cells as target cells that were exposed to macrophages in the presence of the selected bispecific antibody.

Macrophages were derived from human monocytes by monocyte differentiation in stimulatory culture medium. Briefly, freshly thawed CD14+ monocytes were transferred into a 50 mL tube and diluted with 25 mL RPMI culture medium (RPMI (61870-036, Gibco), 10% FBS (10437-028, Gibco), 1% Pen/Strep (15140-122, Gibco), 1% NEAA (non-essential amino acids) (11140-050, Gibco), 1% Sodium Pyruvate (11360-070, Gibco)). Cell suspensions were centrifuged at 400×g for 10 minutes at 4° C. Following monocyte cell resuspension and counting, the CD14+ monocytes were seeded at 1×10⁶ cells per mL in a 75 mm³ flask containing 12 mL of ImmunoCult™-SF Macrophage Medium (10961, Stemcell Technologies). On day 4, 50% of the culture media was replaced with fresh culture media with 50 ng/ml macrophage colony-stimulating factor (M-CSF) (78057, Stemcell Technologies) and 10 ng/mL IL-4 (78045.1, Stemcell Technologies). On day 6, the monocyte-derived macrophages were harvested with TrypLE Express (12605010, Thermo Fisher) then counted and plated at 5×10⁴ cells per well in 50 μL per well of RPMI medium with a benchmark anti-CD20 antibody added to 20 nM into a half area clear bottom black plate (Corning 3882). The assay plate was incubated for at least 1 hour to let the macrophages attach. At this step, Jeko-PD-L1 cells expressing PD-L1 and CD47 used as target cells were labeled with pHrodo Red Cell Labeling Kit (A10026, ThermoFisher Scientific) according to the manufacturer protocol. Briefly, 20 million cells were harvested and washed once in 20 mL buffer A, then centrifuged and resuspended in 20 mL buffer B from the labeling kit. Following cell resuspension, 2 μL pHrodo Red dye at 1 mg/mL was added and incubated at 37° C. for 1 hour. Cells were centrifuged, washed once with RPMI, and then resuspended in RPMI.

To 2× antibody dilutions (1, 0.25, 0.0625, 0.0156, 0.0039, 9.7E-4, 2.44E-4, 6.1E-5, 1.5E-5, 3.8E-6 and 9.53E-7 μM) prepared in RPMI, labeled Jeko-PD-L1 at 2×10⁶ cells per mL were added and incubated at 37° C. for 30 minutes (to allow antibodies binding to cell surface CD47). Following incubation, 50 μL per well of the 2× antibody dilution containing 100,000 labeled Jeko-PD-L1 cells were transferred to the assay plate containing pre-seeded macrophages. The assay plate was incubated in the Incucyte live-cell imaging system (Sartorius). Phase and fluorescent images were taken every 30 minutes or 1 hour intervals for a total of 24 hours, at 20× high magnification, 800 ms scan in red channel. Signals from wells with only pHrodo Red dye labeled target cells were used for Incucyte signal appropriation, in which the red scale was manually adjusted for minimum and maximum signal in such a way that the wells with pHrodo Red labeled target cells alone showed no signal. Wells with pHrodo Red dye labeled target cells co-culturing with macrophages without any anti-CD47 antibody were used as assay negative control and basis of minimum mean fluorescence intensity cut-off.

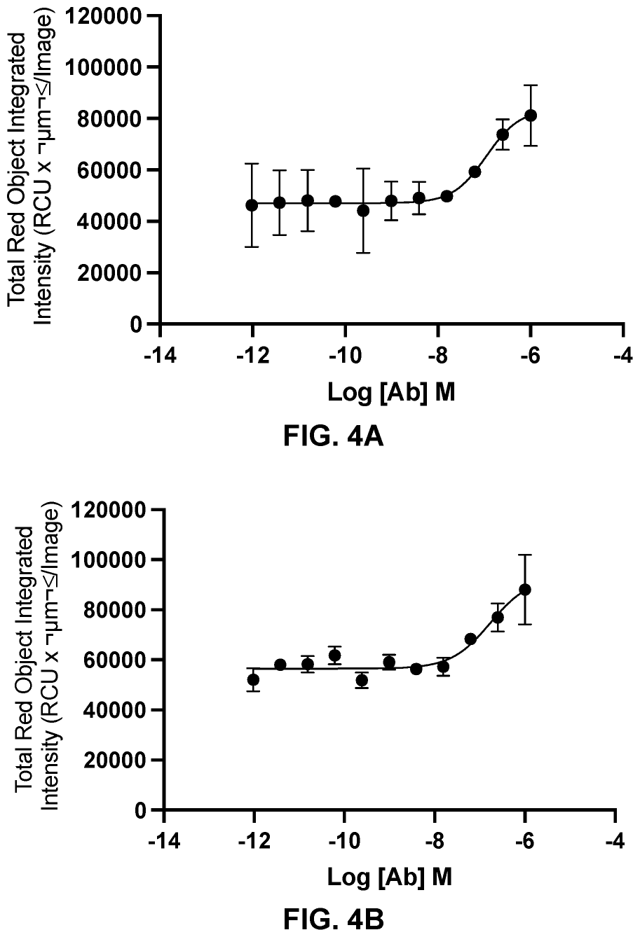

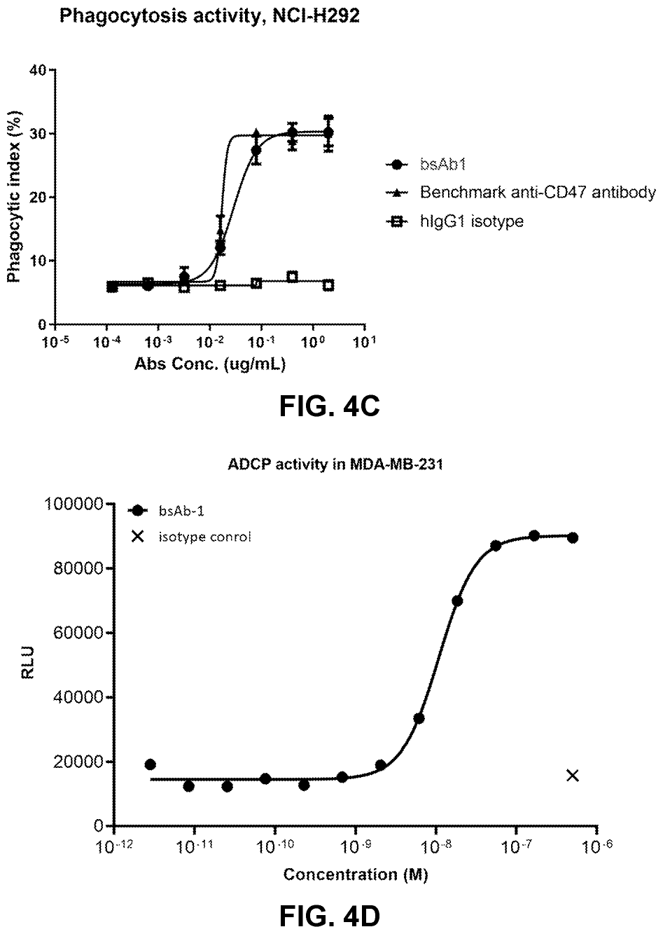

Exemplary results of fluorescent object intensity are shown in FIGS. 4A (bsAb1) and 4B (bsAb1 with silent Fc). Results indicate that the intensity of red signal increases where the pHrodo Red dye labeled target cells are exposed to the macrophages in the presence of the selected bispecific antibody. Results suggest that the selected bispecific antibody promotes the macrophage phagocytosis of the target cells.

Additionally, the selected bispecific antibody in an exemplary format (designated bsAb1 herein) was tested for its ability to promote phagocytosis with tumor cell lines endogenously expressing both CD47 and PD-L1 as target cells, for example, NCI-H292 cells. The experiments were detailed below, and an anti-CD47 antibody and a human IgG1 (hIgG1) isotype were also tested in parallel to bsAb1, serving as controls.

Induction of monocyte to macrophage: human peripheral blood mononuclear cells (PBMCs) were cultured in a 10-cm dish for 2 hours (1-2×107/dish) with 10 mL RPMI1640 culture medium. The culture medium was then replaced with fresh cytokine-containing medium (RPMI1640+10% FBS+20 ng/ml rHuM-CSF). On day 3 and day 5, the medium was again replaced with fresh cytokine-containing medium (RPMI1640+10% FBS+20 ng/ml rHuM-CSF). On day 7, adherent macrophages were detached from the dish by incubation at 37° C. with TrypLE. Cells were washed twice with PBS or sterile FACS buffer (PBS, pH7.4+2% FBS) and seeded on a flat-bottom 96-well plate (Corning Cat #3599) at 4×10⁴/well in 100 μL fresh cytokine-containing medium (RPMI1640+10% FBS+20 ng/ml rHuM-CSF+10 ng/ml rHuIL-10) overnight.

CFSE labeling of tumor cells: NCI-H292 cells were harvested and washed twice with PBS, and then labeled at 1×10⁶ cells/mL in PBS with 2 M CFSE at room temperature for 8 minutes. CFSE labeling was terminated by adding an equal volume of 100% FBS and incubating at 37° C. for 10 minutes. Cells were then washed twice with an FACS buffer and resuspended in an assay buffer (RPMI1640+10% FBS) to a final concentration of 4×10⁵/mL.

Phagocytosis: The medium of adherent macrophages on the flat-bottom 96-well plate was removed and the cells were washed twice with PBS. Various concentrations of the tested antibodies and isotype control (serially diluted in assay buffer) were independently added at 50 μL/well. 50 μL CFSE labeled tumor cells were then added at a T:E ratio of 1:2 and the plate was incubated for 4 hours. All cells were then detached with TrypLE and washed twice with PBS. Next, the cells were stained with anti-CD14 at 4° C. for 60 minutes in the dark. After the incubation, cells were washed twice with PBS. Cells were then suspended in a FACS buffer and analyzed for CD14+CFSE+ population by flow cytometry. The phagocytosis index was calculated as the percentage of CFSE+CD14+ cells over all CD14+ cells, following the equation below.

Phagocytosis Index=100×Q2 Events/(Q2+Q4) Total Events Q2: CD14+CFSE+, Q4: CD14+CFSE−, Q1: CD14−CFSE+, and Q3: CD14−CFSE−.

Co-expression of CD47 and PD-L1 on H292 cells was confirmed, and an exemplary result is shown in Table 4 provided herein.

An exemplary binding curve is set forth in FIG. 4C. The EC₅₀ values of bsAb1 were calculated and are provided in Table 9 below. These results indicate that bsAb1 exhibited potent phagocytosis activity (ADCP) on NCI-H292 expressing PD-L1 and CD47.

TABLE 9
Exemplary Binding Curve

| | EC₅₀ | |
|---|---|---|
| ADCP | (mg/mL) | EC₅₀ (nM) |
| bsAb1 | 0.03 | 0.2 |
| Benchmark | 0.02 | 0.1 |
| anti-CD47 antibody | | |
| hIgG1 | Not active | Not active |

The selected bispecific antibody in an exemplary format (designated bsAb1 herein) was further tested using a second antibody-dependent cellular phagocytosis (ADCP) bioassay.

ADCP reporter-based bioassay was performed using FcγRIIa-H Effector Cells (Promega, G998A). Target cells, (e.g., MDA-MB-231), were resuspended in pre-warmed (37° C.) assay medium (RPMI1640+4% low IgG, Promega, G711A and G708A). 25 μL per well of target cells with indicated effector:target (E:T) ratios were dispensed on a 96-well microplate (Thermo Fisher Scientific, 136101), followed by an overnight incubation (for adherent cells) or a 15-minute equilibration (for suspension cells) in a 37° C., 5% CO₂ incubator. Serial dilution of bsAb1 at indicated concentrations was prepared in pre-warmed (37° C.) assay medium. 25 μL per well of bsAb1 were added into the 96-well microplate and mixed gently with the plated target cells. Effector cells were removed from storage at −140° C. and immediately thawed in a 37° C. water bath with gentle agitation, followed by resuspending in pre-warmed (37° C.) assay medium at 80,000 cells/mL. 25 L per well of effector cells (e.g., 20,000 cells) were added into the 96-well microplate and mixed gently. Any empty wells were added with 75 μL assay medium. The assay plate was covered with a lid and incubated in 37° C., 5% CO₂ incubator overnight. After incubation, the assay plate was removed from the incubator and equilibrated to ambient temperate for 15 minutes. Bio-Glo™ reagent (Promega, G719A, G720A) was prepared according to the manufacturer's instructions. 75 μL per well of Bio-Glo™ reagent were added into the 96-well microplate and incubated for 5 minutes at ambient temperature. Luminescence measurements (RLU) were obtained using a luminescence plate reader (Perkin EnVision). Curves fitting and EC₅₀ of bsAb1 responses were analyzed using GraphPad Prism.

An exemplary result is plotted in FIG. 4D. EC₅₀ was calculated as 1.1×10⁻⁸ M (i.e., 11 nM).

Additionally, the selected bispecific antibody in an exemplary format (designated bsAb1 herein) was tested for its ability to induce antibody-dependent cellular cytotoxicity (ADCC).

ADCC reporter-based bioassay was performed using ADCC Bioassay Effector Cells (Promega, G701A). Target cells (such as, MDA-MB-231), were resuspended in pre-warmed (37° C.) assay medium (RPMI1640+4% low IgG, Promega, G711A and G708A). 25 μL per well of target cells with indicated effector:target (E:T) ratios were dispensed on a 96-well microplate (Thermo Fisher Scientific, 136101), followed by an overnight incubation (for adherent cells) or a 15-minute equilibration (for suspension cells) in a 37° C., 5% CO₂ incubator. Serial dilution of bsAb1 at indicated concentrations was prepared in pre-warmed (37° C.) assay medium. 25 μL per well of bsAb1 were added into the 96-well microplate and mixed gently with the plated target cells. Effector cells were removed from storage at −140° C. and immediately thawed in a 37° C. water bath with gentle agitation, followed by resuspending in pre-warmed (37° C.) assay medium at 80,000 cells/mL. 25 μL per well of effector cells (e.g., 20,000 cells) were added into the 96-well microplate and mixed gently. Any empty wells were added with 75 μL assay medium. The assay plate was then covered with a lid and incubated in a 37° C., 5% CO₂ incubator overnight. After incubation, the assay plate was removed from the incubator and equilibrated to ambient temperate for 15 minutes. Bio-Glo™ reagent (Promega, G719A, G720A) was prepared according to the manufacturer's instructions. 75 μL per well of Bio-Glo™ reagent were added into the 96-well microplate and incubated for 5 minutes at ambient temperature. Luminescence measurements (RLU) were obtained using a luminescence plate reader (Perkin EnVision). Curves fitting and EC₅₀ of bsAb1 responses were analyzed using GraphPad Prism.

### Example 5. Red Blood Cell Hemagglutination Assay

Anti-CD47 antibodies may cause red blood cell agglutination, which limits their therapeutic applications. The selected bispecific antibody in an exemplary format (designated bsAb1 herein) was tested in an assay to determine its effect on red blood cell agglutination.

For these assays, fresh human whole blood from each of three donors was diluted with equal volume of PBS without Ca²⁺ and Mg²⁺ (PBS) and centrifuged at 2000 rpm/min for 5 minutes. The pelleted red blood cells were washed three times with PBS and diluted to 5% in PBS for assay use. The selected bispecific antibody as described herein was prepared in a 3-fold serial titration at concentrations ranging from 0.002 to 100 mg/mL. Diluted red blood cells from each of the three donors were incubated with titrated antibodies in a round-bottomed 96-well plate for 2 to 6 hours at 37° C. Hemagglutination was demonstrated by the presence of non-settled red blood cells appearing as a haze compared to punctuate red dot of non-hemagglutinated red blood cells. Hemagglutination index was determined by quantitating the area of red blood cell pellet in the presence of the selected bispecific antibody, in which the area was normalized by the area of the red blood cell pellet from wells without the selected bispecific antibody.

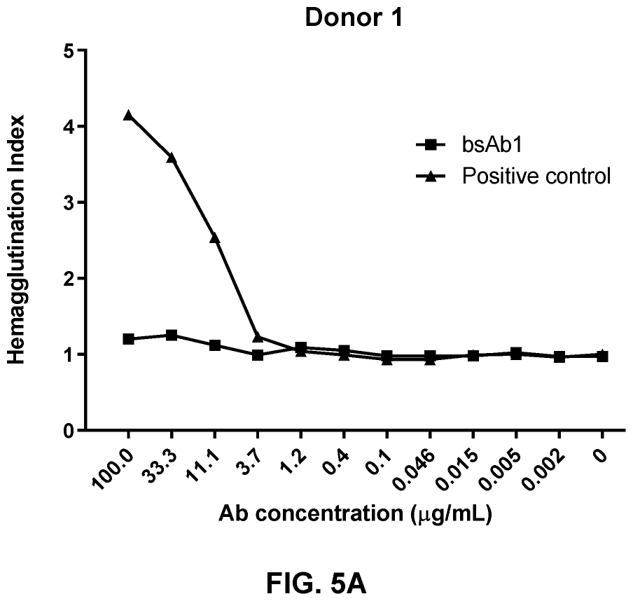

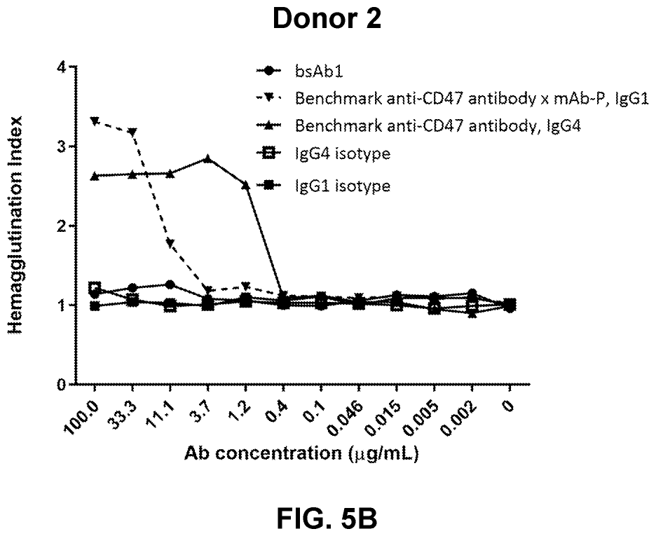

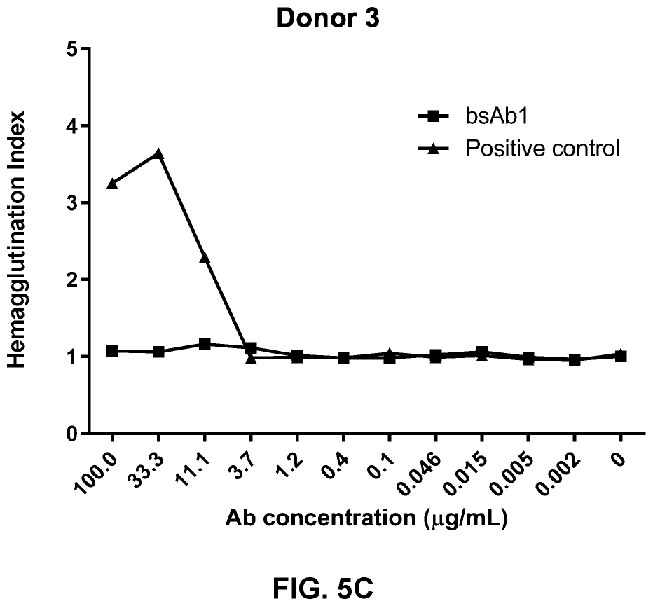

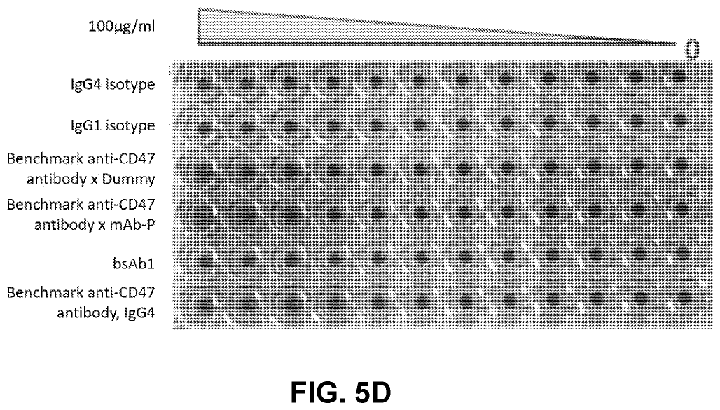

Exemplary results of hemagglutination assays are shown in FIGS. 5A-5C, each of which represents data obtained using human whole blood from one donor. Results indicate that the selected bispecific antibody resulted in no red blood cell agglutination compared to a positive control, which is predictive of little interaction with red blood cells in vivo. In this experiment, a bispecific antibody similar to bsAb1 (e.g., having a variable region of mAb-P and an IgG1 Fc) but having a variable region of a benchmark anti-CD47 antibody (instead of mAb-C) served as a positive control.

A further experiment was performed, including the following controls: (1) a bispecific antibody replacing the CD47 binding portion of the bsAb1 with the corresponding portion of a benchmark anti-CD47 antibody (referred to herein as a benchmark anti-CD47 antibody×mAb-P), (2) a benchmark anti-CD47 antibody having an IgG4 Fc, (3) an IgG4 isotype, (4) an IgG1 isotype, and (5) a bispecific antibody replacing the CD47 binding portion of the bsAb1 with the corresponding portion of a benchmark anti-CD47 antibody and the PD-L1 binding portion of the bsAb1 with the corresponding portion of a non-specific antigen such as KLH isotype (referred to herein as Benchmark anti-CD47 antibody×Dummy). A representative result using samples from one out of 3 donors is provided in FIG. 5D, indicating a lack of hemagglutination activity.

### Example 6. Developability Assays

The selected bispecific antibody in an exemplary format (designated bsAb1 herein) was tested in various developability methods. For example, various chromatographic methods, including size exclusion chromatography (SEC), hydrophobic interaction chromatography (HIC), and standup monolayer adsorption chromatography (SMAC) were employed to assess developability factors, such as monomer percentage, solubility, and antibody aggregation or precipitation.

Size exclusion chromatography (SEC) analysis was performed using a 7.8 mm ID×30 cm TSKgel G3000SWXL column (Tosoh Bioscience LLC, PN 08541) on an Agilent 1100 HPLC. The selected bispecific antibody as described herein was normalized to 1 mg/mL concentration in Dulbecco's PBS (pH 7.4, without Ca2+/Mg2+) and clarified via centrifugation to pellet particulates while still retaining soluble aggregates. The mobile phase buffer was Dulbecco's PBS (pH 7.4, without Ca2+/Mg2+). 10 μL sample was loaded and isocratically eluted at 1.0 mL/min over 20 minutes. Absorbance was monitored at 280 nm. Chromatographic peaks were integrated to determine % homogeneity and retention time. The column stationary phase along with choice of mobile phase supports hydrophobic interaction in addition to molecular sizing (hydrophobic interaction much milder compared to SMAC). Data analysis was performed using Agilent ChemStation B.04.03.

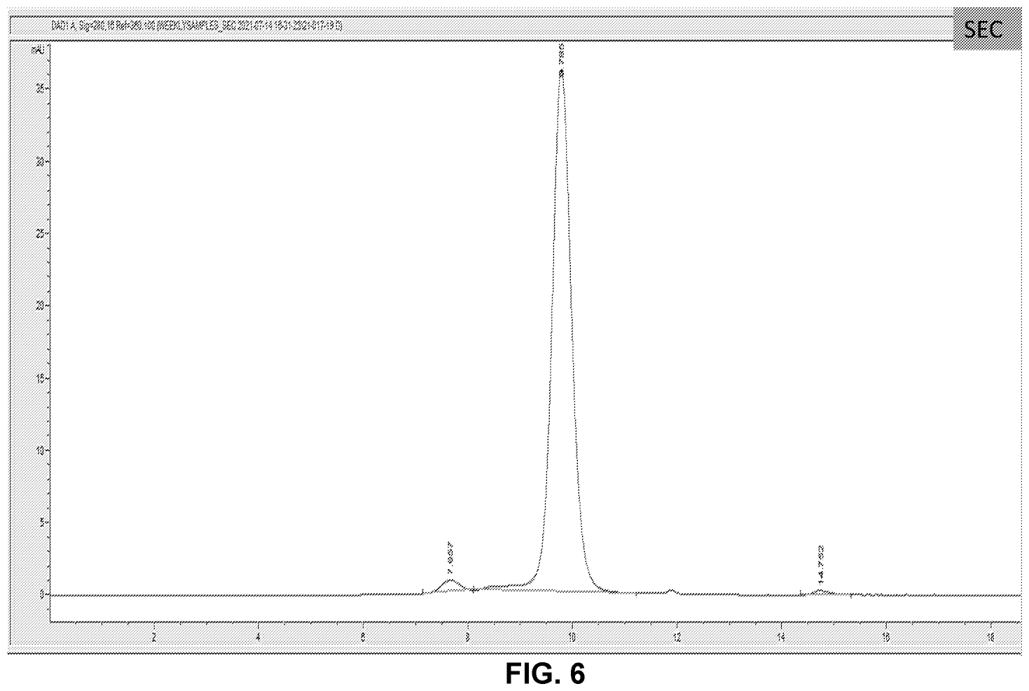

Exemplary SEC results are shown in FIG. 6. Results indicate low bispecific antibody aggregation.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. The selected bispecific antibody as described herein was normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. 10 μL sample was loaded and eluted using a gradient from 100% mobile phase buffer B to 100% mobile phase buffer A (20 mM sodium phosphate, pH 7.0) at 1.0 mL/min over 15 min, held at 100% A for 3 minutes to wash the column, and returned 100% B for 2 minutes for equilibration. Absorbance was monitored at 280 nm. Sample retention time was calculated and compared to a set of standard controls to identify bispecific antibodies with increased retention time (increased hydrophobicity).

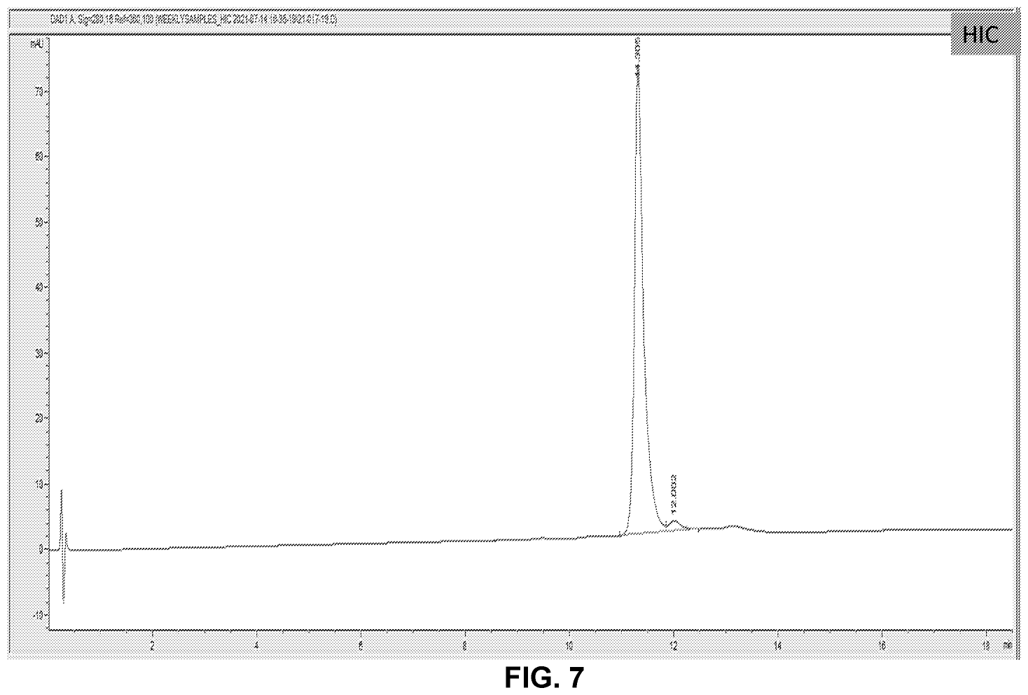

Exemplary HIC results are shown in FIG. 7. Antibody hydrophobicity can impact antibody aggregation, solubility and viscosity. Results show similar retention times for the selected bispecific antibody, and indicate a low propensity for aggregation and precipitation. The HIC elution profiles (e.g., sharpness of elution peak and uniform retention times) of the selected bispecific antibody suggested low propensities to aggregate and/or precipitate.

Standup monolayer adsorption chromatography (SMAC) analysis was performed using a 4.6 mm ID×300 mm Zenix SEC 300 column (Sepax Technologies, PN 213300P-4630) on an Agilent 1100 HPLC. The selected bispecific antibody as described herein was normalized to 1 mg/ml concentration in dPBS (pH 7.4) and clarified via centrifugation to pellet particulates. The mobile phase buffer was dPBS (pH 7.4, without calcium and magnesium). 10 μL sample was loaded and isocratically eluted at 0.25 mL/min over 32 min. Absorbance was monitored at 280 nm. Sample retention time was calculated and compared to a set of standard controls to identify bispecific antibodies with increased retention time (increased propensity to form aggregates).

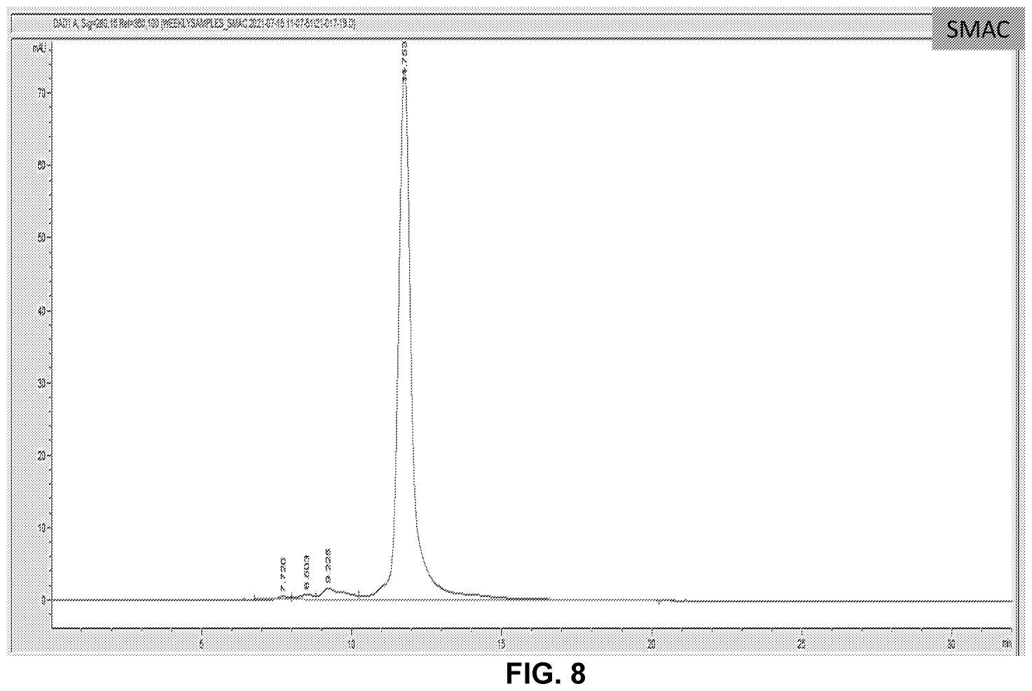

Exemplary SMAC results are shown in FIG. 8. Results show that the selected bispecific antibody had similar short retention times, indicating colloidal stability and low propensity to aggregate.

Overall, the results of the developability assays show that the developability criteria are met for the selected bispecific antibody.

### Example 7. In Vivo Efficacy Study

The selected bispecific antibody in an exemplary format (designated bsAb1 herein) was evaluated for its in vivo efficacy in the treatment of subcutaneous B-hPD-L1 plus/hCD47 MC38 colon carcinoma model in B-hPD-L1/hSIRPA/hCD47 mice.

Animals: Female B-hPD-L1/hSIRPA/hCD47 C57BL/6 mice (body weight of 15-21 g and 6-10 weeks old) were obtained and housed in a specific pathogen free (SPF) barrier in individual ventilated cages (IVC). Temperature and humidity were controlled as listed below: Temperature: 20-26° C.; Humidity: 40-70%; and Lighting: every 12 hours. The bedding material was pressure sterilized corncob, which was changed once per week. Free access to autoclaved sterilized dry granule food and water was provided to the animals during the entire study period. Food was SPF grade and purchased from Beijing KeaoXieli Feed Co., Ltd. Water was purified by ultrafiltration. Ear tag was used for animal identification, and the animals were acclimatized for 7 days prior to the starting of the experiment.

Cell culture: An MC38 murine colon carcinoma cell line was purchased from Shunran Shanghai Biological Technology Co., Ltd. Cells were maintained in vitro as monolayer culture in Dulbecco's Modified Eagle's Medium (DMEM, Gibco™, Catalog No. 11965-092) supplemented with 10% heat inactivated Fetal Bovine Serum (FBS, AusGeneX, Catalog No. 20012027) at 37° C. in a humidified atmosphere of 5% CO₂. The B-hPD-L1 plus/hCD47 MC38 cells were genetically modified by knocking out the mouse Pdl1 and Cd47 genes, as well as by knocking-in the human PD-L1 and CD47 genes in MC38 cells. The CD47:PD-L1 expression ratio of the MC38-hPD-L1/hCD47 tumor cell line was measured as 5.

Tumor inoculation and animal grouping: Eighty (80) B-hPD-L1/hSIRPA/hCD47 mice were subcutaneously injected with B-hPD-L1 plus/hCD47 MC38 tumor cells (5×10⁵) in 0.1 mL Phosphate Buffered Saline (PBS, Gibco™, Catalog No. 20012027) in the right front flank for tumor development. Fifty-six (56) tumor-bearing animals were randomly enrolled into the seven study groups when the mean tumor size reached approximately 80-100 mm³. Each group consisted of 8 mice. Treatments were administrated on the same day of grouping (denoted as Day 0). Dulbecoo's Phosphate Buffered Saline, Calcium and Magnesium free (D-PBS, Corning, Catalog No. 21-031-CM) served as a vehicle control. The experiment was terminated 28 days after the first dosing, and the animals were euthanized with CO₂.

TABLE 10
Dosing regimen

| Group | Test | No. of | Dosages | Dosing | Dosing | Dosing |
|---|---|---|---|---|---|---|
| No. | article | animals | (mg/kg)* | Route | Frequency | frequency |
| 1 | Anti-HEL | 8 | 10 | intraperitoneal | once every | 6 |
| | IgG1 | | | injection (i.p.) | two days | |
| | isotype | | | | (Q2D) | |
| | control | | | | | |
| 2 | Benchmark | 8 | 10 | i.p. | Q2D | 6 |
| | anti-PD-L1 | | | | | |
| | antibody | | | | | |
| 3 | Benchmark | 8 | 10 | i.p. | Q2D | 6 |
| | anti-CD47 | | | | | |
| | antibody | | | | | |
| 4 | bsAb1 | 8 | 3 | i.p. | Q2D | 6 |
| 5 | bsAb1 | 8 | 10 | i.p. | Q2D | 6 |

*Dosing volume was administered based on body weight (10 μL/g).

Dosing solution preparation: The stock solution of the test articles was aliquoted and stored at −80° C. The material was thawed on the day of grouping animals, just prior to dosing. The vial contents were transferred to a clear conical bottom centrifuge tube and centrifuged at 5000×g for 5 minutes at room temperature. After centrifugation, the vial contents were transferred to a new clear conical tube according to the amount of test article required. The conical tube(s) were stored at 2-8° C. or on ice until time of use. Dilutions were prepared, if needed. All dilutions were performed using sterile, low endotoxin, Ca²⁺ and Mg²⁺ free Dulbecco's PBS. The dosing articles were then equilibrated to room temperature immediately prior to dosing.

Tumor measurement index as described below were evaluated. Tumor volume (TV): Tumor size was measured two times weekly in two dimensions using a caliper, and the volume was calculated in mm³ using the formula: V=0.5 a×b² where a and b were the long and short dimensions of the tumor, respectively. Body weight (BW): Animals were weighed before tumor inoculation and animal grouping, then two times per week during the experiment, and right before the endpoint of the experiment.

Routine observations: During the entire experiment, animals were monitored every day for their behavior and status, including but not limited to appearance of tumor ulceration, animal mental status, visual estimation of food and water consumption, and so on.

Drug evaluation index as described below were also evaluated. Tumor growth inhibition (TGI_{TV}) by tumor volume (TV): The tumor growth inhibition TGI_{TV} was calculated by the following equation:

$$T G {I}_{TV} \% = [ 1 - ( {T}_{i} - {T}_{0} ) / ( {V}_{i} - {V}_{0} ) ] \times 100 \% .$$

(Tᵢ: tumor volume of the treated groups at day i following treatment; T₀: tumor volume of the treated groups at day 0; Vᵢ: tumor volume of the vehicle group at day i following treatment; V₀: tumor volume of the vehicle group at day 0).

Statistical analysis: Results were represented by means and the standard error (Mean±SEM). Statistical analysis of TGI_{TV} was performed using One-way ANOVA with Dunnett's multiple comparisons test. P values <0.05 were regarded as statistically significant.

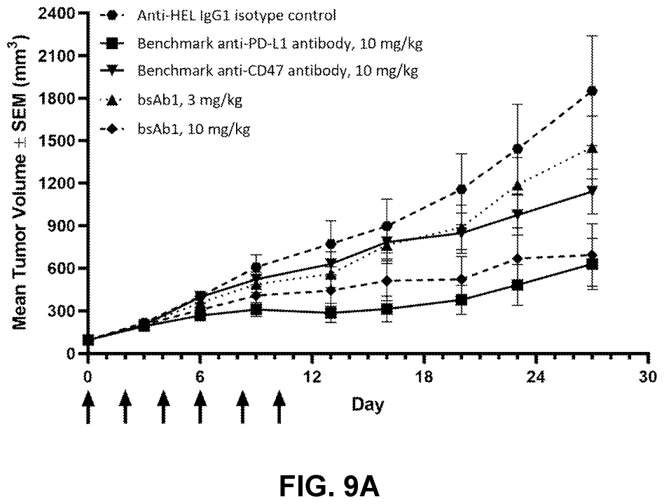

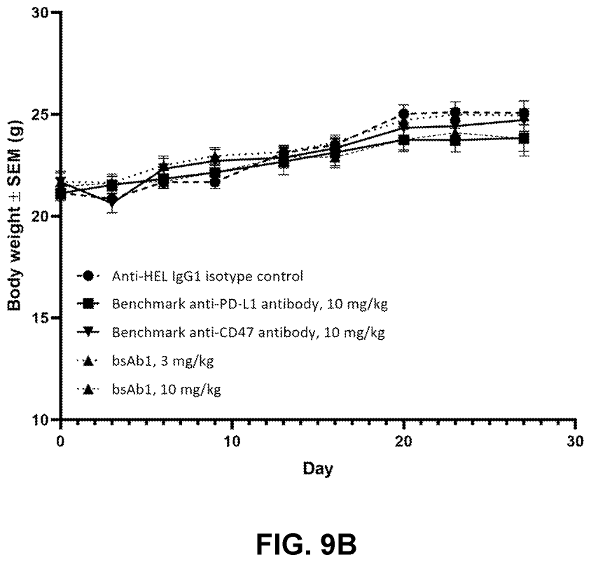

The results are plotted in FIGS. 9A-9B and provided in Table 11 below. No obvious clinical signs nor animal death were observed during the entire experiment and the body weights of all groups increased gradually during the duration of the study. These results indicate that bsAb1 is well-tolerated and demonstrated efficacy against MC38-hPD-L1/hCD47 tumor cells in hPD-L1/hCD47/hSIRP-α triple knock-in (KI) mouse model. bsAb1 at 10 mg/kg showed comparable effacay compared to benchmark anti-PD-L1 antibody (also dosed at 10 mg/kg).

TABLE 11
In Vivo Efficacy Study Results

| | | | P values |
|---|---|---|---|
| | | % Tumor Growth | as calculated |
| | Dose | Inhibition (TGI) | against isotype |
| Test Article | (mg/kg) | on Day 27 | control |
| Benchmark | 10 | 70 | 0.0060 |
| anti-PD-L1 | | | |
| antibody | | | |
| Benchmark | 10 | 41 | 0.2012 |
| anti-CD47 | | | |
| antibody | | | |
| bsAb1 | 3 | 23 | 0.7345 |
| | 10 | 66 | 0.0099 |

### Example 8. Non-Human Primate (NHP) Tolerability Data

The selected bispecific antibody in an exemplary format (designated bsAb1 herein) was evaluated to determine its tolerability profile when administered as a single dose via intravenous infusion to male cynomolgus monkeys. Accordingly, a 4-week single-dose intravenous infusion tolerability study with bsAb1 was performed in male cynomolgus monkeys.

Animals: Cynomolgus monkey was considered to be the pharmacologically relevant species because the drug has similar cross-reactivity to human and monkey target antigens. Weights of the animals at final pre-dose sampling were 2 to 3.5 kg; while ages of the animals at the initiation of dosing were 24 to 50 months. Animals were housed in stainless steel cages. When possible, animals were socially housed (up to three animals/cage). Animals were separated daily for the monitoring of individual clinical observations and qualitative food consumption, as outlined herein. Ad libitum water was accessible to the animals, while certified primate diet #5048 (PMI Nutrition International Certified LabDiet®) were provided one or twice daily. Environmental controls for the animal room were set to maintain a temperature range of 20 to 26° C., a relative humidity of 30 to 70%, a minimum of eight air changes/hour, and a 12-hour light/12-hour dark cycle. The light/light/dark cycle might be interrupted for study-related activities. Animals were given various cage-enrichment devices and dietary enrichment. A total of 6 males were acclimated for at least 2 weeks and then tested as described herein.

Dosing Preparation: bsAb1 was thawed in a refrigerator, set to maintain 2 to 8° C., up to 24 hours prior to use or, alternatively, at room temperature for up to 2 hours prior to use. The thawed bsAb1 might be stored in a refrigerator, set to maintain 2 to 8° C., for up to 4 days or stored (refrozen) in a freezer, set to maintain-60 to −80° C. In total, bsAb1 might undergo up to 3 freeze/thaw cycles. Dulbecco's Phosphate-Buffered Saline (pH 7.1 to 7.3 (1×), without calcium chloride, without magnesium chloride) was used as a diluent/vehicle control.

Dosing: A one-time dosing was performed, and Day 1 of the dosing phase was defined as the first day of dosing for each group. The dosing was via intravenous infusion and controlled using a motorized syringe press or infusion pump set for 30 minutes. Prior to dosing and as needed thereafter, the area used for dosing was identified by clipping the area free of hair. The injection site(s) was marked and maintained as needed. The dose site was saphenous, left (or right if needed).

Groups: Animals were enrolled into the study groups as listed in Table 12 below.

TABLE 12
Group Assignment and Dose Levels

| | | Dose | Number of |
|---|---|---|---|
| | Dose Level^{b} | Concentration^{b} | Animals |
| Group^{a} | (mg/kg/dose) | (mg/mL) | Males |
| 1 | 10 | 0.44 | 2 |
| 2 | 30 | 1.33 | 2 |
| 3 (Control)^{c} | 0 | 0 | 2 |

^{a}Group 2 initiated dosing at least 3 days following Group 1 dosing initiation.
^{b}Animals were dosed at a volume of 22.5 mL/kg, based on the most recently recorded scheduled body weight.
^{c}Group 3 was dosed with the vehicle control only.

Clinical Observations: Health monitoring was performed on all animals at least twice daily (a.m. and p.m.) and at least once on the day of transfer on or off study. Abnormal findings, if any, were recorded as they were observed for mortality or abnormalities. Food consumption was monitored daily during the pre-dose and dosing phases, except on the day of animal transfer or unless fasted for other study procedures if appropriate. Abnormal findings, if any, were recorded. Cage-side observations were performed once daily during the pre-dose and dosing phase, and abnormal findings, if any, were recorded. Post-dose examinations and cage-side observations were performed on each day of dosing for each dosed animal 4 hours post the start of infusion, and abnormal findings (if any) or an indication of normal was recorded. Detailed observations (including body weight) were performed at least once during the pre-dose phase, on Day-1, and weekly (based on Day-1) throughout the dosing phase, as well as on the last day of the dosing phase. Abnormal findings (if any) or an indication of normal was recorded.

Clinical laboratory procedures: Blood was collected through femoral vein starting twice pre-dose and 24, 48, 96, 168, 240, 336, 504, and 672 hours post-dose, while the post-dose time points were based off the end of the infusion for each animal. The animals were fasted overnight for the pre-dose phase collections and not fasted for the dosing phase. About 2 mL was collected with potassium EDTA for hematology analysis, including red blood cell (erythrocyte) count, hemoglobin, hematocrit, mean corpuscular volume, mean corpuscular hemoglobin, mean corpuscular hemoglobin concentration, red cell distribution width, absolute reticulocyte count, platelet count, white blood cell (leukocyte) count, absolute neutrophil count, absolute lymphocyte count, absolute monocyte count, absolute eosinophil count, absolute basophil count, absolute large unstained cell count, and blood smear. Another 1 mL was collected for clinical chemistry analysis, including glucose, urea nitrogen, creatinine, total protein, albumin, globulin, albumin:globulin ratio, total cholesterol, triglycerides, total bilirubin, aspartate aminotransferase, alanine aminotransferase, alkaline phosphatase, gamma glutamyltransferase, creatine kinase, calcium, inorganic phosphorus, sodium, potassium, and chloride.

Statistics: Means and standard deviations were calculated for the following parameters: absolute body weight; body weight change; and continuous clinical pathology values.

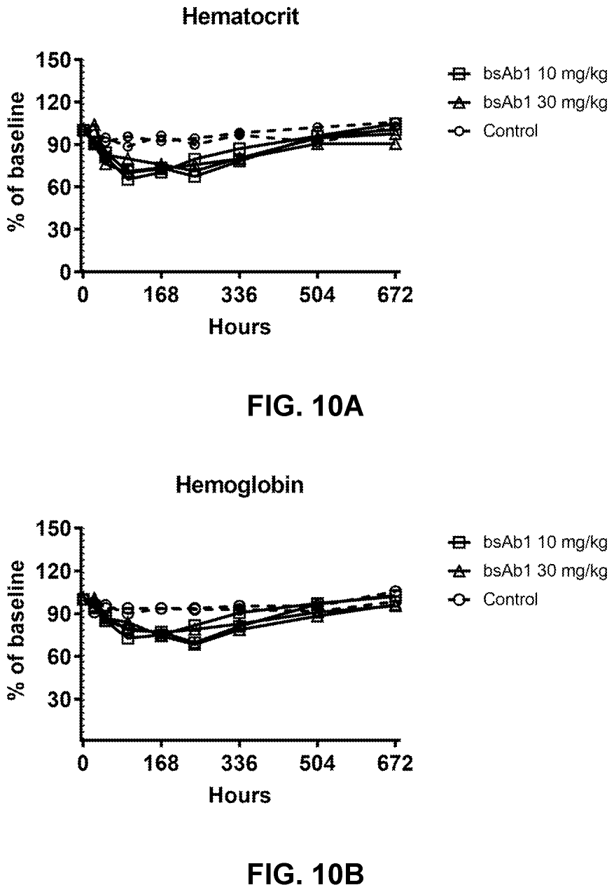

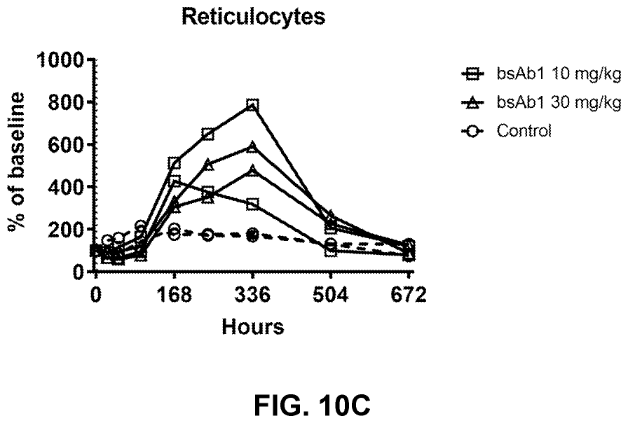

Hematocrit, hemoglobin and reticulocytes data are plotted in FIGS. 10A-10C as percent of baseline (average of two pre-dose measurements) for each individual animal. The results indicate that bsAb1 was tolerated at 10 and 30 mg/kg, and minimal to moderate bsAb1-related anemia and erythroid regenerative response (reticulocytosis) were noted at tolerated doses of ≤30 mg/kg in non-human primates (NHP).

SEQUENCES
Exemplary Multispecific Binding Agents
Sequences of exemplary multispecific antibodies:
(1)
mAb-C x mAb-P in an exemplary format (bsAb1)
mAb-C First polypeptide chain
(VL-variant CH3-Hinge-CH2-CH3knob)
(SEQ ID NO: 48)
DIQMTQSPSSLSASVGDRVTITCRASQSVSSAVAWYQQKPGKAPK
LLIYSASSLYSGVPSRFSGSRSGTDFTLTISSLQPEDFATYYCQQ
SRSRPRTFGQGTKVEIKGQPREPQVCTLPPSRDELTKNQVSLTCL
VRGFYPSDIAVEWESNGQPENNYKTTPPVLDSDGSFFLYSKLTVD
KSRWQQGNVFSCSVMHEALHNHYTQKSLSLSPGKDKTHTCPPCPA
PELLGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALPAPIEKTISKAKGQPREPQVYTLPPSRDELTKNQVSLW
CLVKGFYPSDIAVEWESNGQPENNYKTTPPVLDSDGSFFLYSKLT
VDKSRWQQGNVFSCSVMHEALHNHYTQKSLSLSPGK
mAb-C Second polypeptide chain (VH-variant CH3)
(SEQ ID NO: 49)
EVQLVESGGGLVQPGGSLRLSCAASGFTFSWYYIHWVRQAPGKGL
EWVAYIDSSSGYTEYADSVKGRFTISADTSKNTAYLQMNSLRAED
TAVYYCARGGYLGMDYWGQGTLVTVSSAQPREPQVYTLPPCRDWL
TKNQVSLTCLVKGFYPSDIAVEWESNGQPENNYKTTPPVLDSDGS
FFLYSKLTVDKSRWQQGNVFSCSVMHEALHNHYTQKSLS
LSPGK
mAb-P Third polypeptide chain
(VL-Ck-Hinge-CH2-CH3hole)
(SEQ ID NO: 50)
DIQMTQSPSSLSASVGDRVTITCRASQSVSSAVAWYQQKPGKAPK
LLIYSASSLSSGVPSRFSGSRSGTDFTLTISSLQPEDFATYYCQQ
FGAEPITFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECDKTHTCPPCPA
PELLGGPSVFLFPPKPKDTLMISRTPEVTCVVVDVSHEDPEVKEN
WYVDGVEVHNAKTKPREEQYNSTYRVVSVLTVLHQDWLNGKEYKC
KVSNKALPAPIEKTISKAKGQPREPQVYTLPPSREEMTKNQVSLS
CAVKGFYPSDIAVEWESNGQPENNYKTTPPVLDSDGSFFLVSKLT
VDKSRWQQGNVFSCSVMHEALHNHYTQKSLSLSPGK
mAb-P Fourth polypeptide chain (VH-CH1)
(SEQ ID NO: 51)
EVQLVESGGGLVQPGGSLRLSCAASGFTFRSYYIHWVRQAPGKGL
EWVATISSYGVFTYYADSVKGRFTISADTSKNTAYLQMNSLRAED
TAVYYCARGTTLTPVLDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSC
(2)
mAb-C x mAb-P in an exemplary format
(bsAb1 with silent Fc)
mAb-C First polypeptide chain
(VL-variant CH3-Hinge-CH2-CH3knob) silent Fc
(SEQ ID NO: 52)
DIQMTQSPSSLSASVGDRVTITCRASQSVSSAVAWYQQKPGKAPK
LLIYSASSLYSGVPSRFSGSRSGTDFTLTISSLQPEDFATYYCQQ
SRSRPRTFGQGTKVEIKGQPREPQVCTLPPSRDELTKNQVSLTCL
VRGFYPSDIAVEWESNGQPENNYKTTPPVLDSDGSFFLYSKLTVD
KSRWQQGNVFSCSVMHEALHNHYTQKSLSLSPGKDKTHTCPPCPA
PEAAGGPSVFLFPPKPKDTLMISRTPEVTCVVVDVSHEDPEVKEN
WYVDGVEVHNAKTKPREEQYNSTYRVVSVLTVLHQDWLNGKEYKC
KVSNKALKAPIEKTISKAKGQPREPQVYTLPPSRDELTKNQVSLW
CLVKGFYPSDIAVEWESNGQPENNYKTTPPVLDSDGSFFLYSKLT
VDKSRWQQGNVFSCSVMHEALHNHYTQKSLSLSPGK
mAb-C Second polypeptide chain (VH-variant CH3)
(SEQ ID NO: 49)
EVQLVESGGGLVQPGGSLRLSCAASGFTFSWYYIHWVRQAPGKGL
EWVAYIDSSSGYTEYADSVKGRFTISADTSKNTAYLQMNSLRAED
TAVYYCARGGYLGMDYWGQGTLVTVSSAQPREPQVYTLPPCRDWL
TKNQVSLTCLVKGFYPSDIAVEWESNGQPENNYKTTPPVLDSDGS
FFLYSKLTVDKSRWQQGNVFSCSVMHEALHNHYTQKSLSLSPGK
mAb-P Third polypeptide chain
(VL-Ck-Hinge-CH2-CH3hole) silent Fc
(SEQ ID NO: 53)
DIQMTQSPSSLSASVGDRVTITCRASQSVSSAVAWYQQKPGKAPK
LLIYSASSLSSGVPSRFSGSRSGTDFTLTISSLQPEDFATYYCQQ
FGAEPITFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECDKTHTCPPCPA
PEAAGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALKAPIEKTISKAKGQPREPQVYTLPPSREEMTKNQVSLS
CAVKGFYPSDIAVEWESNGQPENNYKTTPPVLDSDGSFFLVSKLT
VDKSRWQQGNVFSCSVMHEALHNHYTQKSLSLSPGK
mAb-P Fourth polypeptide chain (VH-CH1)
(SEQ ID NO: 51)
EVQLVESGGGLVQPGGSLRLSCAASGFTFRSYYIHWVRQAPGKGL
EWVATISSYGVFTYYADSVKGRFTISADTSKNTAYLQMNSLRAED
TAVYYCARGTTLTPVLDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSC

ADDITIONAL SEQUENCES
mAb-C mAb Heavy Chain (VH)
(SEQ ID NO: 54)
EVQLVESGGGLVQPGGSLRLSCAASGFTFSWYYIHWVRQAPGKGL
EWVAYIDSSSGYTEYADSVKGRFTISADTSKNTAYLQMNSLRAED
TAVYYCARGGYLGMDYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
mAb-C mAb Light Chain (VL)
(SEQ ID NO: 55)
DIQMTQSPSSLSASVGDRVTITCRASQSVSSAVAWYQQKPGKAPK
LLIYSASSLYSGVPSRFSGSRSGTDFTLTISSLQPEDFATYYCQQ
SRSRPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
mAb-P Heavy Chain (VH)
(SEQ ID NO: 56)
EVQLVESGGGLVQPGGSLRLSCAASGFTFRSYYIHWVRQAPGKGL
EWVATISSYGVFTYYADSVKGRFTISADTSKNTAYLQMNSLRAED
TAVYYCARGTTLTPVLDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPGK
mAb-P Light Chain (VL)
(SEQ ID NO: 57)
DIQMTQSPSSLSASVGDRVTITCRASQSVSSAVAWYQQKPGKAPK
LLIYSASSLSSGVPSRFSGSRSGTDFTLTISSLQPEDFATYYCQQ
FGAEPITFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. A multispecific antibody or fragment thereof comprising a first binding domain that binds to CD47 and one of more additional binding domains that bind to one or more targets that are not CD47, wherein the first binding domain comprises: (a) a heavy chain variable (VH) region comprising a VH CDR1, a VH CDR2, and a VH CDR3 amino acid sequence set forth in Table 1; and (b) a light chain variable (VL) region comprising a VL CDR1, a VL CDR2, and a VL CDR3 amino acid sequence set forth in Table 1.

2. The multispecific antibody or fragment thereof of claim 1, wherein the multispecific antibody comprises a second binding domain that binds to PD-L1.

3. The multispecific antibody or fragment thereof of claim 1 or 2, which is a bispecific antibody.

4. A multispecific antibody or fragment thereof with a first binding domain that binds to CD47, wherein the first binding domain comprises:
(a) a heavy chain variable (VH) region comprising:
(1) a VH CDR1 having an amino acid sequence selected from the group consisting of:
(i) SEQ ID NO:1,
(ii) SEQ ID NO:7,
(iii) SEQ ID NO:12,
(iv) SEQ ID NO:13, and
(v) SEQ ID NO: 18;
(2) a VH CDR2 having an amino acid sequence selected from the group consisting of:
(i) SEQ ID NO:2,
(ii) SEQ ID NO:8,
(iii) SEQ ID NO:14,
(iv) SEQ ID NO:19, and
(v) SEQ ID NO:24; and
(3) a VH CDR3 having an amino acid sequence selected from the group consisting of:
(i) SEQ ID NO:3,
(ii) SEQ ID NO:9,
(iii) SEQ ID NO:15, and
(iv) SEQ ID NO:20;
and
(b) a light chain variable (VL) region comprising:
(1) a VL CDR1 having an amino acid sequence selected from the group consisting of:
(i) SEQ ID NO:4,
(ii) SEQ ID NO:10,
(iii) SEQ ID NO:16, and
(iv) SEQ ID NO:21;
(2) a VL CDR2 having an amino acid sequence selected from the group consisting of:
(i) SEQ ID NO:5,
(ii) SEQ ID NO:11, and
(iii) SEQ ID NO:22; and
(3) a VL CDR3 having an amino acid sequence selected from the group consisting of:
(i) SEQ ID NO:6,
(ii) SEQ ID NO:17, and
(iii) SEQ ID NO:23.

5. The multispecific antibody or fragment thereof of claim 4, wherein the multispecific antibody comprises a second binding domain that binds to PD-L1.

6. The multispecific antibody or fragment thereof of claim 4 or 5, which is a bispecific antibody.

7. The multispecific antibody or fragment thereof of claim 5 or 6, wherein the second binding domain comprises:
(a) a heavy chain variable (VH) region comprising:
(1) a VH CDR1 having an amino acid sequence selected from the group consisting of:
(i) SEQ ID NO:27,
(ii) SEQ ID NO:32,
(iii) SEQ ID NO:35,
(iv) SEQ ID NO:36, and
(v) SEQ ID NO:40;
(2) a VH CDR2 having an amino acid sequence selected from the group consisting of:
(i) SEQ ID NO:28,
(ii) SEQ ID NO:33,
(iii) SEQ ID NO:37,
(iv) SEQ ID NO:41, and
(v) SEQ ID NO:45; and
(3) a VH CDR3 having an amino acid sequence selected from the group consisting of:
(i) SEQ ID NO:29,
(ii) SEQ ID NO:34,
(iii) SEQ ID NO:38, and
(iv) SEQ ID NO:42;
and
(b) a light chain variable (VL) region comprising:
(1) a VL CDR1 having an amino acid sequence selected from the group consisting of:
(i) SEQ ID NO:4,
(ii) SEQ ID NO: 10,
(iii) SEQ ID NO:16, and
(iv) SEQ ID NO:21;
(2) a VL CDR2 having an amino acid sequence selected from the group consisting of:
(i) SEQ ID NO:30,
(ii) SEQ ID NO:11, and
(iii) SEQ ID NO:43; and
(3) a VL CDR3 having an amino acid sequence selected from the group consisting of:
(i) SEQ ID NO:31,
(ii) SEQ ID NO:39, and
(iii) SEQ ID NO:44.

8. A multispecific antibody or fragment thereof with a first binding domain that binds to CD47, wherein the first binding domain comprises a heavy chain variable (VH) region comprising:
(1) a VH CDR1 having an amino acid sequence selected from the group consisting of:
(i) SEQ ID NO:1,
(ii) SEQ ID NO:7,
(iii) SEQ ID NO:12,
(iv) SEQ ID NO: 13, and
(v) SEQ ID NO: 18;
(2) a VH CDR2 having an amino acid sequence selected from the group consisting of:
(i) SEQ ID NO:2,
(ii) SEQ ID NO:8,
(iii) SEQ ID NO: 14,
(iv) SEQ ID NO:19, and
(v) SEQ ID NO:24; and
(3) a VH CDR3 having an amino acid sequence selected from the group consisting of:
(i) SEQ ID NO:3,
(ii) SEQ ID NO:9,
(iii) SEQ ID NO: 15, and
(iv) SEQ ID NO:20.

9. The multispecific antibody or fragment thereof of claim 8, wherein the multispecific antibody comprises a second binding domain that binds to PD-L1.

10. The multispecific antibody or fragment thereof of claim 8 or 9, which is a bispecific antibody.

11. The multispecific antibody or fragment thereof of claim 9 or 10, wherein the second binding domain comprises:
(a) a heavy chain variable (VH) region comprising:
(1) a VH CDR1 having an amino acid sequence selected from the group consisting of:
(i) SEQ ID NO:27,
(ii) SEQ ID NO:32,
(iii) SEQ ID NO:35,
(iv) SEQ ID NO:36, and
(v) SEQ ID NO:40;
(2) a VH CDR2 having an amino acid sequence selected from the group consisting of:
(i) SEQ ID NO:28,
(ii) SEQ ID NO:33,
(iii) SEQ ID NO:37,
(iv) SEQ ID NO:41, and
(v) SEQ ID NO:45; and
(3) a VH CDR3 having an amino acid sequence selected from the group consisting of:
(i) SEQ ID NO:29,
(ii) SEQ ID NO:34,
(iii) SEQ ID NO:38, and
(iv) SEQ ID NO:42.

12. A multispecific antibody or fragment thereof with a first binding domain that binds to CD47, wherein the first binding domain comprises a light chain variable (VL) region comprising:
(1) a VL CDR1 having an amino acid sequence selected from the group consisting of:
(i) SEQ ID NO:4,
(ii) SEQ ID NO: 10,
(iii) SEQ ID NO: 16, and
(iv) SEQ ID NO:21;
(2) a VL CDR2 having an amino acid sequence selected from the group consisting of:
(i) SEQ ID NO:5,
(ii) SEQ ID NO:11, and
(iii) SEQ ID NO:22;
(3) a VL CDR3 having an amino acid sequence selected from the group consisting of:
(i) SEQ ID NO:6,
(ii) SEQ ID NO: 17, and
(iii) SEQ ID NO:23.

13. The multispecific antibody or fragment thereof of claim 12, wherein the multispecific antibody comprises a second binding domain that binds to PD-L1.

14. The multispecific antibody or fragment thereof of claim 12 or 13, which is a bispecific antibody.

15. The multispecific antibody or fragment thereof of claim 13 or 14, wherein the second binding domain comprises:
(a) a light chain variable (VL) region comprising:
(1) a VL CDR1 having an amino acid sequence selected from the group consisting of:
(i) SEQ ID NO:4,
(ii) SEQ ID NO:10,
(iii) SEQ ID NO:16, and
(iv) SEQ ID NO:21;
(2) a VL CDR2 having an amino acid sequence selected from the group consisting of:
(i) SEQ ID NO:30,
(ii) SEQ ID NO:11, and
(iii) SEQ ID NO:43; and
(3) a VL CDR3 having an amino acid sequence selected from the group consisting of:
(i) SEQ ID NO:31,
(ii) SEQ ID NO:39, and
(iii) SEQ ID NO:44.

16. A multispecific antibody or fragment thereof with a first binding domain that binds to CD47, wherein the first binding domain comprises all three heavy chain complementarity determining regions (CDRs) or all three light chain CDRs from the antibody designated mAb-C that comprises a VH sequence that is SEQ ID NO:25 and a VL sequence that is SEQ ID NO:26;

17. The multispecific antibody or fragment thereof of claim 16, wherein the multispecific antibody comprises a second binding domain that binds to PD-L1.

18. The multispecific antibody or fragment thereof of claim 16 or 17, which is a bispecific antibody.

19. The multispecific antibody or fragment thereof of claim 17 or 18, wherein the second binding domain comprises all three heavy chain complementarity determining regions (CDRs) or all three light chain CDRs from the antibody designated mAb-P that comprises a VH sequence that is SEQ ID NO:46 and a VL sequence that is SEQ ID NO:47.

20. The multispecific antibody or fragment thereof of claim 16, wherein the first binding domain comprises all three heavy chain CDRs and/or all three light chain CDRs from the antibody designated mAb-C.

21. The multispecific antibody or fragment thereof of claim 20, wherein the multispecific antibody comprises a second binding domain that binds to PD-L1.

22. The multispecific antibody or fragment thereof of claim 20 or 21, which is a bispecific antibody.

23. The multispecific antibody or fragment thereof of claim 21 or 22, wherein the second binding domain comprises all three heavy chain CDRs and all three light chain CDRs from the antibody designated mAb-P.

24. A multispecific antibody or fragment thereof with a first binding domain that binds to CD47, wherein the first binding domain comprises:
(a) a heavy chain variable (VH) region comprising a VH CDR1, a VH CDR2, and a VH CDR3 amino acid sequence set forth in Table 1;
or
(b) a light chain variable (VL) region comprising a VL CDR1, a VL CDR2, and a VL CDR3 amino acid sequence set forth in Table 1.

25. A multispecific antibody or fragment thereof comprising a first binding domain that binds to CD47 and one or more additional binding domains that bind to one or more targets that are not CD47, wherein (a) the multispecific antibody or fragment thereof binds to human CD47 and comprises a heavy chain variable region having an amino acid sequence of SEQ ID NO:25 and a light chain variable region having an amino acid sequence of SEQ ID NO:26 or (b) the multispecific antibody or fragment thereof competes for binding to human CD47 with an antibody comprising a heavy chain variable region having an amino acid sequence of SEQ ID NO:25 and a light chain variable region having an amino acid sequence of SEQ ID NO:26.

26. The multispecific antibody or fragment thereof of claim 24, wherein the multispecific antibody comprises a second binding domain that binds to PD-L1.

27. The multispecific antibody or fragment thereof of any one of claims 24-26, which is a bispecific antibody.

28. The multispecific antibody or fragment thereof of claim 26 or 27, wherein the second binding domain comprises:
(a) a heavy chain variable (VH) region comprising a VH CDR1, a VH CDR2, and a VH CDR3 amino acid sequence set forth in Table 2; and/or
(b) a light chain variable (VL) region comprising a VL CDR1, a VL CDR2, and a VL CDR3 amino acid sequence set forth in Table 2.

29. The multispecific antibody or fragment thereof of claim 24, wherein the first binding domain comprises a heavy chain variable (VH) region comprising a VH CDR1, a VH CDR2, and a VH CDR3 amino acid sequence set forth in Table 1.

30. The multispecific antibody or fragment thereof of claim 29, wherein the multispecific antibody comprises a second binding domain that binds to PD-L1.

31. The multispecific antibody or fragment thereof of claim 29 or 30, which is a bispecific antibody.

32. The multispecific antibody or fragment thereof of claim 30 or 31, wherein the second binding domain comprises a heavy chain variable (VH) region comprising a VH CDR1, a VH CDR2, and a VH CDR3 amino acid sequence set forth in Table 2.

33. The multispecific antibody or fragment thereof of claim 24, wherein the first binding domain comprises a light chain variable (VL) region comprising a VL CDR1, a VL CDR2, and a VL CDR3 amino acid sequence set forth in Table 1.

34. The multispecific antibody or fragment thereof of claim 33, wherein the multispecific antibody comprises a second binding domain that binds to PD-L1.

35. The multispecific antibody or fragment thereof of claim 33 or 34, which is a bispecific antibody.

36. The multispecific antibody or fragment thereof of claim 34 or 35, wherein the second binding domain comprises a light chain variable (VL) region comprising a VL CDR1, a VL CDR2, and a VL CDR3 amino acid sequence set forth in Table 2

37. The multispecific antibody or fragment thereof of claim 24, wherein the first binding domain comprises:
(a) a heavy chain variable (VH) region comprising:
(1) a VH CDR1 having an amino acid sequence selected from the group consisting of SEQ ID NO:1, 7, 12, 13, and 18;
(2) a VH CDR2 having an amino acid sequence selected from the group consisting of SEQ ID NO:2, 8, 14, 19, and 24; and
(3) a VH CDR3 having an amino acid sequence selected from the group consisting of SEQ ID NO:3, 9, 15, and 20;
and
(b) a light chain variable (VL) region comprising:
(1) a VL CDR1 having an amino acid sequence selected from the group consisting of SEQ ID NO:4, 10, 16, and 21;
(2) a VL CDR2 having an amino acid sequence selected from the group consisting of SEQ ID NO:5, 11, and 22; and
(3) a VL CDR3 having an amino acid sequence selected from the group consisting of SEQ ID NO:6, 17, and 23.

38. The multispecific antibody or fragment thereof of claim 37, wherein the multispecific antibody comprises a second binding domain that binds to PD-L1.

39. The multispecific antibody or fragment thereof of claim 37 or 38, which is a bispecific antibody.

40. The multispecific antibody or fragment thereof of claim 37, wherein the first binding domain comprises:
(a) a heavy chain variable (VH) region comprising:
(1) a VH CDR1 having the amino acid sequence of SEQ ID NO: 1;
(2) a VH CDR2 having the amino acid sequence of SEQ ID NO: 2; and
(3) a VH CDR3 having the amino acid sequence of SEQ ID NO: 3;
and
(b) a light chain variable (VL) region comprising:
(1) a VL CDR1 having the amino acid sequence of SEQ ID NO: 4;
(2) a VL CDR2 having the amino acid sequence of SEQ ID NO: 5; and
(3) a VL CDR3 having the amino acid sequence of SEQ ID NO: 6.

41. The multispecific antibody or fragment thereof of claim 40, wherein the multispecific antibody comprises a second binding domain that binds to PD-L1.

42. The multispecific antibody or fragment thereof of claim 40 or 41, which is a bispecific antibody.

43. The multispecific antibody or fragment thereof of claim 41 or 42, wherein the second binding domain comprises:
(a) a heavy chain variable (VH) region comprising:
(1) a VH CDR1 having the amino acid sequence of SEQ ID NO: 27;
(2) a VH CDR2 having the amino acid sequence of SEQ ID NO: 28; and
(3) a VH CDR3 having the amino acid sequence of SEQ ID NO: 29;
and
(b) a light chain variable (VL) region comprising:
(1) a VL CDR1 having the amino acid sequence of SEQ ID NO: 4;
(2) a VL CDR2 having the amino acid sequence of SEQ ID NO: 30; and
(3) a VL CDR3 having the amino acid sequence of SEQ ID NO: 31.

44. The multispecific antibody or fragment thereof of claim 37, wherein the first binding domain comprises:
(a) a heavy chain variable (VH) region comprising:
(1) a VH CDR1 having the amino acid sequence of SEQ ID NO: 7;
(2) a VH CDR2 having the amino acid sequence of SEQ ID NO: 8; and
(3) a VH CDR3 having the amino acid sequence of SEQ ID NO: 9;
and
(b) a light chain variable (VL) region comprising:
(1) a VL CDR1 having the amino acid sequence of SEQ ID NO: 10;
(2) a VL CDR2 having the amino acid sequence of SEQ ID NO: 11; and
(3) a VL CDR3 having the amino acid sequence of SEQ ID NO: 6.

45. The multispecific antibody or fragment thereof of claim 44, wherein the multispecific antibody comprises a second binding domain that binds to PD-L1.

46. The multispecific antibody or fragment thereof of claim 44 or 45, which is a bispecific antibody.

47. The multispecific antibody or fragment thereof of claim 45 or 46, wherein the second binding domain comprises:
(a) a heavy chain variable (VH) region comprising:
(1) a VH CDR1 having the amino acid sequence of SEQ ID NO: 32;
(2) a VH CDR2 having the amino acid sequence of SEQ ID NO: 33; and
(3) a VH CDR3 having the amino acid sequence of SEQ ID NO: 34;
and
(b) a light chain variable (VL) region comprising:
(1) a VL CDR1 having the amino acid sequence of SEQ ID NO: 10;
(2) a VL CDR2 having the amino acid sequence of SEQ ID NO: 11; and
(3) a VL CDR3 having the amino acid sequence of SEQ ID NO: 31.

48. The multispecific antibody or fragment thereof of claim 37, wherein the first binding domain comprises:
(a) a heavy chain variable (VH) region comprising:
(1) a VH CDR1 having the amino acid sequence of SEQ ID NO: 12;
(2) a VH CDR2 having the amino acid sequence of SEQ ID NO: 2; and
(3) a VH CDR3 having the amino acid sequence of SEQ ID NO: 3;
and
(b) a light chain variable (VL) region comprising:
(1) a VL CDR1 having the amino acid sequence of SEQ ID NO: 4;
(2) a VL CDR2 having the amino acid sequence of SEQ ID NO: 5; and
(3) a VL CDR3 having the amino acid sequence of SEQ ID NO: 6.

49. The multispecific antibody or fragment thereof of claim 48, wherein the multispecific antibody comprises a second binding domain that binds to PD-L1.

50. The multispecific antibody or fragment thereof of claim 48 or 49, which is a bispecific antibody.

51. The multispecific antibody or fragment thereof of claim 49 or 50, wherein the second binding domain comprises:
(a) a heavy chain variable (VH) region comprising:
(1) a VH CDR1 having the amino acid sequence of SEQ ID NO: 35;
(2) a VH CDR2 having the amino acid sequence of SEQ ID NO: 28; and
(3) a VH CDR3 having the amino acid sequence of SEQ ID NO: 29;
and
(b) a light chain variable (VL) region comprising:
(1) a VL CDR1 having the amino acid sequence of SEQ ID NO: 4;
(2) a VL CDR2 having the amino acid sequence of SEQ ID NO: 30; and
(3) a VL CDR3 having the amino acid sequence of SEQ ID NO: 31.

52. The multispecific antibody or fragment thereof of claim 37, wherein the first binding domain comprises:
(a) a heavy chain variable (VH) region comprising:
(1) a VH CDR1 having the amino acid sequence of SEQ ID NO: 13;
(2) a VH CDR2 having the amino acid sequence of SEQ ID NO: 14; and
(3) a VH CDR3 having the amino acid sequence of SEQ ID NO: 15;
and
(b) a light chain variable (VL) region comprising:
(1) a VL CDR1 having the amino acid sequence of SEQ ID NO: 16;
(2) a VL CDR2 having the amino acid sequence of SEQ ID NO: 11; and
(3) a VL CDR3 having the amino acid sequence of SEQ ID NO: 17.

53. The multispecific antibody or fragment thereof of claim 52, wherein the multispecific antibody comprises a second binding domain that binds to PD-L1.

54. The multispecific antibody or fragment thereof of claim 52 or 53, which is a bispecific antibody.

55. The multispecific antibody or fragment thereof of claim 53 or 54, wherein the second binding domain comprises:
(a) a heavy chain variable (VH) region comprising:
(1) a VH CDR1 having the amino acid sequence of SEQ ID NO: 36;
(2) a VH CDR2 having the amino acid sequence of SEQ ID NO: 37; and
(3) a VH CDR3 having the amino acid sequence of SEQ ID NO: 38;
and
(b) a light chain variable (VL) region comprising:
(1) a VL CDR1 having the amino acid sequence of SEQ ID NO: 16;
(2) a VL CDR2 having the amino acid sequence of SEQ ID NO: 11; and
(3) a VL CDR3 having the amino acid sequence of SEQ ID NO: 39.

56. The multispecific antibody or fragment thereof of claim 51, wherein the first binding domain comprises:
(a) a heavy chain variable (VH) region comprising:
(1) a VH CDR1 having the amino acid sequence of SEQ ID NO: 18;
(2) a VH CDR2 having the amino acid sequence of SEQ ID NO: 19; and
(3) a VH CDR3 having the amino acid sequence of SEQ ID NO: 20;
and
(b) a light chain variable (VL) region comprising:
(1) a VL CDR1 having the amino acid sequence of SEQ ID NO: 21;
(2) a VL CDR2 having the amino acid sequence of SEQ ID NO: 22; and
(3) a VL CDR3 having the amino acid sequence of SEQ ID NO: 23.

57. The multispecific antibody or fragment thereof of claim 56, wherein the multispecific antibody comprises a second binding domain that binds to PD-L1.

58. The multispecific antibody or fragment thereof of claim 56 or 57, which is a bispecific antibody.

59. The multispecific antibody or fragment thereof of claim 57 or 58, wherein the second binding domain comprises:
(a) a heavy chain variable (VH) region comprising:
(1) a VH CDR1 having the amino acid sequence of SEQ ID NO: 40;
(2) a VH CDR2 having the amino acid sequence of SEQ ID NO: 41; and
(3) a VH CDR3 having the amino acid sequence of SEQ ID NO: 42;
and
(b) a light chain variable (VL) region comprising:
(1) a VL CDR1 having the amino acid sequence of SEQ ID NO: 21;
(2) a VL CDR2 having the amino acid sequence of SEQ ID NO: 43; and
(3) a VL CDR3 having the amino acid sequence of SEQ ID NO: 44.

60. The multispecific antibody or fragment thereof of claim 37, wherein the first binding domain comprises:
(a) a heavy chain variable (VH) region comprising:
(1) a VH CDR1 having the amino acid sequence of SEQ ID NO: 1;
(2) a VH CDR2 having the amino acid sequence of SEQ ID NO: 24; and
(3) a VH CDR3 having the amino acid sequence of SEQ ID NO: 3;
and
(b) a light chain variable (VL) region comprising:
(1) a VL CDR1 having the amino acid sequence of SEQ ID NO: 4;
(2) a VL CDR2 having the amino acid sequence of SEQ ID NO: 5; and
(3) a VL CDR3 having the amino acid sequence of SEQ ID NO: 6.

61. The multispecific antibody or fragment thereof of claim 60, wherein the multispecific antibody comprises a second binding domain that binds to PD-L1.

62. The multispecific antibody or fragment thereof of claim 60 or 61, which is a bispecific antibody.

63. The multispecific antibody or fragment thereof of claim 61 or 62, wherein the second binding domain comprises:
(a) a heavy chain variable (VH) region comprising:
(1) a VH CDR1 having the amino acid sequence of SEQ ID NO: 27;
(2) a VH CDR2 having the amino acid sequence of SEQ ID NO: 45; and
(3) a VH CDR3 having the amino acid sequence of SEQ ID NO: 29;
and
(b) a light chain variable (VL) region comprising:
(1) a VL CDR1 having the amino acid sequence of SEQ ID NO: 4;
(2) a VL CDR2 having the amino acid sequence of SEQ ID NO: 30; and
(3) a VL CDR3 having the amino acid sequence of SEQ ID NO: 31.

64. The multispecific antibody or fragment thereof of any one of claims 24-63, wherein the VH region or VL region further comprises human framework sequences.

65. The multispecific antibody or fragment thereof of claim 64, wherein the VH region and VL region further comprises human framework sequences.

66. The multispecific antibody or fragment thereof of any one of claims 24-63, wherein the VH region or VL region further comprises a framework 1 (FR1). a framework 2 (FR2), a framework 3 (FR3) and/or a framework 4 (FR4) sequence.

67. The multispecific antibody or fragment thereof of claim 66, wherein the VH region and VL region further comprises a framework 1 (FR1). a framework 2 (FR2), a framework 3 (FR3) and/or a framework 4 (FR4) sequence.

68. The multispecific antibody or fragment of any one of claims 1-67, wherein the antibody is a recombinant antibody.

69. The multispecific antibody or fragment thereof of any one of claims 1-68 which is a Fab, Fab′, F(ab′)₂, Fv, scFv, (scFv) 2, single chain antibody molecule, dual variable region antibody, single variable region antibody, linear antibody, V region, or a multispecific antibody formed from antibody fragments.

70. A multispecific antibody comprising: (a) a first polypeptide chain having an amino acid sequence of SEQ ID NO:48, a second polypeptide chain having an amino acid sequence of SEQ ID NO:49, a third polypeptide chain having an amino acid sequence of SEQ ID NO:50, and a fourth polypeptide chain having an amino acid sequence of SEQ ID NO:51, or (b) a first polypeptide chain having an amino acid sequence of SEQ ID NO:52, a second polypeptide chain having an amino acid sequence of SEQ ID NO:49, a third polypeptide chain having an amino acid sequence of SEQ ID NO:53, and a fourth polypeptide chain having an amino acid sequence of SEQ ID NO:51.

71. The multispecific antibody or fragment thereof of any one of claims 1-70 which is conjugated or recombinantly fused to a diagnostic agent, detectable agent or therapeutic agent.

72. The multispecific antibody or fragment thereof of claim 71, wherein the therapeutic agent is a chemotherapeutic agent, cytotoxin, or drug.

73. A binding agent that binds to essentially the same epitope of human CD47 as a multispecific antibody or fragment thereof of any one of claims 1-72.

74. The binding agent of claim 73, which is a multispecific antibody or fragment thereof.

75. The binding agent of claim 73, which comprises a non-antibody protein scaffold.

76. The binding agent of claim 75, wherein the non-antibody protein scaffold comprises a fibronectin scaffold, an anticalin, an adnectin, an affibody, a DARPin, a fynomer, an affitin, an affilin, an avimer, a cysteine-rich knottin peptide, or an engineered Kunitz-type inhibitor.

77. A binding agent that competes for the binding to human CD47 as a multispecific antibody or fragment thereof of any one of claims 1-72.

78. The binding agent of claim 77, wherein the binding agent is an antibody or fragment thereof.

79. One or more vectors comprising one or more polynucleotides encoding the multispecific antibody or fragment thereof of any one of claims 1-72.

80. A pharmaceutical composition that comprises the multispecific antibody or fragment thereof of any one of claims 1-72, and a pharmaceutically acceptable carrier.

81. A method for treating a cancer or a tumor in a subject comprising administering to the subject the multispecific antibody or fragment thereof of any one of claims 1-72 or the pharmaceutical composition of claim 80.

82. A method for alleviating one or more symptoms associated with a cancer or a tumor in a subject comprising administering to the subject the multispecific antibody or fragment thereof of any one of claims 1-72 or the pharmaceutical composition of claim 80.

83. A method for decreasing tumor size in a subject with a tumor comprising administering to the subject the multispecific antibody or fragment thereof of any one of claims 1-72 or the pharmaceutical composition of claim 80.

84. A method for enhancing tumor cell removal in a subject with a tumor comprising administering to the subject the multispecific antibody or fragment thereof of any one of claims 1-72 or the pharmaceutical composition of claim 80.

85. A method for treating a phagocytic cell dysfunctional disease, disorder or condition in a subject comprising administering to the subject the multispecific antibody or fragment thereof of any one of claims 1-72 or the pharmaceutical composition of claim 80.

86. A method for increasing immune cell phagocytosis in a subject comprising administering to the subject the multispecific antibody or fragment thereof of any one of claims 1-72 or the pharmaceutical composition of claim 80.

87. The method of claim 86, wherein the immune cell is a macrophage, a neutrophil, a dendritic cell, or a B lymphocyte.

88. The method of claim 86 or 87, wherein the subject is diagnosed with a cancer or a tumor.

89. A method for treating a T cell dysfunctional disease, disorder or condition in a subject comprising administering to the subject the multispecific antibody or fragment thereof of any one of claims 1-72 or the pharmaceutical composition of claim 80.

90. The method of claim 89, wherein the T cell dysfunctional disease, disorder or condition is tumor immunity.

91. A method for enhancing T cell function in a subject comprising administering to the subject the multispecific antibody or fragment thereof of any one of claims 1-72 or the pharmaceutical composition of claim 80.

92. The method of claim 91, wherein the T cell function is secretion of cytokines.

93. The method of claim 91, wherein the T cell function is removal of tumor cells.

94. The method of any one of claims 91-93, wherein the subject is diagnosed with a cancer or a tumor.

95. The method of any one of claims 81-94, wherein the subject is administered one or more therapeutic agents in combination with the multispecific antibody or fragment thereof or the pharmaceutical composition.
